(19) United States
(12) Patent Application Publication
(10) Pub. No.: US 2021/0085684 A1
(43) Pub. Date: Mar. 25, 2021

(54) PDE9 INHIBITORS FOR TREATMENT OF PERIPHERAL DISEASES

(71) Applicant: Imara Inc., Boston, MA (US)

(72) Inventors: Niels Svenstrup, Kobenhavn V (DK); Anna I. Parachikova, Copenhagen K (DK); James McArthur, Arlington, MA (US)

(21) Appl. No.: 17/067,588

(22) Filed: Oct. 9, 2020

Related U.S. Application Data
(63) Continuation of application No. 16/315,365, filed on Jan. 4, 2019.
(60) Provisional application No. 62/448,414, filed on Jan. 20, 2017.
(60) Provisional application No. 62/359,080, filed on Jul. 6, 2016.

(51) Int. Cl. A61K 31/519; A61P 7/06; A61K 31/542

(52) U.S. Cl. CPC A61K 31/519; A61K 31/17; A61K 31/542; A61P 7/06

(57) ABSTRACT

The present invention relates to PDE9 inhibitors, their synthesis, and their use for treatment of benign prostate hyperplasia, beta thalassemia, and sickle cell disease.

### REFERENCE TO RELATED APPLICATIONS

The present application is a continuation of U.S. patent application Ser. No. 16/315,365, filed Jan. 4, 2019, which is a U.S. National Phase of International Application No. PCT/US2017/040160, which claims priority to U.S. Provisional Application No. 62/359,080, filed Jul. 6, 2016, and U.S. Provisional Application No. 62/448,414, filed Jan. 20, 2017, the contents of each of which are incorporated herein by reference in their entirety.

### FIELD OF THE INVENTION

The present invention relates to cyclic guanylate monophosphate (cGMP)-specific phosphodiesterase type 9 inhibitors (hereinafter referred to as PDE9 inhibitors).

### BACKGROUND OF THE INVENTION

Phosphodiesterases (PDEs) are a family of enzymes degrading cyclic nucleotides and thereby regulating the cellular levels of second messengers throughout the entire body. PDEs represent attractive drug targets, as proven by a number of compounds that have been introduced to clinical testing and the market, respectively. PDEs are encoded by 21 genes that are functionally separated into 11 families differing with respect to kinetic properties, substrate selectivity, expression, localization pattern, activation, regulation factors and inhibitor sensitivity. The function of PDEs is the degradation of the cyclic nucleotide monophosphates cyclic Adenosine Monophosphate (cAMP) and/or Guanosine Monophosphate (cGMP), which are important intracellular mediators involved in numerous vital processes including the control of neurotransmission and smooth muscle contraction and relaxation.

PDE9 is cGMP specific (Km cAMP is >1000× for cGMP) and is hypothesized to be a key player in regulating cGMP levels as it has the lowest Km among the PDEs for this nucleotide. PDE9 is expressed throughout the brain at low levels with the potential for regulating basal cGMP.

In the periphery, PDE9 expression is highest in prostate, intestine, kidney and haematopoietic cells, enabling therapeutic potential in various non-CNS indications.

Benign prostate hyperplasia (BPH) is one of the most prevalent conditions in the aging male population and represents a major health problem (Ueckert S et al., Expert Rev Clin Pharmacol. 2013 May; 6(3):323-32). BPH results in the formation of large nodules in the periurethral region of the prostate, which could lead to urinary tract obstruction. BPH is predominantly the result of a stromal proliferative process, and a significant component of prostatic enlargement results from smooth-muscle proliferation. The current pharmacological treatment of BPH includes α1 adrenergic blockers, 5-α-reductase inhibitors and more recently the PDE5 inhibitor tadalafil. PDE5 inhibitors are known to mediate smooth muscle relaxation via increased cGMP levels. The cGMP specific PDE9 is expressed at high levels in the prostate and PDE9 inhibition may thus offer potential antiproliferative benefits for BPH.

PDE9 is widely distributed in the urothelial epithelium of human lower urinary tract and PDE9 inhibition may be beneficial in lower urinary tract dysfunctional epithelium (LUDE) disease (Nagasaki et al., BJU Int. 2012 March; 109(6):934-40). Dysfunctional lower urinary tract epithelium can affect the bladder, urethra, labia or vaginal introitus in women, and the prostatic ducts and urethra in men (Parsons L C et al., 2002).

PDE9 expression has been shown in murine corpus cavernosum and chronic PDE9 inhibition was demonstrated to result in amplified NO-cGMP mediated cavernosal responses and thereby opening for potential benefit in erectile dysfunction (DaSilva et al., Int J Impot Res. 2013 March-April; 25(2):69-73). Currently approved treatment for erectile dysfunction is the class of PDE5 inhibitors, increasing cGMP in the smooth muscle cells lining the blood vessels supplying the corpus cavernosum of the penis.

cGMP PDE inhibition has been shown to enhance muscle microvascular blood flow and glucose uptake response to insulin (Genders et al., Am J Physiol Endocrinol Metab. 2011 August; 301(2):E342-50). The targeting of cGMP specific PDE9, which is expressed in muscle and blood vessels may provide a promising avenue for enhancing muscle insulin sensitivity and thereby be beneficial for the treatment of type 2 diabetes.

PDE9 inhibition may represent a novel and first line treatment for Sickle Cell Disease (SCD), a genetic disorder leading to vaso-occlusive processes responsible for much of the mortality in SCD patients. SCD disease results from a point mutation in the hemoglobin (HBB) gene producing abnormal sickle hemoglobin (HbS), which polymerizes and creates rigid and sticky sickled red blood cells. Sickled red blood cells result in chronic inflammation, elevated cell adhesion, oxidative stress, and endothelial dysfunction culminating in vaso-occlusive processes.

There is to date no cure for SCD. Treatment options include blood transfusion and treatment with the anti-cancer agent hydroxyurea. Blood transfusions correct anemia by increasing the number of normal, non-sickled red blood cells in circulation. Regular transfusion therapy can help prevent recurring strokes in children at high risk. Hydroxyurea (HU) has been approved for the treatment of SCD and shown to reduce the frequency of painful crisis and hospitalization. The mechanism by which HU is hypothesized to ameliorate the symptoms of SCD is two-fold; a) increase in non-sickled fetal haemoglobin production and b) decrease in cell adhesion. Specifically, HU a) increases fetal non-sickled haemoglobin production via cGMP signalling, which has been shown to result in increased red blood cell survival and b) increases nitric oxide and cGMP levels, thereby decreasing adhesion and increasing survival. In summary, the evidence to date supports the notion that both mechanisms by which hydroxyurea promotes benefits in SCD are mediated via increased cGMP.

Unfortunately, HU is often poorly tolerated and its widespread use is limited by concerns about its potential impact on fertility and reproduction; challenges achieving and maintaining an efficacious dose due to its hematologic toxicities; and requirements for monthly monitoring (Heeney et al., Pediatr Cin North Am., 55(2):483-501 (2008)). In fact, it is estimated that only 1 out of 4 adult patients, and possibly even fewer, are treated with this drug (Stettler et al., JAMA., 313:1671-2 (2015)). In addition, many patients are dosed with sub-efficacious doses of HU due to these challenges. Thus, novel, safe, and effective treatments that can be safely employed globally to prevent the morbid complications of SCD in patients of all ages are urgently needed.

Further, PDE9 inhibitors may be used to treat thalassemia disorders, such as beta-thalassemia, a group of genetic blood disorders resulting in the synthesis of little or no hemoglobin beta chains. Symptoms of beta thalassemia include anemia, a lack of oxygen in many parts of the body, pulmonary hypertension, thrombotic events, infection, endocrine dysfunction and leg ulcers. Conventional therapies include regular transfusions of red blood cells. However, repeated transfusions cause iron overload and many side effects (de Dreuzy et al., Biomed J., vol. 39(1):24-38 (2016)). New therapies are highly needed.

WO 2012/040230 discloses PDE9 inhibitors with imidazotriazinone backbone for the use as a medicament in the treatment of PDE9 associated diseases, including CNS and neurodegenerative disorders.

WO 2008/139293 and WO 2010/084438 both disclose amino-heterocyclic compounds that are PDE9 inhibitors and their use in treating neurodegenerative and cognitive disorders.

### SUMMARY OF THE INVENTION

There is a constant need for improved treatment of the peripheral diseases benign prostate hyperplasia (BPH), urinary tract dysfunctional epithelium disease, erectile dysfunction, type 2 diabetes, beta thalassemia, and sickle cell disease (SCD) and for that purpose the use of PDE9 inhibitors may be very useful. Since PDE9 is expressed throughout the brain at with the potential basal cGMP and thus signalling cascades shown to regulate synaptic transmission, it is important that PDE9 inhibitors for the treatment of peripheral diseases have a low blood brain barrier penetration (BBB penetration) to avoid potential centrally-mediated side effects.

The present invention provides novel PDE9 inhibitors that have been shown to have a low blood brain barrier penetration and thus may be particularly useful for the treatment of peripheral diseases such as benign prostate hyperplasia (BPH), urinary tract dysfunctional epithelium disease, erectile dysfunction, type 2 diabetes and sickle cell disease (SCD). Further, the PDE9 inhibitors of the present invention are significantly stronger PDE9 inhibitors than PDE1 inhibitors. This PDE inhibition selectivity is important as PDE1 is expressed in heart and testes and inhibition of these PDE1 isoforms is thought to be a potential cause of cardiovascular and reproductive side effects.

The following compounds are encompassed by the invention:

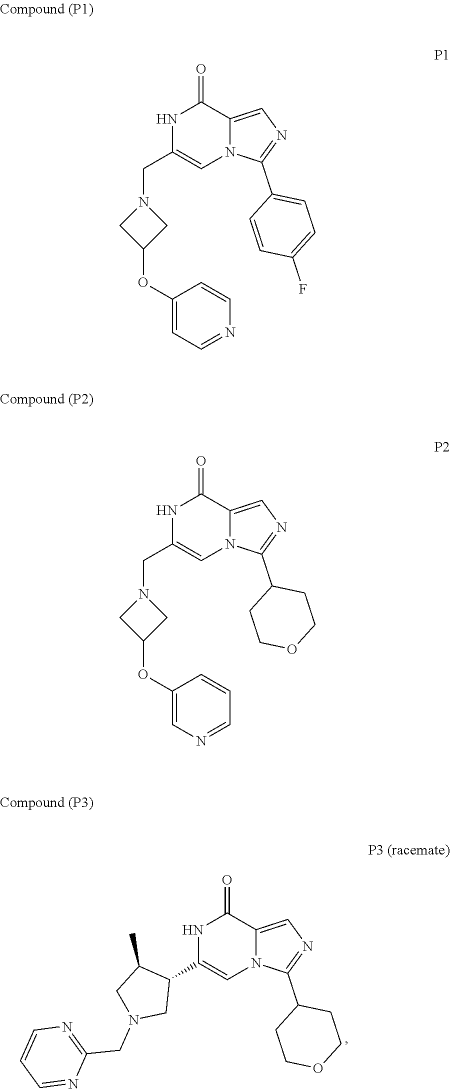 [SMILES: O=c1[nH]c(CN2CC(Oc3ccncc3)C2)cn2c(-c3ccc(F)cc3)ncc12 ; O=c1[nH]c(CN2CC(Oc3cccnc3)C2)cn2c(C3CCOCC3)ncc12 ; C[C@@H]1CN(Cc2ncccn2)C[C@H]1c1cn2c(C3CCOCC3)ncc2c(=O)[nH]1]

racemate and enantiomerically pure variants of compound P3.

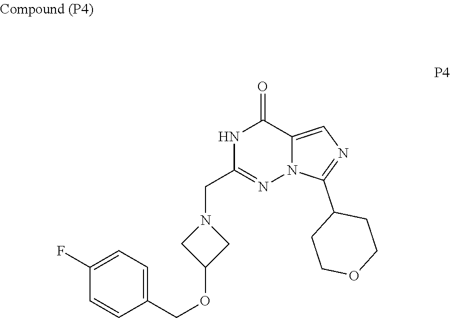 [SMILES: O=c1[nH]c(CN2CC(OCc3ccc(F)cc3)C2)nn2c(C3CCOCC3)ncc12]

Another aspect of the invention is directed to synthesis of P1, P2, P3 and P4. A still further aspect of the invention is directed to the enantioselective synthesis of compound P3 comprising the conversion of the intermediate compound rac-35 to (S,S)-35.

A further aspect of the invention includes methods of using PDE9 inhibitors of the present invention, e.g., to treat beta thalassemia and/or sickle cell disease.

### BRIEF DESCRIPTION OF THE DRAWINGS

FIG. 1 is a graph demonstrating that PDE9 inhibitors of the present invention and hydroxyurea (HU) act through different mechanisms. Abbreviations: cGMP=cyclic guanosine monophosphate; GMP=guanosine monophosphate; GTP=guanosine-5′-triphosphate; HbF=fetal hemoglobin; NO=nitric oxide; PKG=protein kinase G; PDE9=phosphodiesterase 9; RBC=red blood cell; WBC=white blood cell. It is revised from Almeida et al., Blood, vol. 120(14):2879 (2012).

FIG. 2 is a graph showing the effect of Compound P3.1 vs. hydroxyurea on cGMP concentrations in K562 cells. Abbreviations: cGMP=cyclic guanosine monophosphate; SD=standard deviation.

FIG. 3 is a graph showing the effect of Compound P3.1 vs. hydroxyurea on percentage of HbF positive K562 cells. Abbreviations: HbF=fetal hemoglobin; SD=standard deviation.

FIG. 4 is a graph showing the effect of Compound P3.1 vs. hydroxyurea on HbF production in CD34+ derived red blood cells from SCD subjects. Abbreviations: HbF=fetal hemoglobin; MFI=mean fluorescence intensity.

FIG. 5A is a graph showing the effect of Compound P3.1 vs. hydroxyurea on percentage of HbF positive and sickled red blood cells in Berkeley Sickle Cell Transgenic Mice. Abbreviations: HbF=fetal hemoglobin; RBC=red blood cell; SD=standard deviation.

FIG. 5B is a graph showing the effect of Compound P3.1 and hydroxyurea on neutrophil levels in Berkeley Sickle Cell Transgenic Mice.

FIG. 5C is a graph showing spleen weights of the Berkeley Sickle Cell Transgenic Mice treated by vehicle, Compound P3.1, or HU.

FIG. 5D is a graph showing bilirubin levels of the Berkeley Sickle Cell Transgenic Mice treated by vehicle, Compound P3.1, or HU.

FIG. 6 is a graph showing the effect of Compound P3.1 vs. hydroxyurea vs. Compound P3.1 in combination with hydroxyurea on microvascular stasis in HbSS-Townes Mice. Abbreviations: SD=standard deviation; % Stasis=the number of static (no flow) venules counted 1 and 4 hours after re-oxygenation divided by the number of flowing venules selected for analysis prior to hypoxia times 100.

FIG. 7A is a graph showing the effect of Compound P3.1 vs. hydroxyurea vs. Compound P3.1 in combination with hydroxyurea on percentage of HbF positive and sickled red blood cells in HbSS-Townes Mice. Abbreviations: HbF=fetal hemoglobin; RBC=red blood cell; SD=standard deviation.

FIG. 7B is a graph showing % occluded blood vessels in HbSS-Townes Mice after treatments of Compound P3.1, HU, or a combination of Compound P3.1 and HU.

FIG. 8A and FIG. 8B are graphs showing the concentrations of Compound P3.1 vs. AF27873 in brain and eyes of C57Bl/6J Mice. Abbreviations: Conc.=concentration.

FIG. 9A and FIG. 9B are results from a micro-channel assay showing Compound P3.1 reduces neutrophil adhesion to TNF-α activated human endothelial cells.

FIGS. 10A, 10B, and 10C are results from another micro-channel assay showing Compound P3.1 reduces neutrophil and RBC adhesions to TNF-α activated human endothelial cells.

### DETAILED DESCRIPTION OF THE INVENTION

### I. Compounds of the Invention

One aspect of the present invention provides a PDE9-inhibiting compound or a PDE9 inhibitor that may be used to treat sickle cell disease (SCD). The PDE9 inhibitors of the present invention have been shown to have a low blood brain barrier penetration and thus may be particularly useful for the treatment of peripheral diseases such as benign prostate hyperplasia (BPH), urinary tract dysfunctional epithelium disease, erectile dysfunction, type 2 diabetes and sickle cell disease (SCD). Further, the PDE9 inhibitors of the present invention are significantly stronger PDE9 inhibitors than PDE1 inhibitors. This PDE inhibition selectivity is important as PDE1 is expressed in heart and testes and inhibition of these PDE1 isoforms is thought to be a potential cause of cardiovascular and reproductive side effects.

### PDE9 Inhibitors

In the context of the present invention a compound is considered to be a PDE9 inhibitor if the amount required to reach the IC₅₀ level of any of the three PDE9 isoforms is 10 micromolar or less, preferably less than 9 micromolar, such as 8 micromolar or less, such as 7 micromolar or less, such as 6 micromolar or less, such as 5 micromolar or less, such as 4 micromolar or less, such as 3 micromolar or less, more preferably 2 micromolar or less, such as 1 micromolar or less, in particular 500 nM or less. In preferred embodiments the required amount of PDE9 inhibitor required to reach the IC₅₀ level of PDE9 is 400 nM or less, such as 300 nM or less, 200 nM or less, 100 nM or less, or even 80 nM or less, such as 50 nM or less, for example 25 nM or less.

Throughout this application the notations IC₅₀ and IC50 are used interchangeably.

In some embodiments, the PDE9 inhibitor of the present invention has low or no blood brain barrier penetration. For example, the ratio of the concentration of a PDE9 inhibitor of the present invention in the brain to the concentration of it in the plasma (brain/plasma ratio) may be less than about 0.50, about 0.40, about 0.30, about 0.20, about 0.10, about 0.05, about 0.04, about 0.03, about 0.02, or about 0.01. The brain/plasma ratio may be measured 30 min or 120 min after administration of the PDE9 inhibitor.

### Isomeric Forms

Where compounds of the present invention contain one or more chiral centers reference to any of the compounds will, unless otherwise specified, cover the enantiomerically or diastereomerically pure compound as well as mixtures of the enantiomers or diastereomers in any ratio.

In one embodiment, the PDE9 inhibiting compounds of the present invention that are used to treat sickle cell disease comprise an imidazopyrazinone backbone. They may have structure (I) (also referred to as compounds of formula (I))

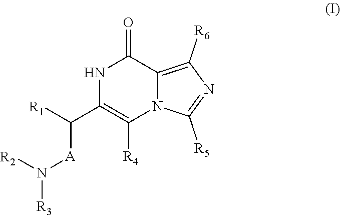

wherein R2 is cyclized with either R1 or R3,
wherein R1, R2 and R3 are
R1, when cyclized with R2, is

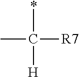

wherein R7 is selected from the group consisting of H, —CH₃, —C₂H₅, and —C₃H₇,
wherein * denotes the cyclization point, and
R1, when not cyclized, is selected from the group consisting of
   H and

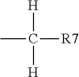

wherein R7 is selected from the group consisting of H, —CH₃, —C₂H₅, and —C₃H₇
R2 is a compound selected from the group consisting of

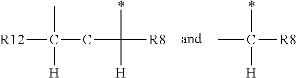

wherein R8 and R12 independently are selected from the group consisting of H, —CH₃, —C₂H₅, and —C₃H₇
   wherein * denotes the cyclization point, and
R3, when cyclized with R2, is

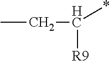

wherein * denotes the cyclization point, and
wherein R9 is selected from the group consisting of H, C₁-C₆ alkyl, substituted C₁-C₆ alkyl, branched C₃-C₆ alkyl, C₃-C₆ cycloalkyl, substituted C₃-C₆ cycloalkyl, C₆-C₁₀ aryl, substituted C₆-C₁₀ aryl, C₃-C₉ heteroaryl, substituted C₃-C₉ heteroaryl, C₁-C₆ alkoxy, substituted C₁-C₆ alkoxy, branched C₃-C₆ alkoxy, C₃-C₆ cycloalkoxy, substituted C₃-C₆ cycloalkoxy, C₆-C₁₀ aryloxy, substituted C₆-C₁₀ aryloxy, C₃-C₉ heteroaryloxy, substituted C₃-C₉ heteroaryloxy; and
R3, when not cyclized, is

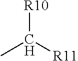

wherein
   R10 is selected from the group consisting of H, —CH₃, and —C₂H₅; and
   R11 is selected from the group consisting of C₆-C₁₀ aryl, substituted C₆-C₁₀ aryl, C₃-C₉ heteroaryl, substituted C₃-C₉ heteroaryl;
R4 is selected from the group consisting of hydrogen, —CH₃, —C₂H₅, —C₃H₇, —CF₃, —CN, F and Cl;
R5 is selected from the group consisting of C₆-C₁₀ aryl, substituted C₆-C₁₀ aryl, C₃-C₉ heteroaryl, substituted C₃-C₉ heteroaryl, C₃-C₆ heterocyclyl, substituted C₃-C₆ heterocyclyl, C₃-C₆ cycloalkyl, and substituted C₃-C₆ cycloalkyl;
R6 is selected from the group consisting of hydrogen, F, Cl, CN, —CH₃, —C₂H₅, —C₃H₇, and —CF₃;
A is absent or —CH₂—;
   and tautomers and pharmaceutically acceptable acid addition salts thereof, and polymorphic forms thereof.

Non-limiting examples of PDE9-inhibiting compounds of formula (I) are disclosed in WO 2013/053690, the contents of which are incorporated herein by reference in their entirety.

For example, the PDE9 inhibitor with an imidazopyrazinone backbone may be selected from the group consisting of:

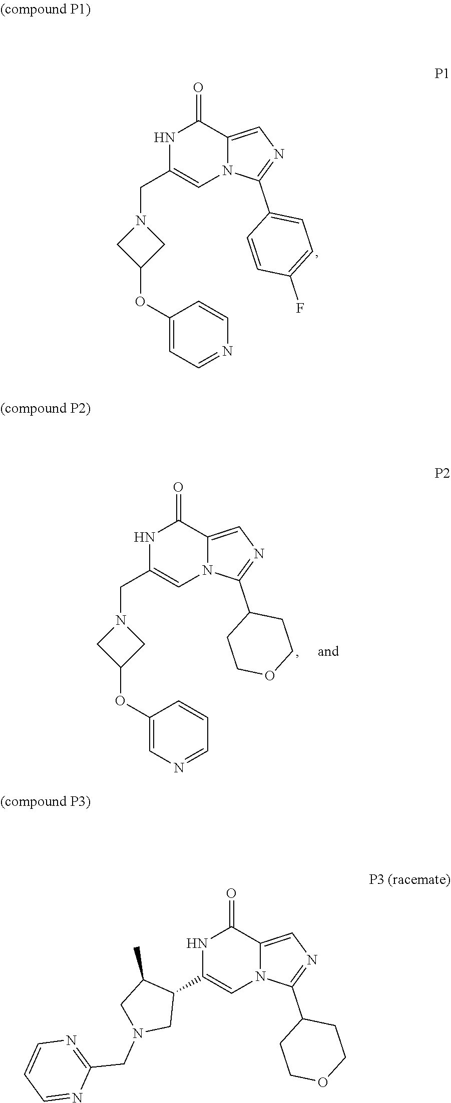 [SMILES: O=c1[nH]c(CN2CC(Oc3ccncc3)C2)cn2c(-c3ccc(F)cc3)ncc12 ; O=c1[nH]c(CN2CC(Oc3cccnc3)C2)cn2c(C3CCOCC3)ncc12 ; C[C@@H]1CN(Cc2ncccn2)C[C@H]1c1cn2c(C3CCOCC3)ncc2c(=O)[nH]1]

in racemic form and in enantiomerically enriched or pure form.

In another embodiment, the PDE9 inhibiting compounds of the present invention that are used to treat sickle cell disease comprise an imidazotriazinone backbone. They may have structure (II) (also referred to as compounds of formula (II))

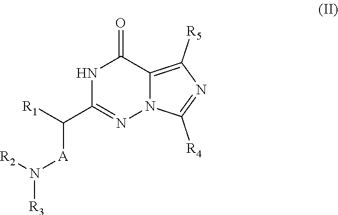

wherein R2 is cyclized with either R1 or R3,
wherein R1, R2 and R3 are
R1, when cyclized with R2, is

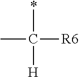

wherein R6 is selected from the group consisting of H, —CH₃, —C₂H₅, and —C₃H₇,
wherein * denotes the cyclization point, and
R1, when not cyclized, is selected from the group consisting of
   H and

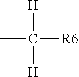

wherein R6 is selected from the group consisting of H, —CH₃, —C₂H₅, and —C₃H₇
R2 is a compound selected from the group consisting of

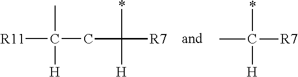

wherein R7 and R11 independently are selected from the group consisting of H, —CH₃, —C₂H₅, and —C₃H₇
   wherein * denotes the cyclization point, and
R3, when cyclized with R2, is

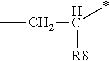

wherein * denotes the cyclization point, and
wherein R8 is selected from the group consisting of H, C₁-C₆ alkyl, substituted C₁-C₆ alkyl, branched C₃-C₆ alkyl, C₃-C₆ cycloalkyl, substituted C₃-C₆ cycloalkyl, C₆-C₁₀ aryl, substituted C₆-C₁₀ aryl, C₃-C₉ heteroaryl, substituted C₃-C₉ heteroaryl, C₁-C₆ alkoxy, substituted C₁-C₆ alkoxy, branched C₃-C₆ alkoxy, C₃-C₆ cycloalkoxy, substituted C₃-C₆ cycloalkoxy, C₆-C₁₀ aryloxy, substituted C₆-C₁₀ aryloxy, C₃-C₉ heteroaryloxy, substituted C₃-C₉ heteroaryloxy; and
R3, when not cyclized, is

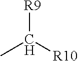

wherein
   R9 is selected from the group consisting of H, —CH₃, and —C₂H₅; and
   R10 is selected from the group consisting of C₆-C₁₀ aryl, substituted C₆-C₁₀ aryl, C₃-C₉ heteroaryl, substituted C₃-C₉ heteroaryl
R4 is selected from the group consisting of C₆-C₁₀ aryl, substituted C₆-C₁₀ aryl, C₃-C₉ heteroaryl, substituted C₃-C₉ heteroaryl, C₃-C₆ heterocyclyl, substituted C₃-C₆ heterocyclyl, C₃-C₆ cycloalkyl, and substituted C₃-C₆ cycloalkyl;
R5 is selected from the group consisting of hydrogen, F, Cl, CN, —CH₃, —C₂H₅, —C₃H₇, and —CF₃;
A is absent or —CH₂—,
   and tautomers and pharmaceutically acceptable acid addition salts thereof, and polymorphic forms thereof.

Non-limiting examples of PDE9 inhibitors of formula (II) are disclosed in WO 2013/110768, the contents of which are incorporated herein by reference in their entirety.

For example, the PDE9 inhibitor with an imidazotriazinone backbone may be

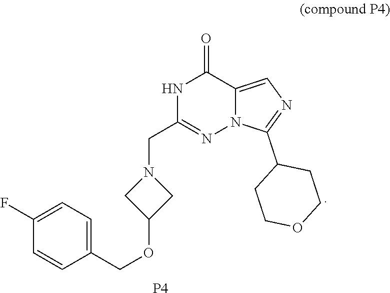

### Non-limiting Embodiments of the Invention

The following notation is applied: an embodiment of the invention is identified as Ei, where i is an integer indicating the number of the embodiment. An embodiment Ei′ specifying a specific embodiment a previously listed embodiment Ei is identified as Ei′(Ei), e.g. E2(E1) means “in an embodiment E2 of embodiment E1”.

Where an embodiment is a combination of two embodiments the notation is similarly Ei″(Ei and Ei′), e.g. E3(E2 and E1) means “in an embodiment E3 of any of embodiments E2 and E1”

Where an embodiment is a combination of more than two embodiments the notation is similarly Ei′″(Ei, Ei′ and Ei″), e.g. E4(E1, E2 and E3) means “in an embodiment E4 of any of embodiments E1, E2 and E3”

Embodiments of the present invention include but not limited to the following embodiments.

In a first embodiment E1 the present invention relates to compounds having the following structure

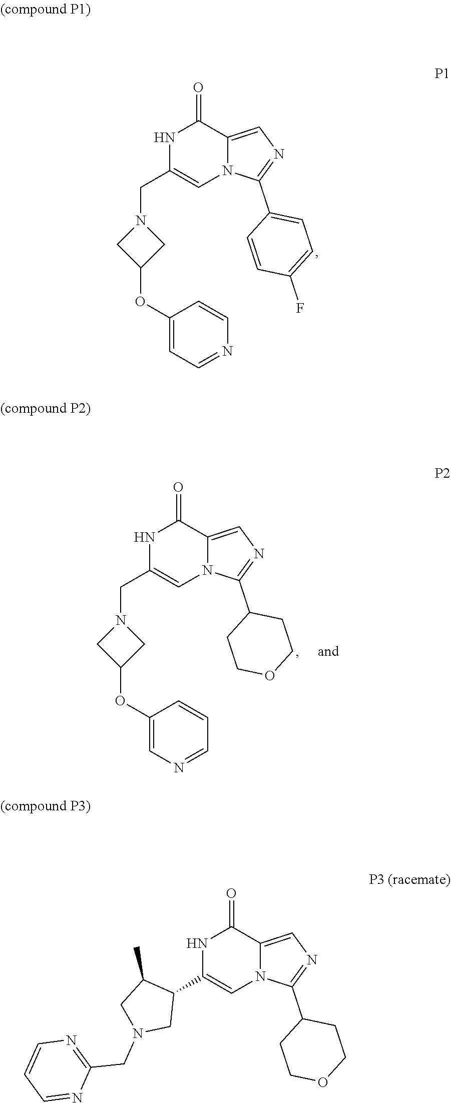 [SMILES: O=c1[nH]c(CN2CC(Oc3ccncc3)C2)cn2c(-c3ccc(F)cc3)ncc12 ; O=c1[nH]c(CN2CC(Oc3cccnc3)C2)cn2c(C3CCOCC3)ncc12 ; C[C@@H]1CN(Cc2ncccn2)C[C@H]1c1cn2c(C3CCOCC3)ncc2c(=O)[nH]1]

in racemic form and in enantiomerically enriched or pure form.

In an embodiment E2(E1) the enantiomerically pure variant of compound P3 is the first eluding compound when the racemic mixture of P3 is separated by Chiral HPLC (Column: Chiralpak IA, 250×4.6 mm×5 um; mobile phase Hex/EtOH/DEA=70:30:0.2) with a flow rate of 1.0 mL/min (P3 enantiomer 1).

E3(E1 and E2): A compound of any of E1 and E2 for the use as a medicament.

E4: A compound of any of E1 and E2 or the compound

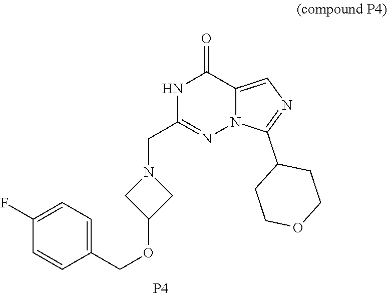

for use in the treatment of benign prostate hyperplasia or sickle cell disease.

E5: A pharmaceutical composition comprising a therapeutically effective amount of any of the compounds of E1 and E2 or the compound P4, and one or more pharmaceutically acceptable carriers, diluents or excipients.

E6(E5): The pharmaceutical is for the treatment of benign prostate hyperplasia or sickle cell disease.

E7: Use of the compound P4 or any of the compounds of E1 and E2 for the manufacture of a medicament for the treatment of benign prostate hyperplasia or sickle cell disease.

E8: A method of treating a subject suffering from benign prostate hyperplasia or sickle cell disease comprising administering a therapeutically effective amount of a compound P4 or any of the compounds of E1 and E2 to a subject in need thereof

E9: A compound selected from the group consisting of 3-(4-fluorophenyl)-6-((3-(pyridin-4-yloxy)azetidin-1-yl)methyl)imidazo[1,5-a]pyrazin-8(7H)-one (P1), 6-[3-(pyridin-3-yloxy)-azetidin-1-ylmethyl]-3-(tetrahydro-pyran-4-yl)-7H-imidazo[1,5-a]pyrazin-8-one (P2), 6-((3S, 4S)-4-methyl-1-pyrimidin-2-ylmethyl-pyrrolidin-3-yl)-3-(tetrahydro-pyran-4-yl)-7H-imidazo[1,5-a]pyrazin-8-one (P3, enantiomer 1, or P3.1), and 6-((3R, 4R)-4-methyl-1-pyrimidin-2-ylmethyl-pyrrolidin-3-yl)-3-(tetrahydro-pyran-4-yl)-7H-imidazo[1,5-a]pyrazin-8-one (P3, enantiomer 2).

E10(E9) The compound 6-((3S, 4S)-4-methyl-1-pyrimidin-2-ylmethyl-pyrrolidin-3-yl)-3-(tetrahydro-pyran-4-yl)-7H-imidazo[1,5-a]pyrazin-8-one (P3, enantiomer 1).

E11(E9) The compound 6-((3R, 4R)-4-methyl-1-pyrimidin-2-ylmethyl-pyrrolidin-3-yl)-3-(tetrahydro-pyran-4-yl)-7H-imidazo[1,5-a]pyrazin-8-one (P3, enantiomer 2).

E12 (E9, E10 and E11) A compound of any of E9 to E11 for the use as a medicament.

E13: A compound selected from the group consisting of 3-(4-fluorophenyl)-6-((3-(pyridin-4-yloxy)azetidin-1-yl)methyl)imidazo[1,5-a]pyrazin-8(7H)-one (P1), 6-[3-(pyridin-3-yloxy)-azetidin-1-ylmethyl]-3-(tetrahydro-pyran-4-yl)-7H-imidazo[1,5-a]pyrazin-8-one (P2), 6-((3S, 4S)-4-methyl-1-pyrimidin-2-ylmethyl-pyrrolidin-3-yl)-3-(tetrahydro-pyran-4-yl)-7H-imidazo[1,5-a]pyrazin-8-one (P3, enantiomer 1), 6-((3R, 4R)-4-methyl-1-pyrimidin-2-ylmethyl-pyrrolidin-3-yl)-3-(tetrahydro-pyran-4-yl)-7H-imidazo[1,5-a]pyrazin-8-one (P3, enantiomer 2) and 2-[3-(4-fluoro-phenoxy)-azetidin-1-ylmethyl]-7-(tetrahydro-pyran-4-yl)-3H-imidazo[5,1-f][1,2,4]triazin-4-one (P4) for use in the treatment of benign prostate hyperplasia or sickle cell disease.

E14: A pharmaceutical composition comprising a therapeutically effective amount of any of the compounds 3-(4-fluorophenyl)-6-((3-(pyridin-4-yloxy)azetidin-1-yl)methyl)imidazo[1,5-a]pyrazin-8(7H)-one (P1), 6-[3-(pyridin-3-yloxy)-azetidin-1-ylmethyl]-3-(tetrahydro-pyran-4-yl)-7H-imidazo[1,5-a]pyrazin-8-one (P2), 6-((3S, 4S)-4-methyl-1-pyrimidin-2-ylmethyl-pyrrolidin-3-yl)-3-(tetrahydro-pyran-4-yl)-7H-imidazo[1,5-a]pyrazin-8-one (P3, enantiomer 1), 6-((3R, 4R)-4-methyl-1-pyrimidin-2-ylmethyl-pyrrolidin-3-yl)-3-(tetrahydro-pyran-4-yl)-7H-imidazo[1,5-a]pyrazin-8-one (P3, enantiomer 2) and 2-[3-(4-fluoro-phenoxy)-azetidin-1-ylmethyl]-7-(tetrahydro-pyran-4-yl)-3H-imidazo[5,1-f][1,2,4]triazin-4-one (P4), and one or more pharmaceutically acceptable carriers, diluents or excipients

E15(E14): The pharmaceutical is for the treatment of benign prostate hyperplasia or sickle cell disease.

E16: Use of any of the compounds 3-(4-fluorophenyl)-6-((3-(pyridin-4-yloxy)azetidin-1-yl)methyl)imidazo[1,5-a]pyrazin-8(7H)-one (P1), 6-[3-(pyridin-3-yloxy)-azetidin-1-ylmethyl]-3-(tetrahydro-pyran-4-yl)-7H-imidazo[1,5-a]pyrazin-8-one (P2), 6-((3S, 4S)-4-methyl-1-pyrimidin-2-ylmethyl-pyrrolidin-3-yl)-3-(tetrahydro-pyran-4-yl)-7H-imidazo[1,5-a]pyrazin-8-one (P3, enantiomer 1), 6-((3R, 4R)-4-methyl-1-pyrimidin-2-ylmethyl-pyrrolidin-3-yl)-3-(tetrahydro-pyran-4-yl)-7H-imidazo[1,5-a]pyrazin-8-one (P3, enantiomer 2) and 2-[3-(4-fluoro-phenoxy)-azetidin-1-ylmethyl]-7-(tetrahydro-pyran-4-yl)-3H-imidazo[5,1-f][1,2,4]triazin-4-one (P4) for the manufacture of a medicament for the treatment of benign prostate hyperplasia or sickle cell disease.

E17: A method of treating a subject suffering from benign prostate hyperplasia or sickle cell disease comprising administering a therapeutically effective amount of any of the compounds 3-(4-fluorophenyl)-6-((3-(pyridin-4-yloxy)azetidin-1-yl)methyl)imidazo[1,5-a]pyrazin-8(7H)-one (P1), 6-[3-(Pyridin-3-yloxy)-azetidin-1-ylmethyl]-3-(tetrahydro-pyran-4-yl)-7H-imidazo[1,5-a]pyrazin-8-one (P2), 6-((3S, 4S)-4-methyl-1-pyrimidin-2-ylmethyl-pyrrolidin-3-yl)-3-(tetrahydro-pyran-4-yl)-7H-imidazo[1,5-a]pyrazin-8-one (P3, enantiomer 1), 6-((3R, 4R)-4-methyl-1-pyrimidin-2-ylmethyl-pyrrolidin-3-yl)-3-(tetrahydro-pyran-4-yl)-7H-imidazo[1,5-a]pyrazin-8-one (P3, enantiomer 2) and 2-[3-(4-fluoro-phenoxy)-azetidin-1-ylmethyl]-7-(tetrahydro-pyran-4-yl)-3H-imidazo[5,1-f][1,2,4]triazin-4-one (P4) to a subject in need thereof.

Table 1 lists compound examples of the invention and the corresponding IC50 values (nM) determined as described in the section “PDE9 inhibition assay”. Further, the concentration of compounds in plasma and brain, determined as described in the section “Blood Brain Barrier penetration”, are listed. Each of the compounds constitutes an individual embodiment of the present invention:

TABLE 1
Compound examples of the invention, IC50 values and plasma/brain
concentration

| | | | Plasma | Brain | |
|---|---|---|---|---|---|
| | | | concentration | concentration | Brain/Plasma |
| | PDE9 | PDE1 | after 30 minutes | after 30 minutes | ratio after 30 |
| | IC50 | IC50 | and 120 minutes | and 120 minutes | minutes and |
| Compound | (nM) | (nM) | (ng/mL) | (ng/mL) | 120 minutes |
| Compound (P1) | 42 | 45090 | 30 min.: 719 | 30 min.: 42 | 0.06 |
| | | | 120 min.: 86 | 120 min.: 7 | 0.08 |
| Compound (P2) | 36 | 5283 | 30 min.: 715 | Below detection | Not calculated |
| | | | 120 min.: 11 | limit | (brain |
| | | | | | concentration |
| | | | | | below limit of |
| | | | | | detection) |
| Compound (P3, | 49 | 3000 | 30 min.: 1620 | 30 min.: 67 | 0.04 |
| enantiomer 1) | | | 120 min.: 226 | 120 min.: 7 | 0.03 |
| Compound (P4) | 10 | 1009 | 30 min.: 3380 | 30 min.: 125 | 0.04 |
| | | | 120 min. 352 | 120 min.: 15 | 0.04 |
| 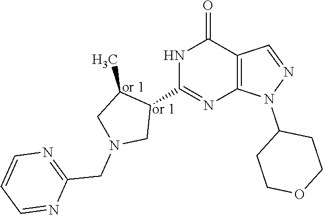 [SMILES: C[C@@H]1CN(Cc2ncccn2)C[C@H]1c1nc2c(cnn2C2CCOCC2)c(=O)[nH]1] Reference compound disclosed in WO2008/139293 (AF27873 or PF- 04447943) | 70 | 2500 | 30 min.: 1230 120 min.: 529 | 30 min.: 500 120 min.: 215 | 0.41 0.41 |

### II. Pharmaceutical Composition

The present invention further provides a pharmaceutical composition comprising a therapeutically effective amount of any of the compounds of the present invention and a pharmaceutically acceptable carrier or diluent. The present invention also provides a pharmaceutical composition comprising a therapeutically effective amount of one of the specific compounds disclosed herein and a pharmaceutically acceptable carrier or diluent.

### Pharmaceutically Acceptable Salts

The present invention also comprises salts of the compounds, typically, pharmaceutically acceptable salts. Such salts include pharmaceutically acceptable acid addition salts. Acid addition salts include salts of inorganic acids as well as organic acids.

Representative examples of suitable inorganic acids include hydrochloric, hydrobromic, hydroiodic, phosphoric, sulfuric, sulfamic, nitric acids and the like. Representative examples of suitable organic acids include formic, acetic, trichloroacetic, trifluoroacetic, propionic, benzoic, cinnamic, citric, fumaric, glycolic, itaconic, lactic, methanesulfonic, maleic, malic, malonic, mandelic, oxalic, picric, pyruvic, salicylic, succinic, methane sulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, theophylline acetic acids, as well as the 8-halotheophyllines, for example 8-bromotheophylline and the like. Further examples of pharmaceutically acceptable inorganic or organic acid addition salts include the pharmaceutically acceptable salts listed in Berge, S. M. et al., J. Pharm. Sci. 1977, 66, 2, the contents of which are hereby incorporated by reference.

Furthermore, the compounds of this invention may exist in unsolvated as well as in solvated forms with pharmaceutically acceptable solvents such as water, ethanol and the like. In general, the solvated forms are considered equivalent to the unsolvated forms for the purposes of this invention.

The compounds of the invention may be administered alone or in combination with pharmaceutically acceptable carriers, diluents or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 22nd Edition, Gennaro, Ed., Mack Publishing Co., Easton, Pa., 2013.

The pharmaceutical compositions may be specifically formulated for administration by any suitable route such as oral, rectal, nasal, pulmonary, topical (including buccal and sublingual), transdermal, intracisternal, intraperitoneal, vaginal and parenteral (including subcutaneous, intramuscular, intrathecal, intravenous and intradermal) routes. It will be appreciated that the route will depend on the general health and age of the subject to be treated, the nature of the condition to be treated and the active ingredient.

Pharmaceutical compositions for oral administration include solid dosage forms such as capsules, tablets, dragees, pills, lozenges, powders and granules. Where appropriate, the compositions may be prepared with coatings such as enteric coatings or they may be formulated so as to provide controlled release of the active ingredient such as sustained or prolonged release according to methods well known in the art. Liquid dosage forms for oral administration include solutions, emulsions, suspensions, syrups and elixirs.

Pharmaceutical compositions for parenteral administration include sterile aqueous and nonaqueous injectable solutions, dispersions, suspensions or emulsions as well as sterile powders to be reconstituted in sterile injectable solutions or dispersions prior to use. Other suitable administration forms include, but are not limited to, suppositories, sprays, ointments, creams, gels, inhalants, dermal patches and implants.

Typical oral dosages range from about 0.001 to about 100 mg/kg body weight per day. Typical oral dosages also range from about 0.01 to about 50 mg/kg body weight per day. Typical oral dosages further range from about 0.05 to about 10 mg/kg body weight per day. Oral dosages are usually administered in one or more dosages, typically, one to three dosages per day. The exact dosage will depend upon the frequency and mode of administration, the gender, age, weight and general health of the subject treated, the nature and severity of the condition treated and any concomitant diseases to be treated and other factors evident to those skilled in the art.

The formulations may also be presented in a unit dosage form by methods known to those skilled in the art. For illustrative purposes, a typical unit dosage form for oral administration may contain from about 0.01 to about 1000 mg, from about 0.05 to about 500 mg, or from about 0.5 mg to about 200 mg.

For parenteral routes such as intravenous, intrathecal, intramuscular and similar administration, typical doses are on the order of half the dose employed for oral administration.

The present invention also provides a process for making a pharmaceutical composition comprising admixing a therapeutically effective amount of a compound of the present invention and at least one pharmaceutically acceptable carrier or diluent. In an embodiment, of the present invention, the compound utilized in the aforementioned process is one of the specific compounds disclosed in the Experimental Section herein.

The compounds of this invention are generally utilized as the free substance or as a pharmaceutically acceptable salt thereof. Such salts are prepared in a conventional manner by treating a solution or suspension of a compound of the present invention with a molar equivalent of a pharmaceutically acceptable acid. Representative examples of suitable organic and inorganic acids are described above.

For parenteral administration, solutions of the compounds of the present invention in sterile aqueous solution, aqueous propylene glycol, aqueous vitamin E or sesame or peanut oil may be employed. Such aqueous solutions should be suitably buffered if necessary and the liquid diluent first rendered isotonic with sufficient saline or glucose.

The aqueous solutions are particularly suitable for intravenous, intramuscular, subcutaneous and intraperitoneal administration. The compounds of the present invention may be readily incorporated into known sterile aqueous media using standard techniques known to those skilled in the art.

Suitable pharmaceutical carriers include inert solid diluents or fillers, sterile aqueous solutions and various organic solvents. Examples of solid carriers include lactose, terra alba, sucrose, cyclodextrin, talc, gelatin, agar, pectin, acacia, magnesium stearate, stearic acid and lower alkyl ethers of cellulose. Examples of liquid carriers include, but are not limited to, syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax. The pharmaceutical compositions formed by combining the compounds of the present invention and a pharmaceutically acceptable carrier are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and optionally a suitable excipient. Furthermore, the orally available formulations may be in the form of a powder or granules, a solution or suspension in an aqueous or non-aqueous liquid, or an oil-in-water or water-in-oil liquid emulsion.

If a solid carrier is used for oral administration, the preparation may be tableted, placed in a hard gelatine capsule in powder or pellet form or it may be in the form of a troche or lozenge. The amount of solid carrier will vary widely but will range from about 25 mg to about 1 g per dosage unit. If a liquid carrier is used, the preparation may be in the form of a syrup, emulsion, soft gelatine capsule or sterile injectable liquid such as an aqueous or non-aqueous liquid suspension or solution.

The pharmaceutical compositions of the invention may be prepared by conventional methods in the art. For example, tablets may be prepared by mixing the active ingredient with ordinary adjuvants and/or diluents and subsequently compressing the mixture in a conventional tabletting machine prepare tablets. Examples of adjuvants or diluents comprise: corn starch, potato starch, talcum, magnesium stearate, gelatin, lactose, gums, and the like. Any other adjuvants or additives usually used for such purposes such as colorings, flavorings, preservatives etc. may be used provided that they are compatible with the active ingredients.

The pharmaceutical compositions may comprise at least 10%, 20%, 30%, 40%, 50%, 60%, 70%, 80%, or 90% by weight of PDE9 inhibitors of the present invention.

In one embodiment, the pharmaceutical composition comprising compounds of the present invention is used in combination with an additional active agent, such as HU. The compounds of the present invention and the additional active agent may be administered simultaneously, sequentially, or at any order. The compounds of the present invention and the additional active agent may be administered at different dosages, with different dosing frequencies, or via different routes, whichever is suitable.

The term “administered simultaneously”, as used herein, is not specifically restricted and means that the compounds of the present invention and the additional active agent are substantially administered at the same time, e.g. as a mixture or in immediate subsequent sequence.

The term “administered sequentially”, as used herein, is not specifically restricted and means that the compounds of the present invention and the additional active agent are not administered at the same time but one after the other, or in groups, with a specific time interval between administrations. The time interval may be the same or different between the respective administrations of the compounds of the present invention and the additional active agent and may be selected, for example, from the range of 2 minutes to 96 hours, 1 to 7 days or one, two or three weeks. Generally, the time interval between the administrations may be in the range of a few minutes to hours, such as in the range of 2 minutes to 72 hours, 30 minutes to 24 hours, or 1 to 12 hours. Further examples include time intervals in the range of 24 to 96 hours, 12 to 36 hours, 8 to 24 hours, and 6 to 12 hours.

The molar ratio of the compounds of the present invention and the additional active agent is not particularly restricted. For example, when the compounds of the present invention and one additional active agent are combined in a composition, the molar ratio of them may be in the range of 1:500 to 500:1, or of 1:100 to 100:1, or of 1:50 to 50:1, or of 1:20 to 20:1, or of 1:5 to 5:1, or 1:1. Similar molar ratios apply when the compounds of the present invention and two or more other active agents are combined in a composition. The compounds of the present invention compounds of the present invention may comprise a predetermined molar weight percentage from about 1% to 10%, or about 10% to about 20%, or about 20% to about 30%, or about 30% to 40%, or about 40% to 50%, or about 50% to 60%, or about 60% to 70%, or about 70% to 80%, or about 80% to 90%, or about 90% to 99% of the composition.

### III. Methods of Using Compounds of the Invention

PDE9 is expressed specifically in the human haematopoietic system including neutrophils, reticulocytes erythroid and erythroleukaemic cells. Furthermore, SCD patients exhibit a marked and significant elevation of PDE9 expression in reticulocytes and neutrophils compared to healthy individuals (Almeida et al., Br J Haematol. 2008 September; 142(5):836-44). Evidence additionally demonstrates a link between PDE9 and cell adhesion since pharmacologic PDE9 inhibition ameliorates the increased adhesive properties of SCD neutrophils (Miguel et al., Inflamm Res. 2011 July; 60(7):633-42). The mechanism by which PDE9 inhibition decreases cell adhesion has been shown to be mediated by increased cGMP and decreased endothelial adhesion molecule expression. Importantly, in an animal model of SCD, the PDE9 inhibitor-mediated decrease in cell adhesion had the functional effect of increased cell survival. In addition to demonstrating decreased cell adhesion comparable to HU, PDE9 inhibition resulted in increased fetal non-sickled haemoglobin (HbF) production, which reduced the cellular concentration of abnormal haemoglobin (HbS) within red blood cells (RBCs) resulting in less polymerization of the abnormal haemoglobin and its associated sequelae. The importance of increasing HbF in treating SCD is evidenced by results of large studies like the Cooperative Study of Sickle Cell Disease, as well as studies in a variety of patient cohorts outside of the US, showing that HbF is among the most important modifiers of this disease (Alsultan et al., Am J Hematol., 88(6):531-2 (2013)) as well as data showing that modifiers of HbF improve other hematological parameters (Akinsheye, Blood, 118(1):19-27 (2011)). Finally, Almeida and colleagues demonstrated that treatment with HU combined with PDE9 inhibition in a mouse model of SCD leads to an additional beneficial amplification of the cGMP elevating effects of HU (Almeida et al., Blood. 2012 Oct. 4; 120(14):2879-88). In conclusion, PDE9 inhibition can modulate both the expression of fetal haemoglobin production as well as decrease cell adhesion, both mechanisms key for the treatment of SCD.

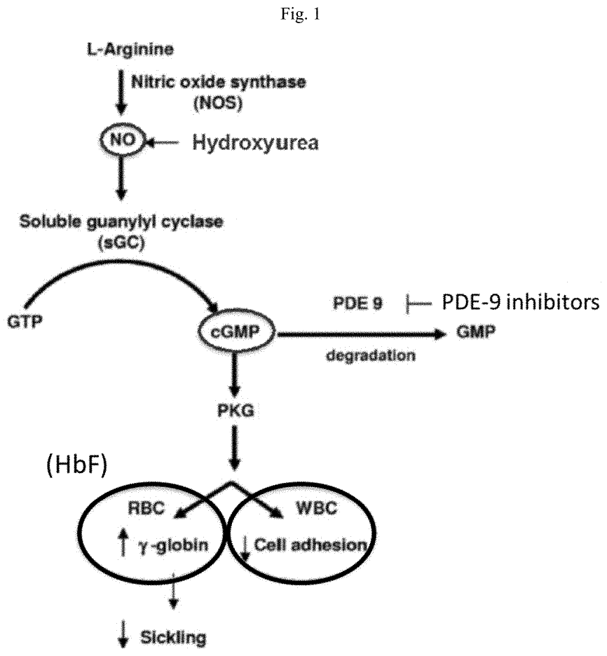

FIG. 1 is a graph showing PDE9 inhibitors of the present invention and hydroxyurea (HU) act through different mechanisms. HU increases nitric oxide (NO) levels, which activate soluble guanylyl cyclase (sGC) to generate cGMP. PDE9 inhibitors of the present invention block the degradation of cGMP by inhibiting PDE9 enzymatic activity, thus elevating cGMP levels. In erythroid lineages, cGMP binds to protein kinase G (PKG) and signals synthesis of fetal gamma globin and ultimately production of HbF. In hematopoietic cells where PDE9 expression is high, the direct inhibition of PDE9 activity increases cGMP levels, which promotes decreased leucocyte adhesion (figure modified from Almeida et al., Blood, vol. 120(14):2879-88 (2012)).

One aspect of the present invention provides methods of using PDE9 inhibitors of the present invention and pharmaceutical compositions comprising PDE9 inhibitors of the present invention.

PDE9 inhibitors of the present invention may be used to treat sickle cell disease or any disease and/or symptom related to sickle cell disease, such as anemia, sickle-hemoglobin C disease (SC), beta thalassemia (beta-plus thalassemia and beta-zero thalassemia), vaso-occlusive crisis, attacks of pain (sickle cell crisis), splenic sequestration crisis, acute chest syndrome, aplastic crisis, haemolytic crisis, long-term pain, bacterial infections, and stroke.

In one embodiment, PDE9 inhibitors of the present invention are used to treat beta thalassemia of a subject and/or to increase hemoglobin levels in the subject.

In another embodiment, PDE9 inhibitors of the present invention are used to increase cGMP levels in a cell or in the plasma of a subject, wherein the subject has sickle cell disease. The cell may be, but not limited to, red blood cells and/or white blood cells. The cGMP level may be increased by at least 50%, 100%, 150%, 2 times, 3 times, 4 times, 5 times, 10 times, 15 times, 20 times, or 25 times.

In another embodiment, PDE9 inhibitors of the present invention are used to increase fetal haemoglobin (HbF) positive red blood cell number in a subject, wherein the subject has sickle cell disease. The HbF positive red blood cell number is increased by at least 50%, 100%, 150%, 2 times, 3 times, 4 times, 5 times, 10 times, 15 times, 20 times, or 25 times.

In another embodiment, PDE9 inhibitors of the present invention are used to reduce sickle red blood cell percentage (% sickle RBC), stasis percentage (% stasis), total bilirubin, or total leucocyte count in a subject, wherein the subject has sickle cell disease. The % sickle RBC, % stasis, total bilirubin, total leucocyte count or spleen weight is decreased by at least 10%, 20%, 30%, 40%, 50%, 60% or 70%.

cGMP level may be measured with any suitable method in the art, such as enzyme immunoassay.

HbF positive cells, as used herein, means red blood cells with HbF. HbF positive cells may be measured from a blood sample with any suitable method in the art, such as electrophoresis and/or colorimetric methods.

Sickle red blood cells, sickled red blood cells, as used herein, means red blood cells with a crescent or sickle shape. % sickle red blood cell may be measured from a blood sample with any suitable method in the art.

Stasis or microvascular stasis, as used herein, is serious slowing, or complete cessation, of blood or lymph flow through vessels. % stasis is the number of static (no flow) venules divided by the number of flowing venules times 100. % stasis may be measured with any suitable method in the art.

Total bilirubin, as used herein, means both unconjugated and conjugated bilirubin. Total bilirubin levels may be measured from a blood sample with any suitable method in the art.

Total leucocyte count or total white blood cell count, as used herein, is a blood test that measures the number of white blood cells in the body. It may be measured from a blood sample with any suitable method in the art.

Another aspect of the present invention provides methods of using a PDE9 inhibitor of the present invention in combination with at least one other active agent. They may be administered simultaneously or sequentially. They may be present as a mixture for simultaneous administration, or may each be present in separate containers for sequential administration.

The term “simultaneous administration”, as used herein, is not specifically restricted and means that the PDE9 inhibitor of the present invention and the at least one other active agent are substantially administered at the same time, e.g. as a mixture or in immediate subsequent sequence.

The term “sequential administration”, as used herein, is not specifically restricted and means that the PDE9 inhibitor of the present invention and the at least one other active agent are not administered at the same time but one after the other, or in groups, with a specific time interval between administrations. The time interval may be the same or different between the respective administrations of PDE9 inhibitor of the present invention and the at least one other active agent and may be selected, for example, from the range of 2 minutes to 96 hours, 1 to 7 days or one, two or three weeks. Generally, the time interval between the administrations may be in the range of a few minutes to hours, such as in the range of 2 minutes to 72 hours, 30 minutes to 24 hours, or 1 to 12 hours. Further examples include time intervals in the range of 24 to 96 hours, 12 to 36 hours, 8 to 24 hours, and 6 to 12 hours.

The molar ratio of the PDE9 inhibitor of the present invention and the at least one other active agent is not particularly restricted. For example, when a PDE9 inhibitor of the present invention and one other active agent are combined in a composition, the molar ratio of them may be in the range of 1:500 to 500:1, or of 1:100 to 100:1, or of 1:50 to 50:1, or of 1:20 to 20:1, or of 1:5 to 5:1, or 1:1. Similar molar ratios apply when a PDE9 inhibitor of the present invention and two or more other active agent are combined in a composition. The PDE9 inhibitor of the present invention may comprise a predetermined molar weight percentage from about 1% to 10%, or about 10% to about 20%, or about 20% to about 30%, or about 30% to 40%, or about 40% to 50%, or about 50% to 60%, or about 60% to 70%, or about 70% to 80%, or about 80% to 90%, or about 90% to 99% of the composition.

The other active agent may be a different PDE9 inhibitor of the present invention or HU. The other active agent may also be an antibiotic agent such as penicillin, a nonsteroidal anti-inflammatory drug (NSAIDS) such as diclofenac or naproxen, a pain relief medication such as opioid, or folic acid.

Yet another aspect of the present invention provides methods of using a PDE9 inhibitor of the present invention in combination with at least one other therapy, such as but not limited to blood transfusion, bone marrow transplant, or gene therapy.

### IV. Kits and Devices

The invention provides a variety of kits and devices for conveniently and/or effectively carrying out methods of the present invention. Typically kits will comprise sufficient amounts and/or numbers of components to allow a user to perform multiple treatments of a subject(s) and/or to perform multiple experiments.

In one embodiment, the present invention provides kits for treating sickle cell disease, comprising a PDE9 inhibitor compound of the present invention or a combination of PDE9 inhibitor compounds of the present invention, optionally in combination with any other active agents, such as HU, an antibiotic agent such as penicillin, a nonsteroidal anti-inflammatory drug (NSAIDS) such as diclofenac or naproxen, a pain relief medication such as opioid, or folic acid.

The kit may further comprise packaging and instructions and/or a delivery agent to form a formulation composition. The delivery agent may comprise a saline, a buffered solution, or any delivery agent disclosed herein. The amount of each component may be varied to enable consistent, reproducible higher concentration saline or simple buffer formulations. The components may also be varied in order to increase the stability of PDE9 inhibitor compounds in the buffer solution over a period of time and/or under a variety of conditions.

The present invention provides for devices that may incorporate PDE9 inhibitor compounds of the present invention. These devices contain in a stable formulation available to be immediately delivered to a subject in need thereof, such as a human patient with sickle cell disease or beta thalassemia.

Non-limiting examples of the devices include a pump, a catheter, a needle, a transdermal patch, a pressurized olfactory delivery device, iontophoresis devices, multi-layered microfluidic devices. The devices may be employed to deliver PDE9 inhibitor compounds of the present invention according to single, multi- or split-dosing regiments. The devices may be employed to deliver PDE9 inhibitor compounds of the present invention across biological tissue, intradermal, subcutaneously, or intramuscularly. More examples of devices suitable for delivering PDE9 inhibitor compounds include but not limited to a medical device for intravesical drug delivery disclosed in International Publication WO 2014036555, a glass bottle made of type I glass disclosed in US Publication No. 20080108697, a drug-eluting device comprising a film made of a degradable polymer and an active agent as disclosed in US Publication No. 20140308336, an infusion device having an injection micropump, or a container containing a pharmaceutically stable preparation of an active agent as disclosed in U.S. Pat. No. 5,716,988, an implantable device comprising a reservoir and a channeled member in fluid communication with the reservoir as disclosed in International Publication WO 2015023557, a hollow-fibre-based biocompatible drug delivery device with one or more layers as disclosed in US Publication No. 20090220612, an implantable device for drug delivery including an elongated, flexible device having a housing defining a reservoir that contains a drug in solid or semi-solid form as disclosed in International Publication WO 2013170069, a bioresorbable implant device disclosed in U.S. Pat. No. 7,326,421, contents of each of which are incorporated herein by reference in their entirety.

### V. Definitions

The articles “a” and “an,” as used herein, should be understood to mean “at least one,” unless clearly indicated to the contrary.

The phrase “and/or,” as used herein, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements).

As used herein, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements.

In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, the phrase “at least one” in reference to a list of one or more elements should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, all transitional phrases such as “comprising,” “including,” “carrying,” “having,” “containing,” “involving,” “holding,” and the like are to be understood to be open-ended, i.e., to mean including but not limited to.

Only the transitional phrases “consisting of” and “consisting essentially of” shall be closed or semi-closed transitional phrases, respectively, as set forth in the United States Patent Office Manual of Patent Examining Procedures.

As used herein, a “subject” or a “patient” refers to any mammal (e.g., a human), such as a mammal that may be susceptible to a disease or disorder, for example, tumorigenesis or cancer. Examples include a human, a non-human primate, a cow, a horse, a pig, a sheep, a goat, a dog, a cat, or a rodent such as a mouse, a rat, a hamster, or a guinea pig. In various embodiments, a subject refers to one that has been or will be the object of treatment, observation, or experiment. For example, a subject can be a subject diagnosed with cancer or otherwise known to have cancer or one selected for treatment, observation, or experiment on the basis of a known cancer in the subject.

As used herein, “treatment” or “treating” refers to amelioration of a disease or disorder, or at least one sign or symptom thereof “Treatment” or “treating” can refer to reducing the progression of a disease or disorder, as determined by, e.g., stabilization of at least one sign or symptom or a reduction in the rate of progression as determined by a reduction in the rate of progression of at least one sign or symptom. In another embodiment, “treatment” or “treating” refers to delaying the onset of a disease or disorder.

As used herein, “prevention” or “preventing” refers to a reduction of the risk of acquiring or having a sign or symptom a given disease or disorder, i.e., prophylactic treatment.

The phrase “therapeutically effective amount” as used herein means that amount of a compound, material, or composition comprising a compound of the present teachings that is effective for producing a desired therapeutic effect. Accordingly, a therapeutically effective amount treats or prevents a disease or a disorder, e.g., ameliorates at least one sign or symptom of the disorder. In various embodiments, the disease or disorder is a cancer.

A dash (“-”) that is not between two letters or symbols is used to indicate a point of attachment for a substituent. For example, —CONH₂ is attached through the carbon atom (C).

By “optional” or “optionally,” it is meant that the subsequently described event or circumstance may or may not occur, and that the description includes instances where the event or circumstance occurs and instances in which it does not. For example, “optionally substituted aryl” encompasses both “aryl” and “substituted aryl” as defined herein. It will be understood by those ordinarily skilled in the art, with respect to any group containing one or more substituents, that such groups are not intended to introduce any substitution or substitution patterns that are sterically impractical, synthetically non-feasible, and/or inherently unstable.

The term “alkyl” as used herein refers to a saturated straight or branched hydrocarbon, such as a straight or branched group of 1-22, 1-8, 1-6, or 1-4 carbon atoms, referred to herein as (C₁-C₂₂)alkyl, (C₁-C₈)alkyl, (C₁-C₆)alkyl, and (C₁-C₄)alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, and octyl.

The term “alkenyl” as used herein refers to an unsaturated straight or branched hydrocarbon having at least one carbon-carbon double bond (shown, for example, as “=”), such as a straight or branched group of 2-22, 2-8, 2-6, or 2-4 carbon atoms, referred to herein as (C₂-C₂₂)alkenyl, (C₂-C₈)alkenyl, (C₂-C₆)alkenyl, and (C₂-C₄)alkenyl, respectively. Exemplary alkenyl groups include, but are not limited to, vinyl, allyl, butenyl, pentenyl, hexenyl, butadienyl, pentadienyl, hexadienyl, 2-ethylhexenyl, 2-propyl-2-butenyl, and 4-(2-methyl-3-butene)-pentenyl.

The term “alkynyl” as used herein refers to an unsaturated straight or branched hydrocarbon having at least one carbon-carbon triple bond (shown, for example, as “-”), such as a straight or branched group of 2-22, 2-8, 2-6, 2-4 carbon atoms, referred to herein as (C₂-C₂₂)alkynyl, (C₂-C₈)alkynyl, (C₂-C₆)alkynyl, and (C₂-C₄)alkynyl, respectively. Exemplary alkynyl groups include, but are not limited to, ethynyl, propynyl, butynyl, pentynyl, hexynyl, methylpropynyl, 4-methyl-1-butynyl, 4-propyl-2-pentynyl, and 4-butyl-2-hexynyl.

The term “cycloalkyl” as used herein refers to a saturated or unsaturated monocyclic, bicyclic, other multicyclic, or bridged cyclic hydrocarbon group. A cyclocalkyl group can have 3-22, 3-12, or 3-8 ring carbons, referred to herein as (C₃-C₂₂)cycloalkyl, (C₃-C₂)cycloalkyl, or (C3-C8)cycloalkyl, respectively. A cycloalkyl group can also have one or more carbon-carbon double bond or carbon-carbon triple bond.

Exemplary monocyclic cycloalkyl groups include, but are not limited to, cyclopentanes (cyclopentyls), cyclopentenes (cyclopentenyls), cyclohexanes (cyclohexyls), cyclohexenes (cyclopexenyls), cycloheptanes (cycloheptyls), cycloheptenes (cycloheptenyls), cyclooctanes (cyclooctyls), cyclooctenes (cyclooctenyls), cyclononanes (cyclononyls), cyclononenes (cyclononenyls), cyclodecanes (cyclodecyls), cyclodecenes (cyclodecenyls), cycloundecanes (cycloundecyls), cycloundecenes (cycloundecenyls), cyclododecanes (cyclododecyls), and cyclododecenes (cyclododecenyls). Other exemplary cycloalkyl groups, including bicyclic, multicyclic, and bridged cyclic groups, include, but are not limited to, bicyclobutanes (bicyclobutyls), bicyclopentanes (bicyclopentyls), bicyclohexanes (bicyclohexyls), bicycleheptanes (bicycloheptyls, including bicyclo[2,2,1]heptanes (bicycle[2,2,1]heptyls) and bicycle[3,2,0]heptanes (bicycle[3,2,0]heptyls)), bicyclooctanes (bicyclooctyls, including octahydropentalene (octahydropentalenyl), bicycle[3,2,1]octane (bicycle[3,2,1]octyl), and bicylo[2,2,2]octane (bicycle[2,2,2]octyl)), and adamantanes (adamantyls). Cycloalkyl groups can be fused to other cycloalkyl saturated or unsaturated, aryl, or heterocyclyl groups.

The term “aryl” as used herein refers to a mono-, bi-, or other multi-carbocyclic aromatic ring system. The aryl can have 6-22, 6-18, 6-14, or 6-10 carbons, referred to herein as (C₆-C₂₂)aryl, (C₆-C₁₈)aryl, (C₆-C₁₄)aryl, or (C₆-C₁₀)aryl, respectively. The aryl group can optionally be fused to one or more rings selected from aryls, cycloalkyls, and heterocyclyls. The term “bicyclic aryl” as used herein refers to an aryl group fused to another aromatic or non-aromatic carbocylic or heterocyclic ring. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Exemplary aryl groups also include, but are not limited to a monocyclic aromatic ring system, wherein the ring comprises 6 carbon atoms, referred to herein as “(C₆)aryl” or phenyl. The phenyl group can also be fused to a cyclohexane or cyclopentane ring to form another aryl.

The term “arylalkyl” as used herein refers to an alkyl group having at least one aryl substituent (e.g., -aryl-alkyl-). Exemplary arylalkyl groups include, but are not limited to, arylalkyls having a monocyclic aromatic ring system, wherein the ring comprises 6 carbon atoms, referred to herein as “(C₆)arylalkyl.” The term “benzyl” as used herein refers to the group —CH₂-phenyl.

The term “heteroalkyl” refers to an alkyl group as described herein in which one or more carbon atoms is replaced by a heteroatom. Suitable heteroatoms include oxygen, sulfur, nitrogen, phosphorus, and the like. Examples of heteroalkyl groups include, but are not limited to, alkoxy, amino, thioester, and the like.

The terms “heteroalkenyl” and “heteroalkynyl” refer to unsaturated aliphatic groups analogous in length and possible substitution to the heteroalkyls described above, but that contain at least one double or triple bond, respectively.

The term “heterocycle” refers to cyclic groups containing at least one heteroatom as a ring atom, in some cases, 1 to 3 heteroatoms as ring atoms, with the remainder of the ring atoms being carbon atoms. Suitable heteroatoms include oxygen, sulfur, nitrogen, phosphorus, and the like. In some cases, the heterocycle may be 3- to 10-membered ring structures or 3- to 7-membered rings, whose ring structures include one to four heteroatoms. The term “heterocycle” may include heteroaryl groups, saturated heterocycles (e.g., cycloheteroalkyl) groups, or combinations thereof. The heterocycle may be a saturated molecule, or may comprise one or more double bonds. In some case, the heterocycle is a nitrogen heterocycle, wherein at least one ring comprises at least one nitrogen ring atom. The heterocycles may be fused to other rings to form a polycylic heterocycle. Thus, heterocycles also include bicyclic, tricyclic, and tetracyclic groups in which any of the above heterocyclic rings is fused to one or two rings independently selected from aryls, cycloalkyls, and heterocycles. The heterocycle may also be fused to a spirocyclic group.

Heterocycles include, for example, thiophene, benzothiophene, thianthrene, furan, tetrahydrofuran, pyran, isobenzofuran, chromene, xanthene, phenoxathiin, pyrrole, dihydropyrrole, pyrrolidine, imidazole, pyrazole, pyrazine, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, triazole, tetrazole, oxazole, isoxazole, thiazole, isothiazole, phenanthridine, acridine, pyrimidine, phenanthroline, phenazine, phenarsazine, phenothiazine, furazan, phenoxazine, pyrrolidine, oxolane, thiolane, oxazole, oxazine, piperidine, homopiperidine (hexamethyleneimine), piperazine (e.g., N-methyl piperazine), morpholine, lactones, lactams such as azetidinones and pyrrolidinones, sultams, sultones, other saturated and/or unsaturated derivatives thereof, and the like.

In some cases, the heterocycle may be bonded to a compound via a heteroatom ring atom (e.g., nitrogen). In some cases, the heterocycle may be bonded to a compound via a carbon ring atom. In some cases, the heterocycle is pyridine, imidazole, pyrazine, pyrimidine, pyridazine, acridine, acridin-9-amine, bipyridine, naphthyridine, quinoline, isoquinoline, benzoquinoline, benzoisoquinoline, phenanthridine-1,9-diamine, or the like.

The term “heteroaromatic” or “heteroaryl” as used herein refers to a mono-, bi-, or multi-cyclic aromatic ring system containing one or more heteroatoms, for example 1-3 heteroatoms, such as nitrogen, oxygen, and sulfur. Heteroaryls can also be fused to non-aromatic rings. In various embodiments, the term “heteroaromatic” or “heteroaryl,” as used herein except where noted, represents a stable 5- to 7-membered monocyclic, stable 9- to 10-membered fused bicyclic, or stable 12- to 14-membered fused tricyclic heterocyclic ring system which contains an aromatic ring that contains at least one heteroatom selected from the group consisting of N, O, and S. In some embodiments, at least one nitrogen is in the aromatic ring.

Heteroaromatics or heteroaryls can include, but are not limited to, a monocyclic aromatic ring, wherein the ring comprises 2-5 carbon atoms and 1-3 heteroatoms, referred to herein as “(C₂-C₅)heteroaryl.” Illustrative examples of monocyclic heteroaromatic (or heteroaryl) include, but are not limited to, pyridine (pyridinyl), pyridazine (pyridazinyl), pyrimidine (pyrimidyl), pyrazine (pyrazyl), triazine (triazinyl), pyrrole (pyrrolyl), pyrazole (pyrazolyl), imidazole (imidazolyl), (1,2,3)- and (1,2,4)-triazole ((1,2,3)- and (1,2,4)-triazolyl), pyrazine (pyrazinyl), pyrimidine (pyrimidinyl), tetrazole (tetrazolyl), furan (furyl), thiophene (thienyl), isoxazole (isoxazolyl), thiazole (thiazolyl), isoxazole (isoxazolyl), and oxazole (oxazolyl).

The term “bicyclic heteroaromatic” or “bicyclic heteroaryl” as used herein refers to a heteroaryl group fused to another aromatic or non-aromatic carbocylic or heterocyclic ring. Exemplary bicyclic heteroaromatics or heteroaryls include, but are not limited to 5,6- or 6,6-fused systems, wherein one or both rings contain heteroatoms. The term “bicyclic heteroaromatic” or “bicyclic heteroaryl” also encompasses reduced or partly reduced forms of fused aromatic system wherein one or both rings contain ring heteroatoms. The ring system may contain up to three heteroatoms, independently selected from oxygen, nitrogen, and sulfur.

Exemplary bicyclic heteroaromatics (or heteroaryls) include, but are not limited to, quinazoline (quinazolinyl), benzoxazole (benzoxazolyl), benzothiophene (benzothiophenyl), benzoxazole (benzoxazolyl), benzisoxazole (benzisoxazolyl), benzimidazole (benzimidazolyl), benzothiazole (benzothiazolyl), benzofurane (benzofuranyl), benzisothiazole (benzisothiazolyl), indole (indolyl), indazole (indazolyl), indolizine (indolizinyl), quinoline (quinolinyl), isoquinoline (isoquinolinyl), naphthyridine (naphthyridyl), phthalazine (phthalazinyl), phthalazine (phthalazinyl), pteridine (pteridinyl), purine (purinyl), benzotriazole (benzotriazolyl), and benzofurane (benzofuranyl). In some embodiments, the bicyclic heteroaromatic (or bicyclic heteroaryl) is selected from quinazoline (quinazolinyl), benzimidazole (benzimidazolyl), benzothiazole (benzothiazolyl), indole (indolyl), quinoline (quinolinyl), isoquinoline (isoquinolinyl), and phthalazine (phthalazinyl). In certain embodiments, the bicyclic heteroaromatic (or bicyclic heteroaryl) is quinoline (quinolinyl) or isoquinoline (isoquinolinyl).

The term “tricyclic heteroaromatic” or “tricyclic heteroaryl” as used herein refers to a bicyclic heteroaryl group fused to another aromatic or non-aromatic carbocylic or heterocyclic ring. The term “tricyclic heteroaromatic” or “tricyclic heteroaryl” also encompasses reduced or partly reduced forms of fused aromatic system wherein one or both rings contain ring heteroatoms. Each of the rings in the tricyclic heteroaromatic (tricyclic heteroaryl) may contain up to three heteroatoms, independently selected from oxygen, nitrogen, and sulfur.

Exemplary tricyclic heteroaromatics (or heteroaryls) include, but are not limited to, acridine (acridinyl), 9H-pyrido[3,4-b]indole (9H-pyrido[3,4-b]indolyl), phenanthridine (phenanthridinyl), pyrido[1,2-a]benzimidazole (pyrido[1,2-a]benzimidazolyl), and pyrido[1,2-b]indazole (pyrido[1,2-b]indazolyl).

The term “alkoxy” as used herein refers to an alkyl group attached to an oxygen (—O-alkyl-). “Alkoxy” groups also include an alkenyl group attached to an oxygen (“alkenyloxy”) or an alkynyl group attached to an oxygen (“alkynyloxy”) groups. Exemplary alkoxy groups include, but are not limited to, groups with an alkyl, alkenyl or alkynyl group of 1-22, 1-8, or 1-6 carbon atoms, referred to herein as (C₁-C₂₂)alkoxy, (C₁-C₈)alkoxy, or (C₁-C₆)alkoxy, respectively. Exemplary alkoxy groups include, but are not limited to methoxy and ethoxy.

The term “cycloalkoxy” as used herein refers to a cycloalkyl group attached to an oxygen.

The term “aryloxy” or “aroxy” as used herein refers to an aryl group attached to an oxygen atom. Exemplary aryloxy groups include, but are not limited to, aryloxys having a monocyclic aromatic ring system, wherein the ring comprises 6 carbon atoms, referred to herein as “(C₆)aryloxy.” The term “arylalkoxy” as used herein refers to an arylalkyl group attached to an oxygen atom. An exemplary aryalkyl group is benzyloxy group.

The term “amine” or “amino” as used herein refers to both unsubstituted and substituted amines, e.g., NRₐR_{b}R_{b′}, where Rₐ, R_{b}, and R_{b′} are independently selected from alkyl, alkenyl, alkynyl, aryl, arylalkyl, carbamate, cycloalkyl, haloalkyl, heteroaryl, heterocyclyl, and hydrogen, and at least one of the Rₐ, R_{b}, and R_{b′} is not hydrogen. The amine or amino can be attached to the parent molecular group through the nitrogen. The amine or amino also may be cyclic, for example any two of Rₐ, R_{b}, and R_{b′} may be joined together and/or with the N to form a 3- to 12-membered ring (e.g., morpholino or piperidinyl). The term amino also includes the corresponding quaternary ammonium salt of any amino group. Exemplary amines include alkylamine, wherein at least one of Rₐ R_{b}, or R_{b′} is an alkyl group, or cycloalkylamine, wherein at least one of Rₐ R_{b}, or R_{b′} is a cycloalkyl group.

The term “ammonia” as used herein refers to NH₃.

The term “aldehyde” or “formyl” as used herein refers to —CHO.

The term “acyl” term as used herein refers to a carbonyl radical attached to an alkyl, alkenyl, alkynyl, cycloalkyl, heterocycyl, aryl, or heteroaryl. Exemplary acyl groups include, but are not limited to, acetyl, formyl, propionyl, benzoyl, and the like.

The term “amide” as used herein refers to the form —NRC(O)(R_{d})— or —C(O)NR_{c}Rₑ, wherein R_{c}, R_{d}, and Rₑ are each independently selected from alkyl, alkenyl, alkynyl, aryl, arylalkyl, cycloalkyl, haloalkyl, heteroaryl, heterocyclyl, and hydrogen. The amide can be attached to another group through the carbon, the nitrogen, R_{c}, R_{d}, or Rₑ. The amide also may be cyclic, for example R_{c} and Rₑ, may be joined to form a 3- to 12-membered ring, such as a 3- to 10-membered ring or a 5- or 6-membered ring. The term “amide” encompasses groups such as sulfonamide, urea, ureido, carbamate, carbamic acid, and cyclic versions thereof. The term “amide” also encompasses an amide group attached to a carboxy group, e.g., -amide-COOH or salts such as -amide-COONa.

The term “arylthio” as used herein refers to an aryl group attached to an sulfur atom. Exemplary arylthio groups include, but are not limited to, arylthios having a monocyclic aromatic ring system, wherein the ring comprises 6 carbon atoms, referred to herein as “(C₆)arylthio.”

The term “arylsulfonyl” as used herein refers to an aryl group attached to a sulfonyl group, e.g., —S(O)₂-aryl-. Exemplary arylsulfonyl groups include, but are not limited to, arylsulfonyls having a monocyclic aromatic ring system, wherein the ring comprises 6 carbon atoms, referred to herein as “(C₆)arylsulfonyl.”

The term “carbamate” as used herein refers to the form —R_{f}OC(O)N(R_{g})—, —R_{f}OC(O)N(R_{g})Rₕ—, or —OC(O)NR_{g}Rₕ, wherein R_{f}, R_{g}, and Rₕ are each independently selected from alkyl, alkenyl, alkynyl, aryl, arylalkyl, cycloalkyl, haloalkyl, heteroaryl, heterocyclyl, and hydrogen. Exemplary carbamates include, but are not limited to, arylcarbamates or heteroaryl carbamates (e.g., wherein at least one of R_{f}, R_{g} and Rₕ are independently selected from aryl or heteroaryl, such as pyridinyl, pyridazinyl, pyrimidinyl, and pyrazinyl).

The term “carbonyl” as used herein refers to —C(O)—.

The term “carboxy” or “carboxylate” as used herein refers to Rⱼ—COOH or its corresponding carboxylate salts (e.g., Rⱼ—COONa), where Rⱼ can independently be selected from alkoxy, aryloxy, alkyl, alkenyl, alkynyl, amide, amino, aryl, arylalkyl, cycloalkyl, ether, haloalkyl, heteroaryl, and heterocyclyl. Exemplary carboxys include, but are not limited to, alkyl carboxy wherein Rⱼ is alkyl, such as —O—C(O)-alkyl. Exemplary carboxy also include aryl or heteoraryl carboxy, e.g. wherein Rⱼ is an aryl, such as phenyl and tolyl, or heteroaryl group such as pyridine, pyridazine, pyrmidine and pyrazine. The term carboxy also includes “carboxycarbonyl,” e.g. a carboxy group attached to a carbonyl group, e.g., —C(O)—COOH or salts, such as —C(O)—COONa.

The term “dicarboxylic acid” as used herein refers to a group containing at least two carboxylic acid groups such as saturated and unsaturated hydrocarbon dicarboxylic acids and salts thereof. Exemplary dicarboxylic acids include alkyl dicarboxylic acids. Dicarboxylic acids include, but are not limited to succinic acid, glutaric acid, adipic acid, suberic acid, sebacic acid, azelaic acid, maleic acid, phthalic acid, aspartic acid, glutamic acid, malonic acid, fumaric acid, (+)/(−)-malic acid, (+)/(−) tartaric acid, isophthalic acid, and terephthalic acid. Dicarboxylic acids further include carboxylic acid derivatives thereof, such as anhydrides, imides, hydrazides (for example, succinic anhydride and succinimide).

The term “cyano” as used herein refers to —CN.

The term “ester” refers to the structure —C(O)O—, —C(O)O—Rᵢ—, —RⱼC(O)O—Rᵢ—, or —RⱼC(O)O—, where O is not bound to hydrogen, and Rᵢ and Rⱼ can independently be selected from alkoxy, aryloxy, alkyl, alkenyl, alkynyl, amide, amino, aryl, arylalkyl, cycloalkyl, ether, haloalkyl, heteroaryl, and heterocyclyl. Rᵢ can be a hydrogen, but Rⱼ cannot be hydrogen. The ester may be cyclic, for example the carbon atom and Rⱼ, the oxygen atom and Rᵢ, or Rᵢ and Rⱼ may be joined to form a 3- to 12-membered ring. Exemplary esters include, but are not limited to, alkyl esters wherein at least one of Rᵢ or Rⱼ is alkyl, such as —O—C(O)-alkyl, —C(O)—O-alkyl-, and -alkyl-C(O)—O-alkyl-. Exemplary esters also include aryl or heteroaryl esters, e.g. wherein at least one of Rᵢ or Rⱼ is an aryl group, such as phenyl or tolyl, or a heteroaryl group, such as pyridine, pyridazine, pyrimidine or pyrazine, such as a nicotinate ester. Exemplary esters also include reverse esters having the structure —RⱼC(O)O—, where the oxygen is bound to the parent molecule. Exemplary reverse esters include succinate, D-argininate, L-argininate, L-lysinate and D-lysinate. Esters also include carboxylic acid anhydrides and acid halides.

The term “ether” refers to the structure —RₖO—Rₗ—, where Rₖ and Rₗ can independently be alkyl, alkenyl, alkynyl, aryl, cycloalkyl, heterocyclyl, and ether. The ether can be attached to the parent molecular group through Rₖ or Rₗ. Exemplary ethers include, but are not limited to, alkoxyalkyl and alkoxyaryl groups. Ethers also include polyethers, e.g., where one or both of Rₖ and Rₗ are ethers.

The terms “halo” or “halogen” or “hal” or “halide” as used herein refer to F, Cl, Br, or I.

The term “haloalkyl” as used herein refers to an alkyl group substituted with one or more halogen atoms. “Haloalkyls” also encompass alkenyl or alkynyl groups substituted with one or more halogen atoms.

The terms “hydroxy” and “hydroxyl” as used herein refers to —OH.

The term “hydroxyalkyl” as used herein refers to a hydroxy attached to an alkyl group.

The term “hydroxyaryl” as used herein refers to a hydroxy attached to an aryl group.

The term “ketone” as used herein refers to the structure —C(O)—Rₘ (such as acetyl, —C(O)CH₃) or —RₘC(O)—Rₙ—. The ketone can be attached to another group through Rₘ or Rₙ. Rₘ or Rₙ can be alkyl, alkenyl, alkynyl, cycloalkyl, heterocyclyl or aryl, or Rₘ or Rₙ can be joined to form, for example, a 3- to 12-membered ring.

The term “monoester” as used herein refers to an analogue of a dicarboxylic acid wherein one of the carboxylic acids is functionalized as an ester and the other carboxylic acid is a free carboxylic acid or salt of a carboxylic acid. Examples of monoesters include, but are not limited to, to monoesters of succinic acid, glutaric acid, adipic acid, suberic acid, sebacic acid, azelaic acid, oxalic and maleic acid.

The term “nitro” as used herein refers to —NO₂.

The term “nitrate” as used herein refers to NO₃⁻.

The term “perfluoroalkyl” as used herein refers to an alkyl group in which all of the hydrogen atoms have been replaced by fluorine atoms. Exemplary perfluoroalkyl groups include, but are not limited to, C₁-C₅ perfluoroalkyl, such as trifluoromethyl.

The term “perfluorocycloalkyl” as used herein refers to a cycloalkyl group in which all of the hydrogen atoms have been replaced by fluorine atoms.

The term “perfluoroalkoxy” as used herein refers to an alkoxy group in which all of the hydrogen atoms have been replaced by fluorine atoms.

The term “phosphate” as used herein refers to the structure —OP(O)O₂²⁻, —RₒOP(O)O₂²⁻, —OP(O)(OR_{q})O⁻, or —RₒOP(O)(ORₚ)O⁻, wherein Rₒ, Rₚ and R_{q} each independently can be alkyl, alkenyl, alkynyl, aryl, cycloalkyl, heterocyclyl, or hydrogen.

The term “sulfide” as used herein refers to the structure —R_{q}S—, where R_{q} can be alkyl, alkenyl, alkynyl, aryl, arylalkyl, cycloalkyl, haloalkyl, heteroaryl, heterocyclyl. The sulfide may be cyclic, for example, forming a 3 to 12-membered ring. The term “alkylsulfide” as used herein refers to an alkyl group attached to a sulfur atom.

The term “sulfinyl” as used herein refers to the structure —S(O)O—, —RᵣS(O)O—, —RᵣS(O)ORₛ—, or —S(O)ORₛ—, wherein Rᵣ and Rₛ can be alkyl, alkenyl, aryl, arylalkyl, cycloalkyl, haloalkyl, heteroaryl, heterocyclyl, hydroxyl. Exemplary sulfinyl groups include, but are not limited to, alkylsulfinyls wherein at least one of Rᵣ or Rₛ is alkyl, alkenyl, or alkynyl.

The term “sulfonamide” as used herein refers to the structure —(Rₜ)—N—S(O)₂—Rᵥ— or —Rₜ(Rᵤ)N—S(O)₂—Rᵥ, where Rₜ, Rᵤ, and Rᵥ can be, for example, hydrogen, alkyl, alkenyl, alkynyl, aryl, cycloalkyl, and heterocyclyl. Exemplary sulfonamides include alkylsulfonamides (e.g., where Rᵥ is alkyl), arylsulfonamides (e.g., where Rᵥ is aryl), cycloalkyl sulfonamides (e.g., where Rᵥ is cycloalkyl), and heterocyclyl sulfonamides (e.g., where R, is heterocyclyl).

The term “sulfonate” as used herein refers to a salt or ester of a sulfonic acid. The term “sulfonic acid” refers to R_{w}SO₃H, where R_{w} is alkyl, alkenyl, alkynyl, aryl, cycloalkyl, or heterocyclyl (e.g., alkylsulfonyl). The term “sulfonyl” as used herein refers to the structure RₓSO₂—, where Rₓ can be alkyl, alkenyl, alkynyl, aryl, cycloalkyl, and heterocyclyl (e.g., alkylsulfonyl). The term “alkylsulfonyl” as used herein refers to an alkyl group attached to a sulfonyl group. “Alkylsulfonyl” groups can optionally contain alkenyl or alkynyl groups.

The term “sulfonate” as used herein refers R_{w}SO₃⁻, where R_{w} is alkyl, alkenyl, alkynyl, cycloalkyl, aryl, heterocyclyl, hydroxyl, alkoxy, aroxy, or aralkoxy, where each of the alkyl, alkenyl, alkynyl, cycloalkyl, aryl, heteroaryl, alkoxy, aroxy, or aralkoxy optionally is substituted. Non-limiting examples include triflate (also known as trifluoromethanesulfonate, CF₃SO₃⁻), benzenesulfonate, tosylate (also known as toluenesulfonate), and the like.

The term “thioketone” refers to the structure —R_{y}—C(S)—R_{z}—. The ketone can be attached to another group through R_{y} or R_{z}. R_{y} or R_{z} can be alkyl, alkenyl, alkynyl, cycloalkyl, heterocyclyl or aryl, or R_{y} or R_{z} can be joined to form a ring, for example, a 3- to 12-membered ring.

Each of the above groups may be optionally substituted. As used herein, the term “substituted” is contemplated to include all permissible substituents of organic compounds, “permissible” being in the context of the chemical rules of valence known to those of ordinary skill in the art. It will be understood that “substituted” also includes that the substitution results in a stable compound, e.g., which does not spontaneously undergo transformation such as by rearrangement, cyclization, elimination, etc. In some cases, “substituted” may generally refer to replacement of a hydrogen with a substituent as described herein. However, “substituted,” as used herein, does not encompass replacement and/or alteration of a functional group by which a molecule is identified, e.g., such that the “substituted” functional group becomes, through substitution, a different functional group. For example, a “substituted phenyl group” must still comprise the phenyl moiety and cannot be modified by substitution, in this definition, to become, e.g., a pyridine ring.

In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. For purposes of the present teachings, the heteroatoms such as nitrogen may have hydrogen substituents and/or any permissible substituents of organic compounds described herein which satisfy the valencies of the heteroatoms.

In various embodiments, the substituent is selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone, each of which optionally is substituted with one or more suitable substituents. In some embodiments, the substituent is selected from alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone, wherein each of the alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone can be further substituted with one or more suitable substituents.

Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, thioketone, ester, heterocyclyl, —CN, aryl, aryloxy, perhaloalkoxy, aralkoxy, heteroaryl, heteroaryloxy, heteroarylalkyl, heteroaralkoxy, azido, alkylthio, oxo, acylalkyl, carboxy esters, carboxamido, acyloxy, aminoalkyl, alkylaminoaryl, alkylaryl, alkylaminoalkyl, alkoxyaryl, arylamino, aralkylamino, alkylsulfonyl, carboxamidoalkylaryl, carboxamidoaryl, hydroxyalkyl, haloalkyl, alkylaminoalkylcarboxy, aminocarboxamidoalkyl, cyano, alkoxyalkyl, perhaloalkyl, arylalkyloxyalkyl, and the like. In some embodiments, the substituent is selected from cyano, halogen, hydroxyl, and nitro.

As a non-limiting example, in various embodiments when one of the Rₐ, R_{b}, and R_{b′} in NRₐR_{b}R_{b′}, referred to herein as an amine or amino, is selected from alkyl, alkenyl, alkynyl, cycloalkyl, and heterocyclyl, each of the alkyl, alkenyl, alkynyl, cycloalkyl, and heterocyclyl independently can be optionally substituted with one or more substituents each independently selected from alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone, wherein each of the alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone can be further substituted with one or more suitable substituents. In some embodiments when the amine is an alkyl amine or a cycloalkylamine, the alkyl or the cycloalkyl can be substituted with one or more substituents each independently selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone. In certain embodiments when the amine is an alkyl amine or a cycloalkylamine, the alkyl or the cycloalkyl can be substituted with one or more substituents each independently selected from amino, carboxy, cyano, and hydroxyl. For example, the alkyl or the cycloalkyl in the alkyl amine or the cycloalkylamine is substituted with an amino group, forming a diamine.

As used herein, a “suitable substituent” refers to a group that does not nullify the synthetic or pharmaceutical utility of the compounds of the invention or the intermediates useful for preparing them. Examples of suitable substituents include, but are not limited to: (C₁-C₂₂), (C₁-C₈), (C₁-C₆), or (C₁-C₄) alkyl, alkenyl or alkynyl; (C₆-C₂₂), (C₆-C₁₈), (C₆-C₁₄), or (C₆-C₁₀) aryl; (C₂-C₂₁), (C₂-C₁₇), (C₂-C₁₃), or (C₂-C₉) heteroaryl; (C₃-C₂₂), (C₃-C₁₂), or (C₃-C₈) cycloalkyl; (C₁-C₂₂), (C₁-C₈), (C₁-C₆), or (C₁-C₄) alkoxy; (C₆-C₂₂), (C₆-C₁₈), (C₆-C₁₄), or (C₆-C₁₀) aryloxy; —CN; —OH; oxo; halo; carboxy; amino, such as —NH((C₁-C₂₂), (C₁-C₈), (C₁-C₆), or (C₁-C₄) alkyl), —N((C₁-C₂₂), (C₁-C₈), (C₁-C₆), or (C₁-C₄) alkyl)₂, —NH((C₆)aryl), or —N((C₆-C₁₀) aryl)₂; formyl; ketones, such as —CO((C₁-C₂₂), (C₁-C₈), (C₁-C₆), or (C₁-C₄) alkyl), —CO(((C₆-C₁₀) aryl) esters, such as —CO₂((C₁-C₂₂), (C₁-C₈), (C₁-C₆), or (C₁-C₄) alkyl) and —CO₂((C₆-C₁₀) aryl). One of skill in art can readily choose a suitable substituent based on the stability and pharmacological and synthetic activity of the compound of the invention.

Unless otherwise specified, the chemical groups include their corresponding monovalent, divalent, trivalent, and tetravalent groups. For example, methyl includes monovalent methyl (—CH₃), divalent methyl (—CH₂—, methylyl), trivalent methyl

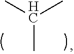

and tetravalent methyl

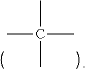

Unless otherwise specified, all numbers expressing quantities of ingredients, reaction conditions, and other properties or parameters used in the specification and claims are to be understood as being modified in all instances by the term “about.” Accordingly, unless otherwise indicated, it should be understood that the numerical parameters set forth in the following specification and attached claims are approximations. At the very least, and not as an attempt to limit the application of the doctrine of equivalents to the scope of the claims, numerical parameters should be read in light of the number of reported significant digits and the application of ordinary rounding techniques. For example, the term “about” can encompass variations of ±10%, ±5%, ±2%, ±1%, ±0.5%, or ±0.1% of the numerical value of the number, which the term “about” modifies. In various embodiments, the term “about” encompasses variations of ±5%, ±2%, +1%, or 0.5% of the numerical value of the number. In some embodiments, the term “about” encompasses variations of ±5%, ±2%, or ±1% of the numerical value of the number. In certain embodiments, the term “about” encompasses variations of ±5% of the numerical value of the number. In certain embodiments, the term “about” encompasses variations of ±2% of the numerical value of the number. In certain embodiments, the term “about” encompasses variations of ±1% of the numerical value of the number.

All numerical ranges herein include all numerical values and ranges of all numerical values within the recited range of numerical values. As a non-limiting example, (C₁-C₆) alkyls also include any one of C₁, C₂, C₃, C₄, C₅, C₆, (C₁-C₂), (C₁-C₃), (C₁-C₄), (C₁-C₅), (C₂-C₃), (C₂-C₄), (C₂-C₅), (C₂-C₆), (C₃-C₄), (C₃-C₅), (C₃-C₆), (C₄-C₅), (C₄-C₆), and (C₅-C₆) alkyls.

Further, while the numerical ranges and parameters setting forth the broad scope of the disclosure are approximations as discussed above, the numerical values set forth in the Examples section are reported as precisely as possible. It should be understood, however, that such numerical values inherently contain certain errors resulting from the measurement equipment and/or measurement technique.

### List of Abbreviations and Terms

1H-NMR: Proton Nuclear Magnetic Resonance spectroscopy

### ADME: Absorption, Distribution, Metabolism, and Excretion

AE: Adverse event
AUC₀₋₂₄: area under the concentration-time curve from time 0 to 24 hours postdose
BBB: blood-brain barrier
Cmax: maximum plasma concentration
cGMP: cyclic guanosine monophosphate
DMSO: dimethyl sulfoxide
DSFC: dorsal skin-fold chambers
F cells: blood cells with fetal haemoglobin
FIH: first in human
FTIR: Fourier transform infrared spectroscopy
GC: gas chromatography
HBB: hemoglobin subunit beta
HbF: fetal hemoglobin
HBG: gamma-globin gene
HbS: sickle hemoglobin
hERG: human ether-à-go-go related gene
HPLC: high-performance liquid chromatography
HU: hydroxyurea
IC: inhibitory concentration
IC50: a half minimal inhibitory concentration
ICAM-1: intercellular adhesion molecule-1

### ICH: International Conference on Harmonisation

ICP-MS: inductively coupled plasma mass spectroscopy
IV: intravenous
MAD: multiple-ascending dose
MTD: maximum tolerated dose
NO: nitric oxide
NOAEL: no-observed-adverse-effect level
PD: pharmacodynamic
PDE9: phosphodiester-9
PEG polyethylene glycol
PIC: Powder in capsule
PK: pharmacokinetic(s)
PKG: protein kinase G
RBC: red blood cell
RH: relative humidity
SCD: sickle cell disease
SD: standard deviation
SEM: standard error of the mean
sGC: soluble guanylyl cyclase
t½: half-life

### TK: Toxicokinetic

Tmax: time of maximum concentration
VOC: vaso-occlusive crisis
WBC: white blood cell
w/w %: weight/weight percent

### EXAMPLES

It will be appreciated that the following examples are intended to illustrate but not to limit the present invention. Various other examples and modifications of the foregoing description and examples will be apparent to a person skilled in the art after reading the disclosure without departing from the spirit and scope of the invention, and it is intended that all such examples or modifications be included within the scope of the appended claims. All publications and patents referenced herein are hereby incorporated by reference in their entirety.

### Example 1. Synthesis of PDE9 Inhibitors

The compounds of the present invention may be prepared with methods disclosed in WO 2013/053690 and/or WO 2013/110768. Compounds P1, P2, P3 and P4 may be synthesized as described below.

### Overview Schemes:

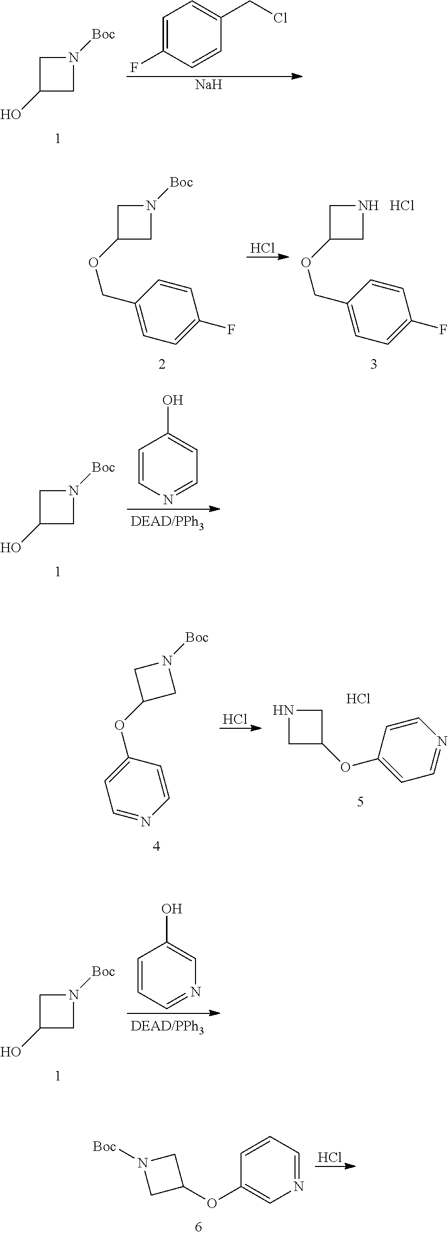

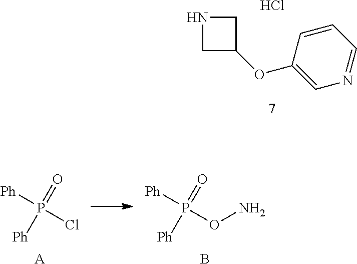

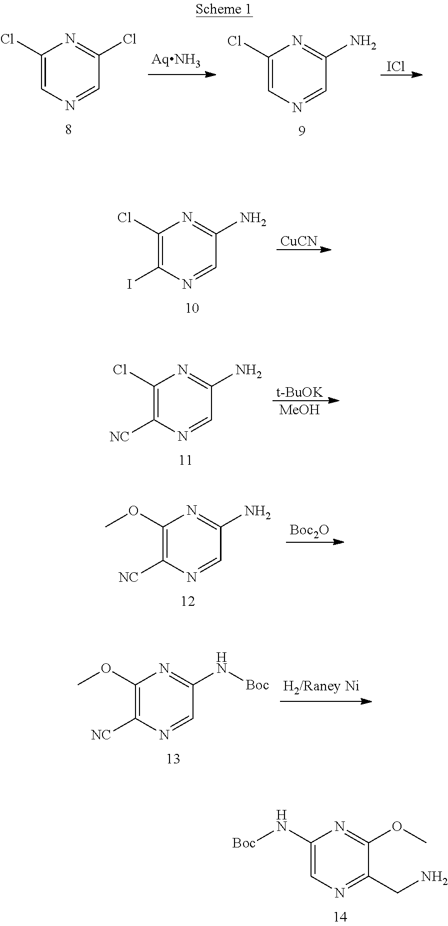

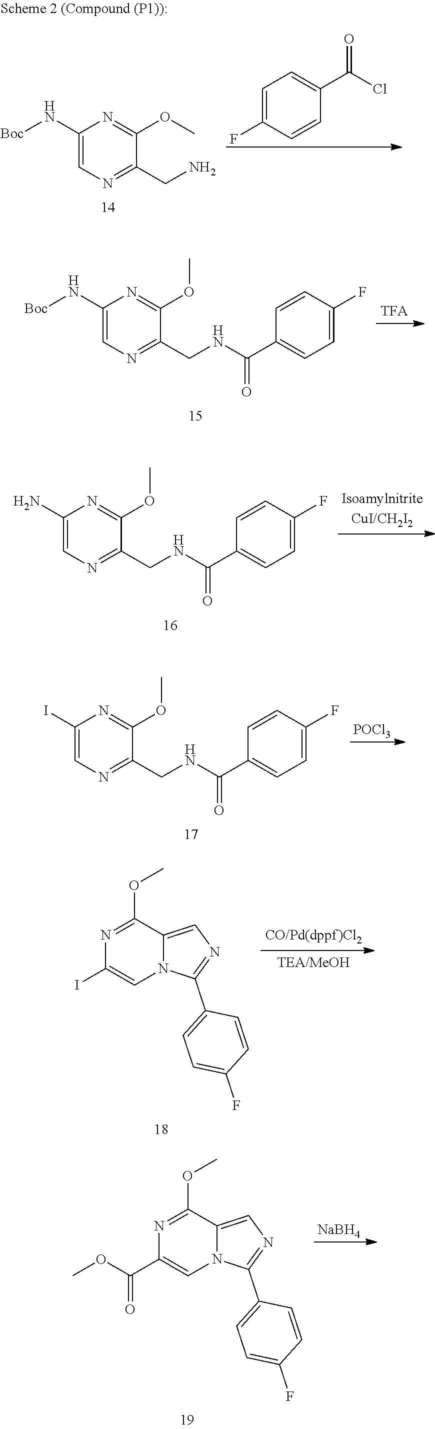

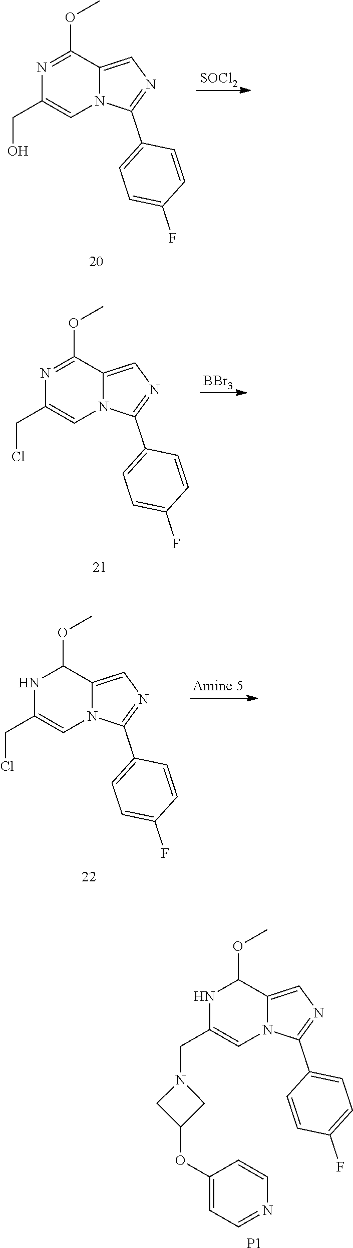

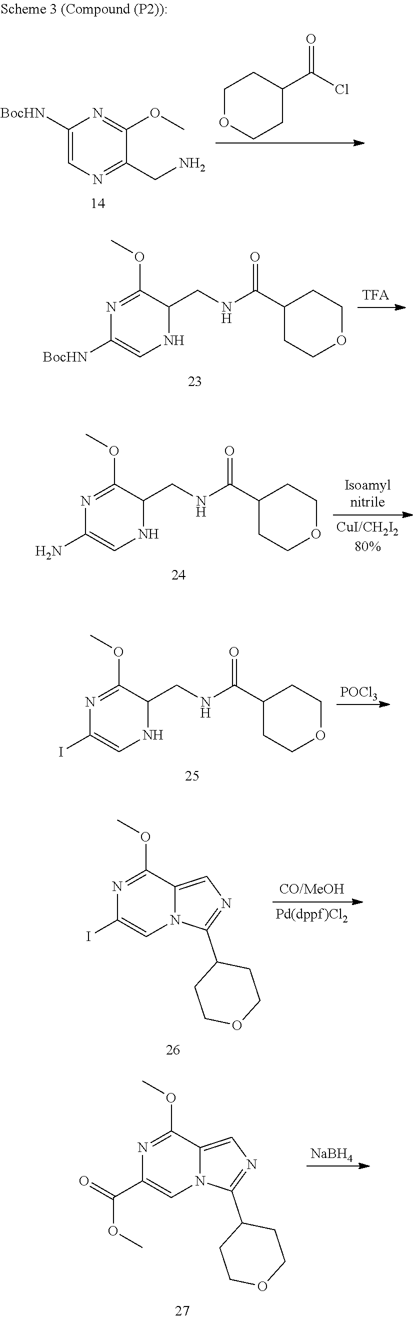

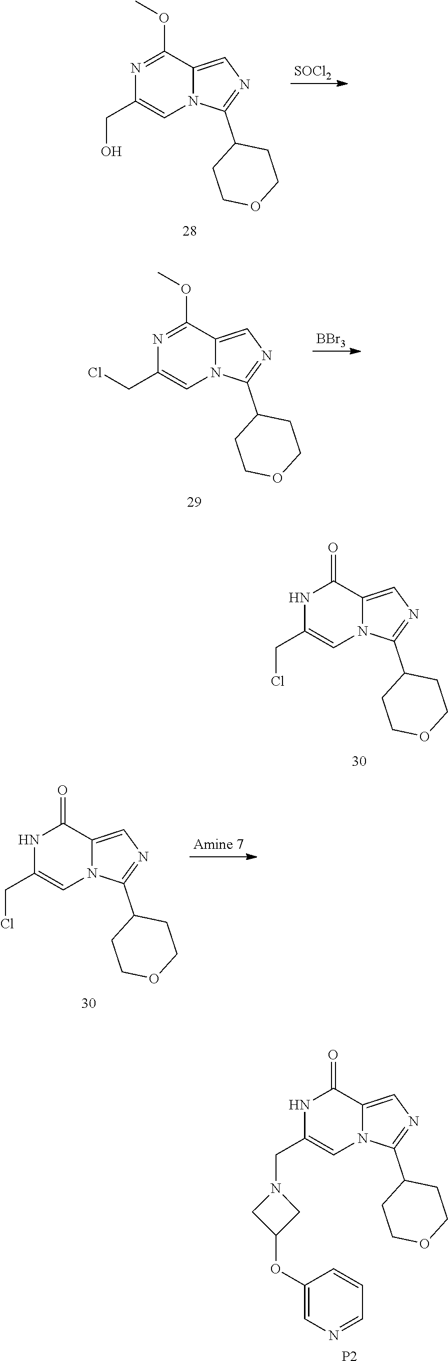

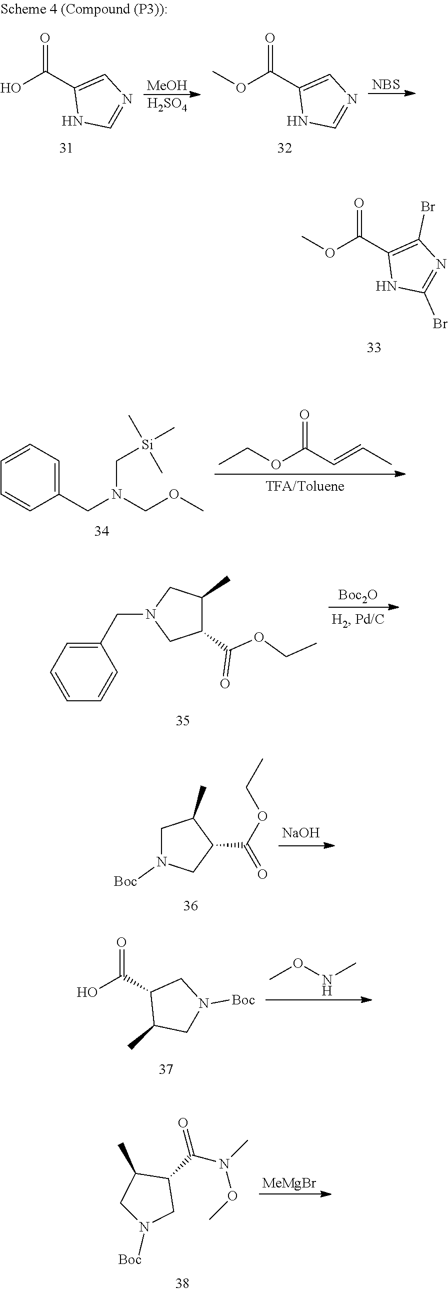

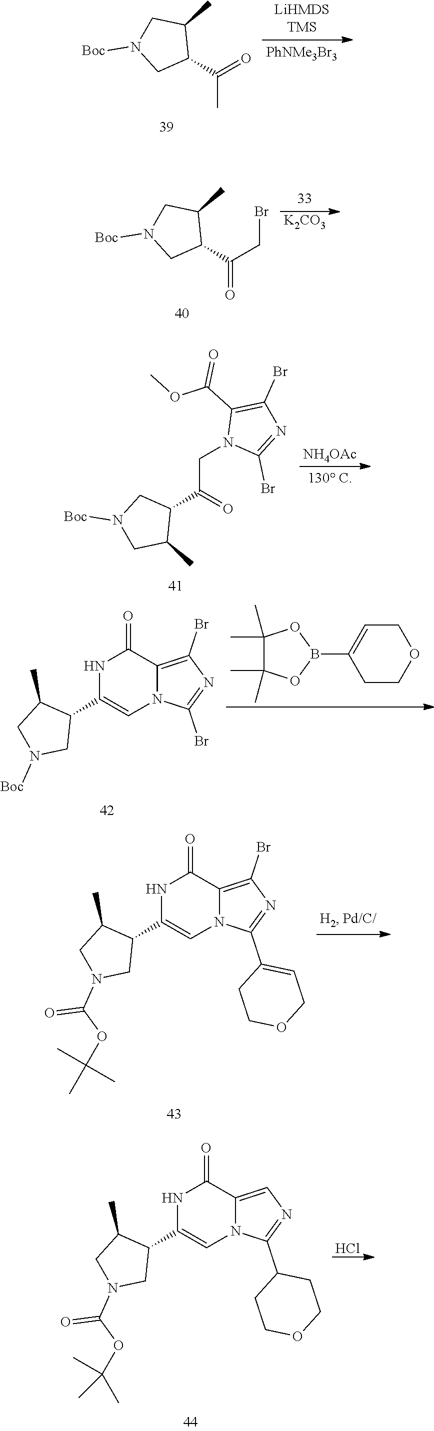

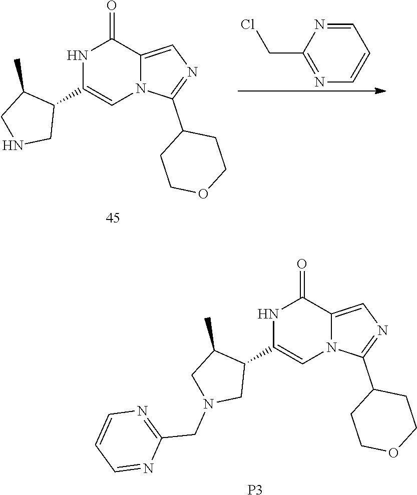 [SMILES: C[C@@H]1CNC[C@H]1c1cn2c(C3CCOCC3)ncc2c(=O)[nH]1 ; ClCc1ncccn1 ; C[C@@H]1CN(Cc2ncccn2)C[C@H]1c1cn2c(C3CCOCC3)ncc2c(=O)[nH]1 ; "P3"]

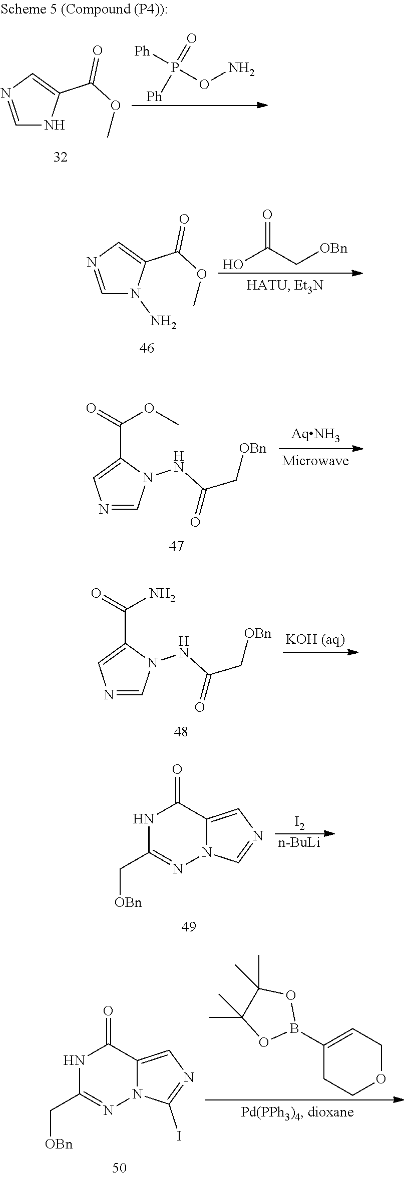

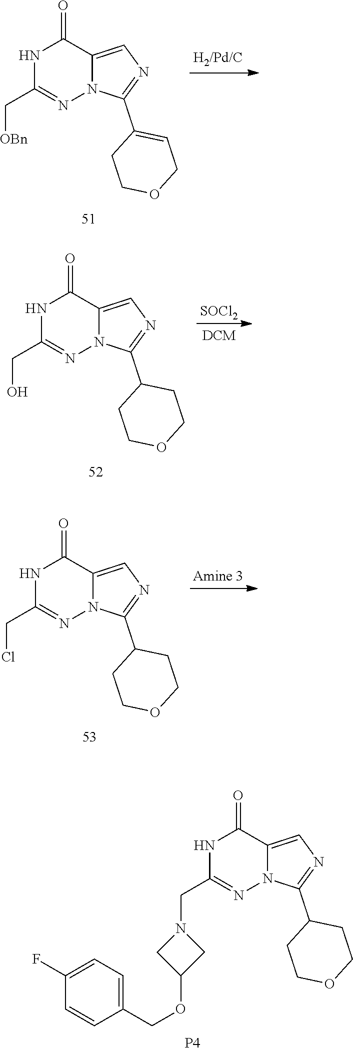

### Synthetic Procedures

### List of Abbreviations

aq aqueous

### NBS N-bromosuccinimide

Boc tert-Butoxycarbonyl
° C. degrees Celsius
CDI N,N-carbonyl dimidazole
δ_{H} chemical shift in parts per million downfield from tetramethylsilane
DCM dichloromethane
DEAD diethyl azodicarboxylate
Dppf bis(diphenylphosphino)ferrocene

### DIPEA N,N-diisopropylethylamine

### DMF N,N-dimethylformamide

eq equivalent
ESI electrospray ionization
Et ethyl
EtOAc ethyl acetate
g gram(s)
HPLC high-performance liquid chromatography
h hours
Hz hertz
J coupling constant (in NMR spectrometry)
LCMS liquid chromatography mass spectrometry
LiHMDS Lithium bis(trimethylsilyl)amide
μ micro
m multiplet (spectral); meter(s); milli
M⁺ parent molecular ion
Me methyl
MeCN acetonitrile
MeOH methanol
MHz megahertz
min minute(s)
mL milliliter
MS mass spectrometry
MTBE Methyl-tert-butyl ether
N normal (equivalents per liter)
NaOH sodium hydroxide

### NBS N-Bromosuccinimide

nm nanometer(s)
NMR nuclear magnetic resonance
PE petroleum ether bp: 60˜90° C.
RT room temperature
s singlet (spectral)
t triplet (spectral)
T temperature
TEA triethylamine
TFA trifluoroacetic acid
THE tetrahydrofuran
TLC thin layer chromatography
TMS tetramethylsilane
TMS-Cl trimethylsilyl chloride
Tol toluene

### General Experimental Methods

¹H NMR spectra were recorded on Bruker Avance III 400 MHz and Bruker Fourier 300 MHz and TMS was used as an internal standard.

LCMS was taken on a quadrupole Mass Spectrometer on Agilent LC/MSD 1200 Series (Column: ODS 2000 (50×4.6 mm, 5 μm) operating in ES (+) or (−) ionization mode; T=30° C.; flow rate=1.5 mL/min; detected wavelength: 214 nm.

### Synthesis of 6-Chloro-pyrazin-2-ylamine (9)

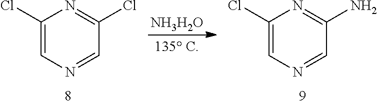

A solution of compound 8 (450.0 g, 3.02 mol) in conc. aq. NH₃ (3.0 L) was stirred at 135° C. overnight in a 10 L sealed pressure vessel. TLC and LC/MS showed complete conversion of the starting material. The reaction mixture was cooled to room temperature and filtered to afford a white solid. The solid was washed with water (200 mL×3), and then dried to afford compound 9 (312 g, 80% yield) as a solid.

¹HNMR (400 MHz, DMSO-d6): δ 7.82 (s, 1H), 7.12 (s, 1H), 6.93 (s, 2H). MS Caled.: 129 MS Found: 130 ([M+H]⁺).

### Synthesis of 6-Chloro-5-iodo-pyrazin-2-ylamine (10)

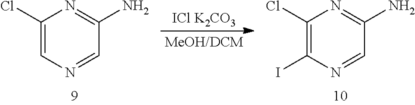 [SMILES: Nc1cncc(Cl)n1 ; "ICl K2CO3" ; Nc1cnc(I)c(Cl)n1]

To a mixture of compound 9 (312.0 g, 2.4 mol) and K₂CO₃ (664.0 g, 4.8 mol) in MeOH (1.0 L) was dropwise added ICl (704.0 g, 4.3 mol in 1.0 L of DCM) over 2 hours at 0° C. Then the reaction mixture was stirred at room temperature overnight. The reaction was quenched with Na₂SO₃ aqueous solution (2M, 1.5 L). The mixture was extracted with DCM (1.0 L×3). The combined organic phases were dried over anhydrous Na₂SO₄, filtered and concentrated. The crude product was purified by column chromatography on silica gel (PE/EA=10/1 to 4/1) to afford compound 10 (460 g, 75% yield) as a solid.

¹HNMR (400 MHz, DMSO-d6): δ 7.68 (s, 1H), 7.07 (s, 2H). MS Calcd.: 255 MS Found: 256 ([M+H]⁺).

### Synthesis of 5-Amino-3-chloro-pyrazine-2-carbonitrile (11)

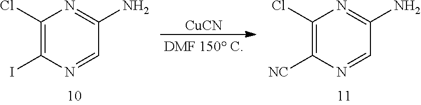

A mixture of compound 10 (460.0 g, 1.8 mol) and CuCN (177.0 g, 1.98 mol) in DMF (2.0 L) was stirred on an oil bath at 150° C. for 2 hours. LC/MS showed full conversion of the starting martial. The reaction mixture was cooled to room temperature and poured into EtOAc (1.5 L). To the resulting mixture was slowly added conc. aq. NH₃ (1.0 L), and it was then extracted with EtOAc (1.0 L×2). The combined organic phases were washed with H₂O (1.5 L×5) and brine (1.5 L) and dried over anhydrous Na₂SO₄. The organic phase was filtered and concentrated to afford compound 11 (232 g, 84% yield) as solid.

¹HNMR (400 MHz, DMSO-d6): δ 8.12 (s, 2H), 7.88 (s, 1H). MS Calcd.: 154; MS Found: 155 ([M+H]⁺).

### Synthesis of 5-Amino-3-methoxy-pyrazine-2-carbonitrile (12)

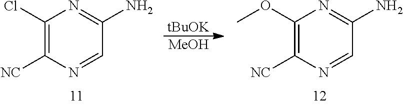

Potassium tert-butoxide (168.0 g, 1.5 mol) was added in portions into methanol (1.5 L) in a round-bottom flask. The suspension was refluxed for one hour. Then compound 11 (232.0 g, 1.5 mol) was added under an N₂ atmosphere. The resulting suspension was refluxed for 1.5 hours. After cooling to room temperature the reaction mixture was concentrated in vacuum and diluted with water (2.0 L), then extracted with EtOAc (2.0 L×5). The combined organic phases were dried with Na₂SO₄, filtered and concentrated to afford 12 (170 g, 75% yield) as a solid.

¹HNMR (300 MHz, DMSO-d6): δ 7.69 (s, 2H), 7.51 (s, 1H), 3.89 (s, 3H). MS Calcd.: 150; MS Found: 151 ([M+H]⁺).

### Synthesis of (5-Cyano-6-methoxy-pyrazin-2-yl)-carbamic acid tert-butyl ester (13)

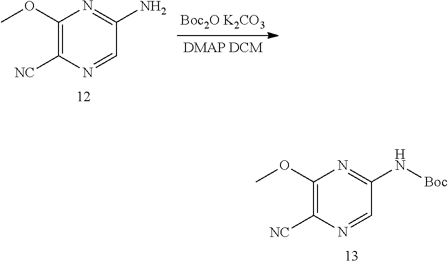 [SMILES: COc1nc(N)cnc1C#N ; COc1nc(NC(=O)OC(C)(C)C)cnc1C#N]

4-Dimethylaminopyridine (1.0 g, 0.01 mol) was added into a mixture of compound 12 (120.0 g, 0.8 mol) in DCM (1.5 L) at room temperature. Then di-tert-butyl dicarbonate (327 g, 1.5 mol) in DCM (1.0 L) was added dropwise at 10-20° C. for 2 hours. Then the reaction was stirred at room temperature overnight. The suspension dissolved and the reaction solution was diluted with 2 L of water. The DCM phase was separated and dried with sodium sulfate, filtered and concentrated in vacuum. The residue was purified by column chromatography on silica gel (PE/EtOAc=10:1) to afford 13 (150 g, 75% yield).

¹HNMR (300 MHz, DMSO-d6): δ 10.78 (s, 1H), 8.70 (s, 1H), 3.97 (s, 3H), 1.49 (s, 9H). MS Caled.: 250; MS Found: 251 ([M+H]⁺).

### Synthesis of (5-Aminomethyl-6-methoxy-pyrazin-2-yl)-carbamic acid tert-butyl ester (14)

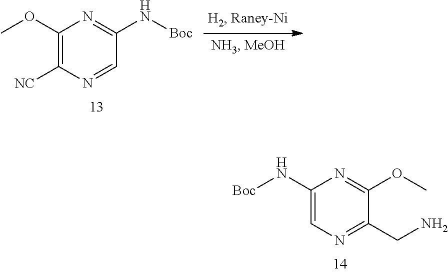 [SMILES: COc1nc(NC(=O)OC(C)(C)C)cnc1C#N ; COc1nc(NC(=O)OC(C)(C)C)cnc1CN]

Raney Ni (10.0 g) was added into a mixture of compound 13 (30.0 g, 120 mmol) in concentrated NH₃ in MeOH (500 mL) at room temperature. The suspension was stirred at room temperature under 1 atm H₂ overnight. The reaction mixture was diluted with a mixture of DCM/MeOH (1:1). The reaction mixture was filtered and the filtrate was concentrated in vacuum. The residue was triturated with PE/EtOAc=2/1 to afford 14 (23 g, 75% yield) as a solid.

¹HNMR (300 MHz, DMSO-d6): δ 8.46 (s, 1H), 3.87 (s, 3H), 3.70 (s, 2H), 3.17 (s, 3H), 1.47 (s, 9H). MS Calcd.: 254; MS Found: 255 ([M+H]⁺).

### Synthesis of 5-[(4-Fluoro-benzoylamino)-methyl]-6-methoxy-pyrazin-2-yl-carbamic acid tert-butyl ester (15)

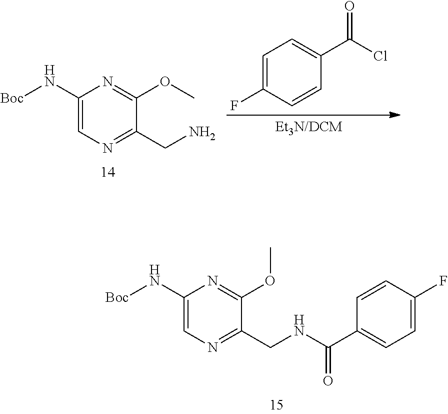

To a solution of compound 14 (4.52 g, 17.86 mmol) in DCM (200 mL) was added TEA (5.41 g, 58.53 mmol), and then 4-fluorobenzoyl chloride (3.4 g, 21.42 mmol) was added dropwise. The resulting reaction mixture was stirred at room temperature for 2 hours. TLC detected the reaction was complete. The reaction was quenched with water (100 mL). The organic phase was separated and the aqueous phase was extracted with DCM (200 mL×2). The combined organic phases were dried over anhydrous MgSO₄, filtered and concentrated in vacuum. The residue was purified by column chromatography on silica gel to afford 15 (5.77 g, 85.9% yield) as a solid.

¹HNMR (400 MHz, DMSO-d6): δ 9.89 (s, 1H), 8.81 (t, J=5.6 Hz, 1H), 8.46 (s, 1H), 7.94 (m, 2H), 7.29 (m, 2H), 4.49 (d, J=5.6 Hz, 2H), 3.90 (s, 3H), 1.47 (s, 9H). MS Calcd.: 376; MS Found: 377 ([M+H]⁺).

### Synthesis of N-(5-Amino-3-methoxy-pyrazin-2-ylmethyl)-4-fluoro-benzamide (16)

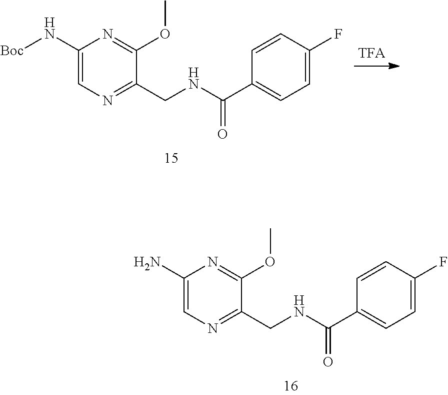 [SMILES: COc1nc(NC(=O)OC(C)(C)C)cnc1CNC(=O)c1ccc(F)cc1 ; COc1nc(N)cnc1CNC(=O)c1ccc(F)cc1]

Compound 15 (5.77 g, 15.33 mmol) was dissolved in DCM (25 mL). TFA (25 mL) was added. The reaction was stirred at room temperature overnight. TLC detected the reaction was complete. The solvent was removed. The residue was diluted with DCM (100 mL) and saturated NaHCO₃ aqueous solution (100 mL). The organic phase was separated and the aqueous phase was extracted with DCM (100 mL×2). The combined organic phases were dried over anhydrous MgSO₄, filtered and concentrated in vacuum. The residue was purified by column chromatography on silica gel (eluted with PE/EtOAc=6:1 to 1:1) to afford 16 (3.9 g, 92.2% yield) as a solid.

¹HNMR (300 MHz, CDCl₃): δ 7.90-7.85 (m, 2H), 7.46 (s, 1H), 7.40 (t, J=6.0 Hz, 1H), 7.11 (m, 2H), 4.60 (d, J=6.0 Hz, 2H), 4.37 (s, 2H), 3.93 (s, 3H). MS Calcd.: 276; MS Found: 277 ([M+H]⁺).

### Synthesis of 4-Fluoro-N-(5-iodo-3-methoxy-pyrazin-2-ylmethyl)-benzamide (17)

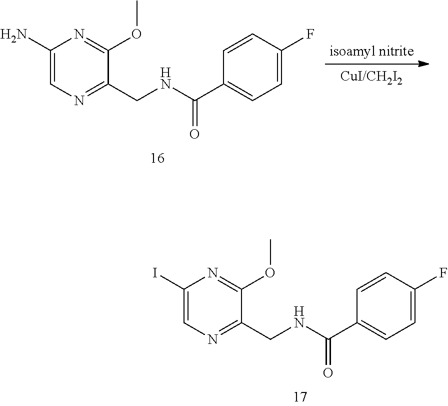 [SMILES: COc1nc(N)cnc1CNC(=O)c1ccc(F)cc1 ; COc1nc(I)cnc1CNC(=O)c1ccc(F)cc1]

Compound 16 (3.9 g, 14.1 mmol) was dissolved in anhydrous THE (100 mL). CuI (2.7 g, 14.1 mmol), then isoamyl nitrite (4.9 g, 42.3 mmol) and CH₂12 (3.8 g, 14.1 mmol) were added under N₂ gas atmosphere. The reaction mixture was heated at 75° C. for 3 hours. Then the reaction was cooled to room temperature and filtered. The filtrate was concentrated in vacuum. The residue was purified by column chromatography on silica gel (eluted with PE/EtOAc 5:1) to afford 17 (2.0 g, 37% yield) as a solid.

¹HNMR (400 MHz, CDCl₃): δ 8.34 (s, 1H), 7.88 (m, 2H), 7.36 (t, J=4.4 Hz, 1H), 7.14 (m, 2H), 4.66 (d, J=4.4 Hz, 2H), 4.04 (s, 3H). MS Calcd.: 387; MS Found: 388 ([M+H]⁺).

### Synthesis of 3-(4-Fluoro-phenyl)-6-iodo-8-methoxy-imidazo[1,5-a]pyrazine (18)

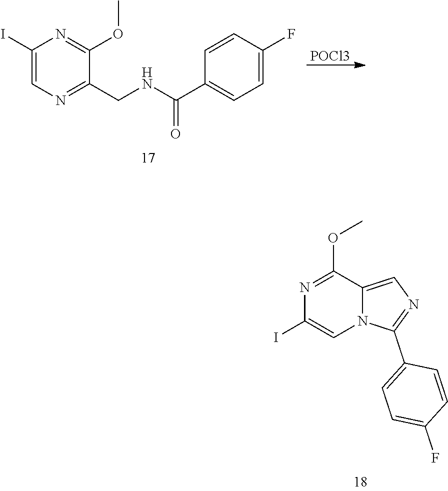

Compound 17 (1.6 g, 4.13 mmol) was suspended in MeCNCH₃CN (50 mL). POCl₃ (6.3 g, 41.3 mmol) and TEA (1.25 g, 12.39 mmol) was added under N₂ gas atmosphere and the reaction mixture was heated at 85° C. for 6 hours. The solvent was removed under reduced pressure. The residue was diluted with DCM (100 mL) and ice water (30 mL). Then saturated Na₂CO₃ aqueous solution (100 mL) was added. The organic phase was separated and the aqueous phase was extracted with DCM (100 mL×2). The combined organic phases were dried, filtered and concentrated in vacuum. The residue was purified by column chromatography on silica gel (eluted with PE/EtOAc=20:1 to 3:1) to afford 18 (1.5 g, 97.8% yield) as a solid.

¹HNMR (300 MHz, CDCl₃): δ 8.01 (s, 1H), 7.82 (s, 1H), 7.77-7.72 (m, 2H), 7.28-7.23 (m, 2H), 4.11 (s, 3H). MS Calcd.: 369; MS Found: 370 ([M+H]⁺).

### Synthesis of 3-(4-Fluoro-phenyl)-8-methoxy-imidazo[1,5-a]pyrazine-6-carboxylic acid methyl ester (19)

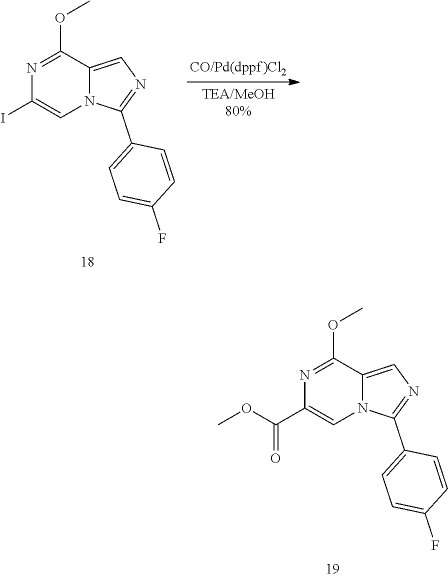 [SMILES: COc1nc(I)cn2c(-c3ccc(F)cc3)ncc12 ; COC(=O)c1cn2c(-c3ccc(F)cc3)ncc2c(OC)n1]

To a mixture solution of 18 (4.11 g, 11.13 mmol), CuI (640 mg, 3.34 mmol) and Pd(dppf)₂Cl₂ (930 mg, 1.11 mmol) in MeOH (100 mL) was added TEA (14 mL). The reaction mixture was heated to 85° C. under a CO atmosphere (3.0 MPa) for 16 hours. The reaction mixture was allowed to cool to room temperature and concentrated in vacuo to get the crude product. The residue was purified by column chromatography on silica gel (eluted with PE/EtOAc=1:1) to afford 19 (2.3 g, 75% yield) as a solid.

¹H NMR (400 MHz, CDCl₃): δ 8.59 (s, 1H), 7.87 (s, 1H), 7.78 (m, 2H), 7.28 (m, 2H), 4.21 (s, 3H), 3.96 (s, 3H). MS Calcd.: 301; MS Found: 302 ([M+H]⁺).

### Synthesis of [3-(4-Fluoro-phenyl)-8-methoxy-imidazo[1,5-a]pyrazin-6-yl]-methanol (20)

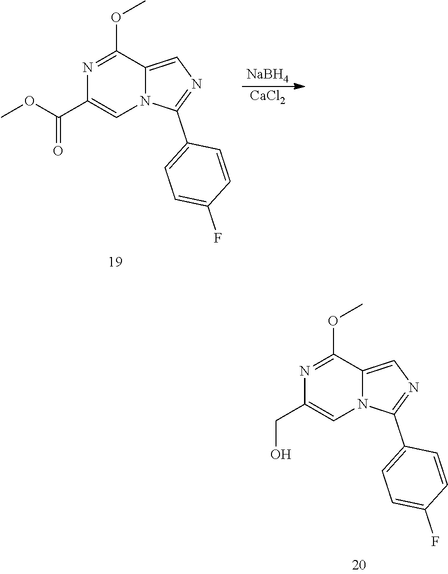

A mixture of powered anhydrous CaCl₂) (4.23 g, 38.15 mmol) and NaBH₄ (2.86 g, 76.3 mmol) in THE (100 mL) was stirred at room temperature for 1 hour. A solution of compound 19 (2.3 g, 7.63 mmol) in THE (25 mL) was added and then MeOH (25 mL) was added. The reaction mixture was stirred at room temperature for 1.5 hours. The mixture reaction was quenched with water (50 mL). After removing the organic solvent under reduced pressure, the resulting solution was dissolved in EtOAc (200 mL) and water (50 mL). The separated aqueous phase was extracted with EtOAc (3×100 mL). Then the combined organic phases were concentrated under reduced pressure. The residue was purified by column chromatography on silica gel (eluted with PE/EtOAc=2:1) to afford the desired product compound 20 (1.93, 93% yield) as a solid.

¹H NMR (400 MHz, CDCl₃): δ 7.81 (s, 1H), 7.79-7.74 (m, 3H), 7.25-7.22 (m, 2H), 4.56 (d, J=4.4 Hz, 2H), 4.11 (s, 3H), 2.41 (t, J=4.4 Hz, 1H). MS Calcd.: 273; MS Found: 274 ([M+H]⁺).

### Synthesis of 6-Chloromethyl-3-(4-fluoro-phenyl)-8-methoxy-imidazo[1,5-a]pyrazine (21)

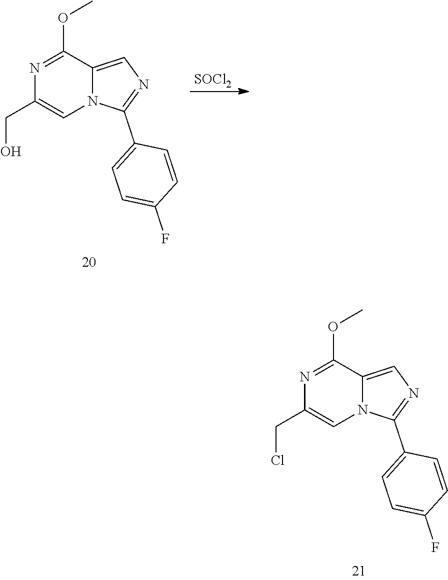

To a solution of 20 (1.88 g, 6.88 mmol) in dichloromethane (100 mL) was added dropwise thionyl chloride (4.5 mL) while cooling on an ice-water bath. After the addition, the mixture was stirred for another 2 hours. The reaction mixture was quenched with ice water, washed with brine (20 mL), dried over Na₂SO₄ and concentrated in vacuo to afford 21 (2.01 g, 100% yield) as a solid.

¹H NMR (400 MHz, CDCl₃): δ 7.87 (s, 1H), 7.83-7.79 (m, 3H), 7.30-7.27 (m, 2H), 4.50 (s, 2H), 4.12 (s, 3H). MS Calcd.: 291; MS Found: 292 ([M+H]⁺).

### Synthesis of 6-Chloromethyl-3-(4-fluoro-phenyl)-7H-imidazo[1,5-a]pyrazin-8-one (22)

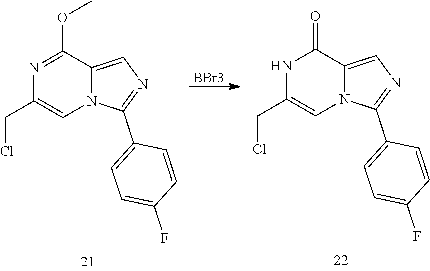

To a solution of 21 (1.87 g, 6.41 mmol) in MeOH (50 mL) was added 6N aqueous HCl and the resulting solution was stirred at 70° C. for one hour. The mixture was concentrated to afford the product 22 (1.60 g, 90% yield) as a white solid.

¹H NMR (300 MHz, DMSO-d6): δ 11.29 (s, 1H), 8.07 (s, 1H), 7.83-7.87 (m, 2H), 7.74 (s, 1H), 7.46-7.50 (m, 2H), 4.59 (s, 2H). MS Caled.: 277; MS Found: 278 ([M+H]⁺).

### Synthesis of 4-(Azetidin-3-yloxy)-pyridine hydrochloride salt (5)

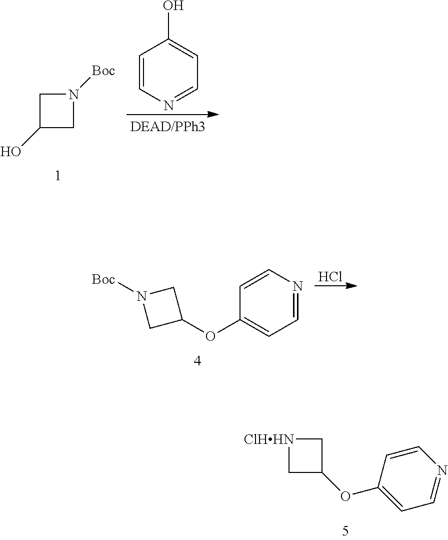

To a solution of tert-butyl 3-hydroxyazetidine-1-carboxylate 1 (4.55 g, 26.3 mmol) in THE (100 mL) was added pyridin-4-ol (2.0 g, 21.0 mmol), PPh₃ (6.89 g, 26.3 mmol) and DEAD (4.57 g, 26.3 mmol). The resulting reaction mixture was stirred at 70° C. overnight. TLC indicated that the reaction was complete. The reaction mixture was concentrated in vacuum. The resulting oil was dissolved in 1.0 M aqueous HCl solution (, 20 mL) and extracted with DCM (50 mL×3), The combined organic phases were washed with HCl (aq) solution (0.5 M, 150 mL). The aqueous fractions were combined and basified to pH12 using NaOH (1.0 M) and extracted with DCM (100 mL×3). The combined organic phases were dried over anhydrous Na₂SO₄, filtered and concentrated in vacuum. The residue was purified by column chromatography on silica gel to afford to afford 4 (2.81 g, 53% yield) as a solid.

¹HNMR (400 MHz, DMSO-d6): δ 8.41 (d, J=6.0 Hz, 2H), 6.88 (d, J=6.0 Hz, 2H), 5.07-5.09 (m, 1H), 4.32-4.33 (m, 2H), 3.80-3.82 (m, 2H), 1.39 (s, 9H). MS Calcd.: 250; MS Found: 251 ([M+H]⁺).

To a solution of 4 (2.81 g, 11.2 mmol) in Et₂O (100 mL) was added HCl in Et₂O (20 mL). The resulting reaction mixture was stirred at room temperature overnight. TLC indicated that the reaction was complete. The reaction mixture was filtered and the solid was dried to afford 5 (1.82 g, 87% yield).

¹HNMR (300 MHz, DMSO-d6): δ 9.58 (s, 2H), 8.77-8.79 (m, 2H), 7.48-7.49 (m, 2H), 5.40-5.45 (m, 1H), 4.49-4.51 (m, 2H), 4.07-4.11 (m, 2H). MS Calcd.: 150; MS Found: 151 ([M+H]⁺).

### Synthesis of 3-(4-fluorophenyl)-6-((3-(pyridin-4-yloxy)azetidin-1-yl)methyl)imidazo[1,5-a]pyrazin-8(7H)-one (P1)

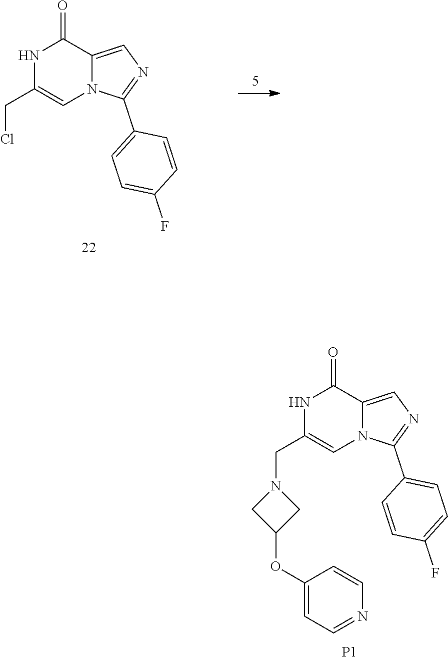

To a mixture of compound 22 (1.5 g, 5.4 mmol) and 5 (1.31 g, 7.0 mmol) in MeCN (100 mL) was added DIPEA (6.96 g, 5.4 mmol). The reaction mixture was heated and refluxed overnight. The solvent was removed in vacuum. The residue was purified by flash column chromatography on reverse phase silica gel (eluted by 5%95% MeCN in water) to afford desired product P1(1.28 g, 62% yield) as a solid.

¹H NMR (400 MHz, DMSO-d6): δ 10.7 (s, 1H), 8.37 (d, J=6.0 Hz, 2H), 7.85 (s, 1H), 7.85-7.82 (m, 2H), 7.42 (m, 2H), 7.34 (s, 1H), 6.86 (d, J=6.0 Hz, 2H), 4.93 (m, 1H), 3.88-3.77 (m, 2H), 3.42 (s, 2H), 3.18-3.14 (m, 2H). MS Calcd.: 391; MS Found: 392 ([M+H]⁺).

### Synthesis of (6-Methoxy-5-{[(tetrahydro-pyran-4-carbonyl)-amino]-methyl}-pyrazin-2-yl)-carbamic acid tert-butyl ester (23)

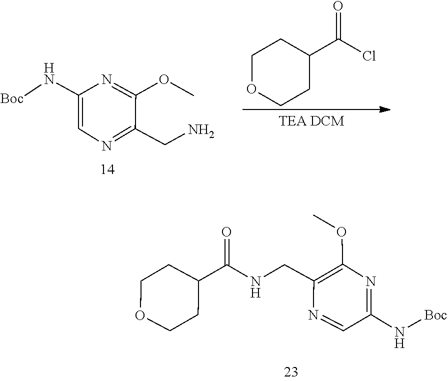 [SMILES: COc1nc(NC(=O)OC(C)(C)C)cnc1CN ; O=C(Cl)C1CCOCC1 ; COc1nc(NC(=O)OC(C)(C)C)cnc1CNC(=O)C1CCOCC1]

To a solution of compound 14 (28.4 g, 0.11 mol) in DCM (200 mL) was added TEA (49 mL, 0.34 mol), then tetrahydropyran-4-carbonyl chloride (17.5 g, 0.13 mol) was added dropwise. The resulting reaction mixture was stirred at room temperature overnight. TLC indicated that the reaction was complete. The reaction was quenched with water (100 mL). The organic phase was separated and the aqueous phase was extracted with DCM (200 mL×2). The combined organic phases were dried over anhydrous Na₂SO₄, filtered and concentrated in vacuum. The residue was purified by column chromatography on silica gel (PE/EA=5/1 to 1/3) to afford 23 (31 g, 75% yield) as a solid.

¹H NMR (DMSO-d6, 400 MHz): δ 9.89 (s, 1H), 8.47 (s, 1H), 8.10-8.07 (t, J=5.2 Hz, 1H), 4.29-4.28 (d, J=5.2 Hz, 2H), 3.87 (s, 3H), 3.85-3.82 (m, 2H), 3.32-3.25 (m, 2H), 2.45-2.43 (m, 1H), 1.60-1.55 (m, 4H), 1.48 (s, 9H). MS Calcd.: 366; MS Found: 367 ([M+H]⁺).

### Synthesis of Tetrahydro-pyran-4-carboxylic acid (5-amino-3-methoxy-pyrazin-2-ylmethyl)-amide (24)

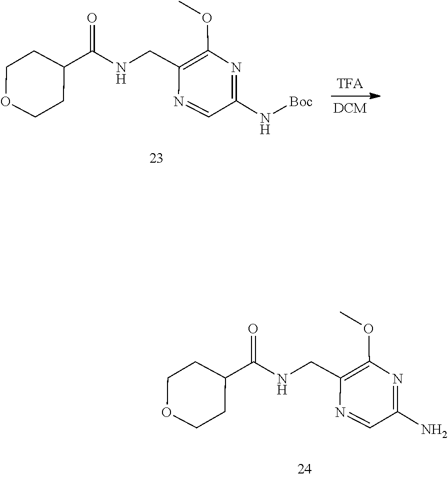 [SMILES: COc1nc(NC(=O)OC(C)(C)C)cnc1CNC(=O)C1CCOCC1 ; COc1nc(N)cnc1CNC(=O)C1CCOCC1]

Compound 23 (19.0 g, 0.08 mol) was dissolved in DCM (100 mL). TFA (100 mL) was added. The reaction was stirred at room temperature overnight. TLC indicated that the reaction was complete. The solvent was removed. The residue was diluted with DCM (100 mL) and saturated NaHCO₃ aqueous solution (100 mL). The aqueous phase was extracted with DCM (100 mL×2). The combined organic phases were dried over anhydrous Na₂SO₄, filtered and concentrated in vacuum. The residue was purified by column chromatography on silica gel (PE/EA=6/1 to 1/1) to afford 24 (19 g, 85% yield) as a solid.

¹H NMR (DMSO-d6, 400 MHz): δ 7.87 (t, J=4.8 Hz, 1H), 7.36 (s, 1H), 6.26 (br. s, 2H), 4.16 (d, J=4.8 Hz, 2H), 3.86-3.82 (m, 2H), 3.80 (s, 3H), 3.30-3.24 (m, 2H), 2.41 (m, 1H), 1.59-1.54 (m, 4H). MS Calcd.: 266; MS Found: 267 ([M+H]⁺).

### Synthesis of Tetrahydropyran-4-carboxylic acid (5-iodo-3-methoxy-pyrazin-2-ylmethyl)-amide (25)

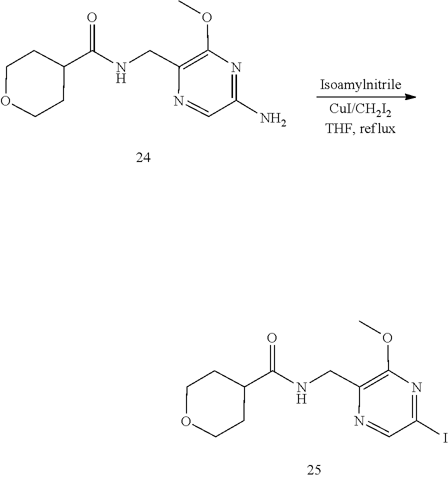 [SMILES: COc1nc(N)cnc1CNC(=O)C1CCOCC1 ; COc1nc(I)cnc1CNC(=O)C1CCOCC1]

To a mixture of compound 24 (15.5 g, 58.4 mmol), CH₂12 (23.5, 87.6 mmol) and isoamyl nitrite (23.9 g, 204 mmol) in THF (600 mL) was added CuI (11.3 g, 39.6 mmol) under an N₂ atmosphere. The reaction mixture was stirred at 80° C. for 7 hours. The precipitate was filtered. The filtrate was concentrated and purified by column chromatography (MeOH/DCM=1/20) to get crude product, then purified by flash column chromatography on reverse phase silica gel (eluted by 5%95% MeCN in water) to afford desired product compound 25 (4.5 g, 20% yield) as a solid.

¹H NMR (DMSO-d6, 300 MHz): δ 8.41 (s, 1H), 8.16 (t, J=5.4 Hz, 1H), 4.28 (d, J=5.4 Hz, 2H), 3.92 (s, 3H), 3.87-3.81 (m, 2H), 3.30-3.24 (m, 2H), 2.49 (m, 1H), 1.60-1.56 (m, 4H). MS Calcd.: 377 MS Found: 378 ([M+H]⁺).

### Synthesis of 6-Iodo-8-methoxy-3-(tetrahydro-pyran-4-yl)-imidazo[1,5-a]pyrazine (26)

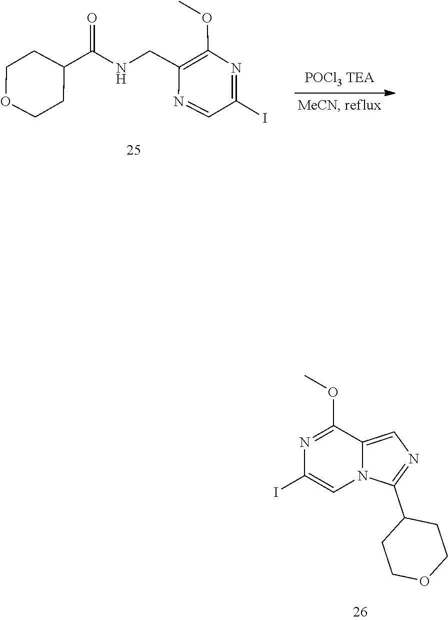 [SMILES: COc1nc(I)cnc1CNC(=O)C1CCOCC1 ; COc1nc(I)cn2c(C3CCOCC3)ncc12]

To a solution of compound 25 (4.5 g, 16.9 mmol) in MeCN (100 mL) was added POCl₃ (18 g, 118 mmol). The reaction was stirred at reflux overnight under an N₂ atmosphere. The solvent was removed under reduced pressure. The residue was treated with ice water (30 mL) and DCM (150 mL). The pH was adjusted to 7-8 by saturated Na₂CO₃ solution. The separated aqueous phase was extracted with DCM (100 mL×4). The combined organic phases were concentrated under reduced pressure to afford desired 26 (4.2 g, 99% yield) as a solid.

¹H NMR (DMSO-d6, 400 MHz): δ 8.46 (s, 1H), 7.64 (s, 1H), 3.98 (s, 3H), 3.94 (m, 2H), 3.53-3.47 (m, 3H), 1.81-1.77 (m, 4H). MS Calcd.: 359; MS Found: 360 ([M+H]⁺).

### Synthesis of 8-Methoxy-3-(tetrahydro-pyran-4-yl)-imidazo[1,5-a]pyrazine-6-carboxylic acid methyl ester (27)

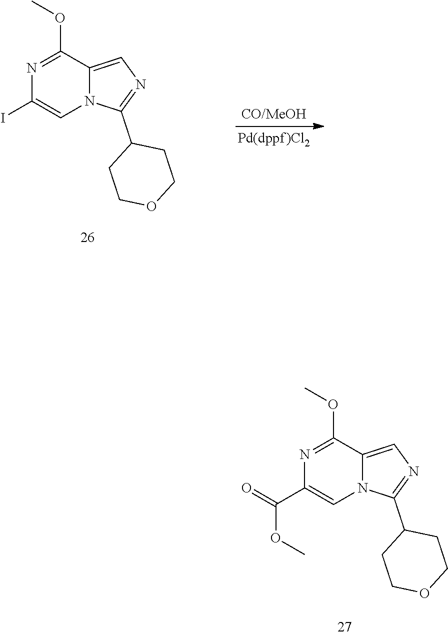 [SMILES: COc1nc(I)cn2c(C3CCOCC3)ncc12 ; COC(=O)c1cn2c(C3CCOCC3)ncc2c(OC)n1]

To a suspension of compound 26 (4.2 g, 11.7 mmol) in MeOH (100 mL) was added CuI (0.7 g, 3.0 mmol), Pd(dppf)₂Cl₂(1.0 g, 1.17 mmol) and TEA (16 mL). The reaction mixture was stirred on an oil bath set at 85° C. for 16 hours under a CO atmosphere (3 MPa). The precipitate was filtered and the filtrate was evaporated under reduced pressure. The residue was purified by column chromatography (eluted by EtOAc/PE=2/1 to MeOH/DCM=1/20) to afford desired 27 (2.7 g, 80% yield) as a solid.

¹H NMR (CDC₃, 400 MHz): δ 8.32 (s, 1H), 7.70 (s, 1H), 4.17 (s, 3H), 4.14 (m, 2H), 3.98 (s, 3H), 3.66-3.60 (m, 2H), 3.31-3.26 (m, 1H), 2.17-2.13 (m, 2H), 1.93 (m, 2H). MS Calcd.: 291; MS Found: 292 ([M+H]⁺).

### Synthesis of[8-Methoxy-3-(tetrahydro-pyran-4-yl)-imidazo[1,5-a]pyrazin-6-yl]-methanol (28)

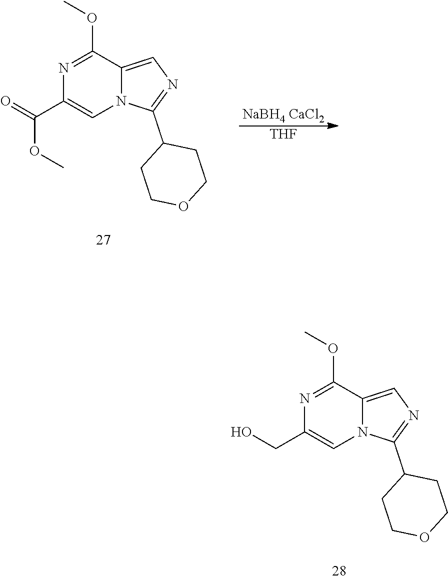 [SMILES: COC(=O)c1cn2c(C3CCOCC3)ncc2c(OC)n1 ; "NaBH4 CaCl2" ; COc1nc(CO)cn2c(C3CCOCC3)ncc12 ; "THF"]

A mixture of powered anhydrous CaCl₂)(2.4 g, 21.5 mmol) and NaBH₄ (1.6 g, 42.9 mmol) was stirred in THE (100 mL) for 1 hour at RT. A solution of compound 27 (2.4 g, 4.29 mmol) in THF (25 mL) was added and then MeOH (25 mL) was added. The reaction mixture was stirred at room temperature for 1.5 hours. The mixture reaction was quenched with water (50 mL). After removing the organic solvent under reduced pressure, the residue was partitioned between EtOAc (200 mL) and water (50 mL). The separated aqueous phase was extracted with EtOAc (100×3 mL). Then the combined organic phases were concentrated under reduced pressure. The residue was purified by column chromatography on silica gel (eluted by DCM/MeOH=100/1 to 30/1) to afford the desired product compound 28 as a solid (1.87, 80% yield).

¹H NMR (CDCl₃, 400 MHz): δ 7.65 (s, 1H), 7.43 (s, 1H), 4.58 (s, 2H), 4.13 (d, J=12.0 Hz, 2H), 4.07 (s, 3H), 3.60 (dd, J=10.4 Hz, 10.8 Hz, 2H), 3.24-3.17 (m, 1H), 2.60 (m, 1H), 2.18-2.06 (m, 2H), 1.90 (d, J=12.8 Hz, 2H). MS Calcd.: 263; MS Found: 264 ([M+H]⁺).

### Synthesis of 6-Chloromethyl-3-(tetrahydropyran-4-yl)-7H-imidazo[1,5-a]pyrazin-8-one (30)

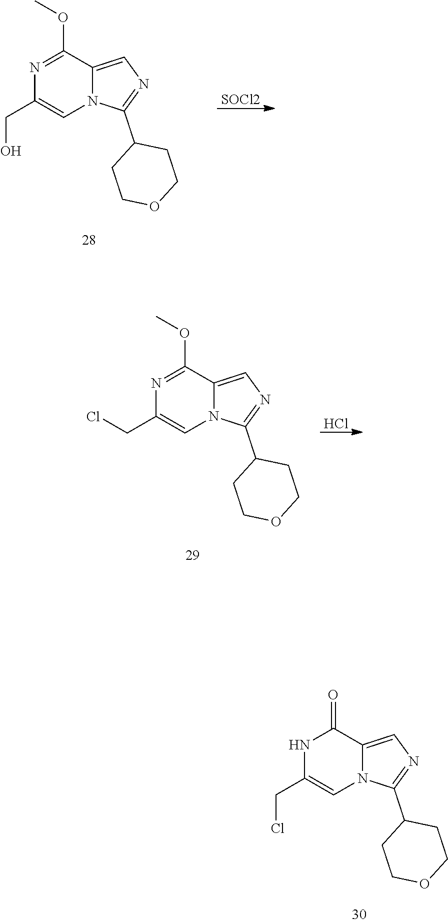 [SMILES: COc1nc(CO)cn2c(C3CCOCC3)ncc12 ; "SOCl2" ; COc1nc(CCl)cn2c(C3CCOCC3)ncc12 ; Cl ; O=c1[nH]c(CCl)cn2c(C3CCOCC3)ncc12]

To a solution of compound 28 (1.9 g, 7.11 mmol) in DCM (100 mL) was added SOCl₂(5 mL) at 0° C., then the reaction mixture was stirred at room temperature for 5 hours. TLC and LC-MS showed that the starting martial had been consumed. Then the mixture solution was concentrated and the residue was dissolved in HCl (aq.) solution (6N, 20 mL). The mixture reaction was stirred at room temperature for 10 minutes. The reaction mixture was then concentrated under reduced pressure to afford the desired product compound 29 (1.90 g, 95% yield) as a solid.

¹H NMR (DMSO-d6, 300 MHz): δ 11.49 (s, 1H), 8.28 (s, 1H), 8.00 (s, 1H), 4.55 (s, 2H), 3.97 (dd, J=2.4 Hz, 2.8 Hz, 2H), 3.53-3.43 (m, 3H), 1.95-1.81 (m, 4H). MS Calcd.: 267 MS Found: 268 ([M+H]⁺).

### Synthesis of 3-(azetidin-3-yloxy)-pyridine hydrochloride (7)

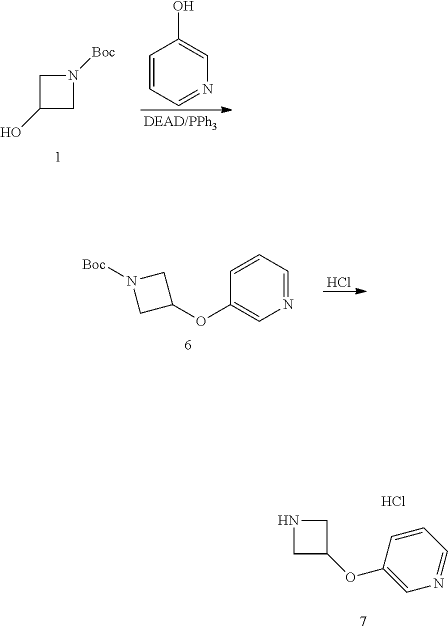 [SMILES: CC(C)(C)OC(=O)N1CC(O)C1 ; Oc1cccnc1 ; CC(C)(C)OC(=O)N1CC(Oc2cccnc2)C1 ; Cl ; Cl ; c1cncc(OC2CNC2)c1]

Compound 7 was prepared by a similar procedure to the one employed for the preparation of amine 5.

Analytical data for 7: ¹H NMR ((DMSO-d₆, 400 MHz): δ 9.73 (br d, 2H), 8.55 (d, J=2.4 Hz, 2H), 8.47 (d, J=4.4 Hz, 2H), 7.88-7.75 (m, 2H), 5.28 (t, J=5.6 Hz, 1H), 4.50-4.43 (m, 2H), 4.08-4.00 (m, 2H). MS Caled.: 150, MS Found: 151 ([M+H]⁺).

### Synthesis of 6-[3-(pyridin-3-yloxy)-azetidin-1-ylmethyl]-3-(tetrahydropyran-4-yl)-7H-imidazo[1,5-a]pyrazin-8-one (P2)

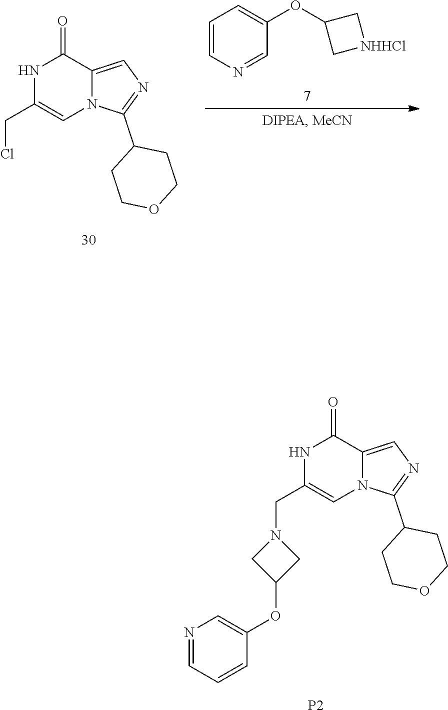

To a mixture of compound 30 (550 mg, 2.05 mmol) and 7 (500 mg, 2.67 mmol) in MeCN (200 mL) was added DIPEA (2.7 g, 20.5 mmol). The reaction mixture was refluxed overnight. The solvent was removed in vacuum. The crude product was purified by flash column chromatography on reverse phase silica gel (eluted by 5%-95% MeCN in water) to afford desired product P2 (360 mg, 46% yield) as a solid.

¹H NMR (CDC₃, 300 MHz): δ 8.26 (d, J=4.0 Hz 1H), 8.22 (s, 1H), 8.20 (d, J=2.8 Hz, 1H), 7.91 (s, 1H), 7.24-7.21 (m, 1H), 7.07 (d, J=2.8 Hz, 1H), 6.79 (s, 1H), 4.86 (m, 1H), 4.13 (m, 2H), 3.89 (t, J=7.6 Hz, 2H), 3.57 (m, 2H), 3.50 (s, 2H), 3.28 (dd, J=2.4 Hz, 6.8 Hz, 2H), 3.10-30.6 (m, 1H), 2.14-2.08 (m, 2H), 1.87 (m, 2H). MS Calcd.: 381; MS Found: 382 ([M+H]⁺).

### Synthesis of 3H-imidazole-4-carboxylic acid methyl ester (32)

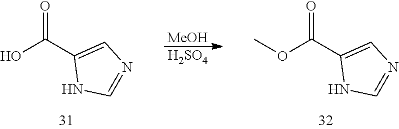

To a solution of compound 31 (25 g, 0.22 mol) in MeOH (300 mL) was added H₂SO₄ (24 mL). The mixture was stirred at reflux for 18 hours. Then pH of the reaction solution was adjusted to ˜7. The reaction mixture was concentrated in vacuo. The residue was dissolved in 100 ml of MeOH and stirred at room temperature for 15 minutes. The mixture solution was filtered and the filtrate was concentrated to afford the crude 32 (28 g, 100% yield) as a solid, which was used for next step without further purification.

¹H NMR (400 MHz, DMSO-d₆): δ 7.80 (s, 2H), 3.57 (s, 3H).

### Synthesis of 3H-imidazole-4-carboxylic acid methyl ester (33)

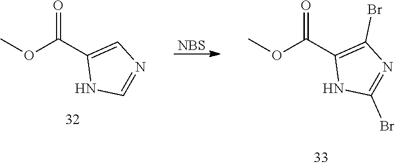

To a solution of compound 32 (22 g, 0.18 mol) in MeCN (500 mL) was added NBS (66 g, 0.37 mol). The mixture was stirred at 70° C. for 4 hours. The reaction mixture was concentrated in vacuo. The crude product was purified by column chromatography on silica gel (eluting with PE/EtOAc=5:1 to 1:1) to afford compound 33 (20 g, 40% yield) as a solid.

¹H NMR (400 MHz, DMSO-d₆): δ 14.35 (br, 1H), 3.81 (s, 3H).

### Synthesis of racemic trans-1-benzyl-4-methyl-pyrrolidine-3-carboxylic acid ethyl ester (35)

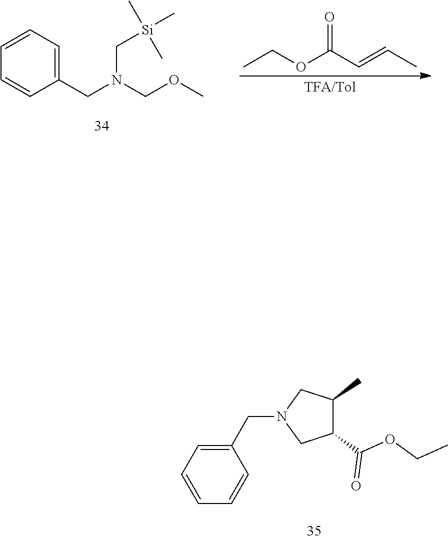

To a solution of 34 (69 g, 0.29 mol) in toluene was added but-2-enoic acid ethyl ester (50 g, 0.44 mol) and TFA (25 mL, 0.32 mol). The resulting solution was stirred at 50° C. under N₂ overnight. To the reaction mixture was added saturated aqueous NaHCO₃ solution (300 mL), and the aqueous phase was extracted with EtOAc (500 mL×3). The combined organic layers were washed with brine (300 mL), dried over Na₂SO₄, filtered and concentrated in vacuo. The crude product was purified by flash chromatography (PE/EA=20:1 to 6:1) to afford the desired racemic trans product 35 (41 g, 57% yield) as an oil.

### Synthesis of (S,S)-trans-1-benzyl-4-methyl-pyrrolidine-3-carboxylic acid ethyl ester (S,S)-(35)

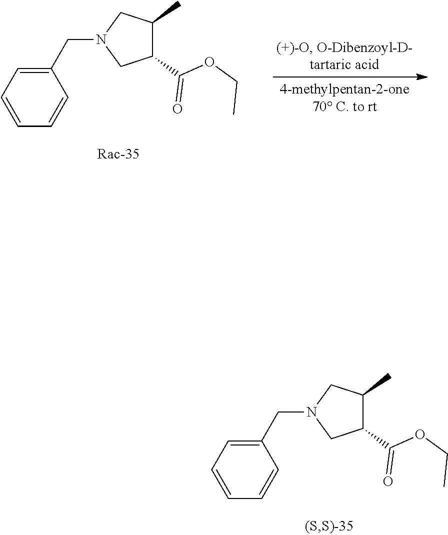 [SMILES: CCOC(=O)[C@@H]1CN(Cc2ccccc2)C[C@H]1C ; CCOC(=O)[C@@H]1CN(Cc2ccccc2)C[C@H]1C]

To a solution of Rac-35 (37 g, 0.15 mol) in 4-methyl-2-pentanone was added (−)-dibenzoyl-L-tartaric acid (34.78 g, 0.65 eq.) and the resulting reaction mixture was heated to 72° C. for 1 hr after which it was allowed to cool to RT where it was maintained for 4 hrs. The resulting solid was filtered off and the filtrate was washed with conc. aq. sodium carbonate (55 mL). The aqueous phase was extracted with 4-methyl-2-pentanone (15 mL) and the combined organic phases were washed with brine (40 mL). The organic phase was then treated with (+)-dibenzoyl-D-tartaric acid (32.16 g) and heated to 72° C. for 1 hr. The reaction mixture was cooled to RT and maintained at this temperature for 4 hrs. The solid was filtered off and dried on the filter. The solid was then recrystallized by adding a mixture of MTBE-MeOH (2:1, 270 mL), heating to 70° C. for 1 hr and allowing the product to precipitate at RT for 4 hrs. The resulting solid was filtered off, washed with MTBE and dried. Two more recrystallization following the same procedure afforded the pure product as a (+)-dibenzoyl-D-tartaric acid salt (>98% ee with based on the isolated free base).

The free base was liberated by the following procedure: the filtered solid was partitioned between MTBE (250 mL) and conc. aq. sodium carbonate (250 mL) and the aqueous phase was extracted with MTBE (125 mL). The combined organic phases were washed with water (250 mL) and brine (50 mL) and evaporated to give the product as a clear oil (13.79 g, 0.056 mol) as a clear oil.

### Synthesis of racemic trans-4-methyl-pyrrolidine-1,3-dicarboxylic acid 1-tert-butyl ester 3-methyl ester rac-(36)

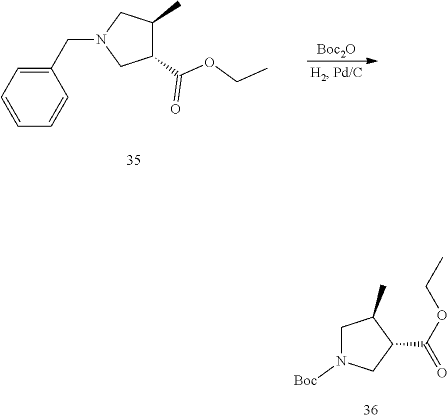 [SMILES: "Boc2O" ; CCOC(=O)[C@@H]1CN(Cc2ccccc2)C[C@H]1C ; CCOC(=O)[C@@H]1CN(C(=O)OC(C)(C)C)C[C@H]1C]

To a solution of 35 (41 g, 0.17 mol) and Boc₂O (43 g, 0.20 mol) in EtOH (500 mL) was added Pd/C (5%, 10.0 g). The reaction mixture was stirred at 50° C. for 48 hours under an atmosphere of H₂ (50 Psi). The reaction mixture was filtered and concentrated in vacuo. The crude product was purified by flash chromatography (PE/EA=20/1) to afford the desired racemic trans 36 (20 g, 46% yield) as an oil.

### Synthesis of (S,S)-trans-4-methyl-pyrrolidine-1,3-dicarboxylic acid 1-tert-butyl ester (S,S)-(37) via (S,S)-trans-4-methyl-pyrrolidine-1,3-dicarboxylic acid 1-tert-butyl ester 3-methyl ester (S,S)-(36)

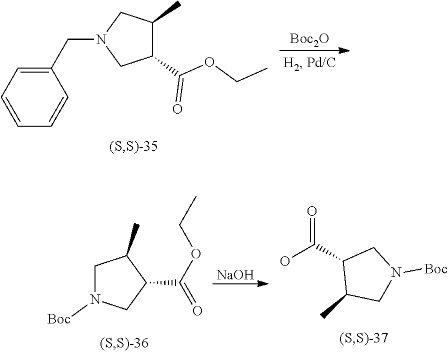

A solution of (S,S)-35 (12.80 g, 51.8 mmol) and Boc₂O (13.57 g, 1.2 eq) in EtOH (150 mL) was placed in an autoclave under N2-protective atmosphere and Pd/C (5%, 2.56 g) was added. The reaction mixture was hydrogenated with stirring at 45-50° C. at 15-20 Bar H₂ pressure until no more hydrogen was absorbed (48 hrs). The reaction mixture was cooled to RT and filtered, and the filter was washed with EtOH (50 mL). The filtrate was evaporated at <45° C. to about 25 mL. Water (10 mL) and NaOH solution (2 mL) was added and the resulting reaction mixture was stirred at RT for 2 hrs (GC analysis showed complete disappearance of the starting material at this point). Water (125 mL) was added and the resulting mixture was extracted with MTBE (2×50 mL). The aqueous phase was treated with 2N HCl solution to achieve a pH value of 3-4 (ca. 25 mL) and the resulting solution was extracted with MTBE (2×150 mL). The combined organic extracts were washed with brine (50 mL) and evaporated to about 20 mL. n-Heptane (40 mL) was added and the resulting reaction mixture was left at 0° C. for 2 hrs after which the solid was filtered off and dried to give the product (S,S)-37 as a solid (9.48 g, 41.7 mmol). The ee at this step was determined to 97.5%. This material had identical NMR and LC/MS properties to rac-37 described below.

### Synthesis of racemic trans-4-methyl-pyrrolidine-1,3-dicarboxylic acid 1-tert-butyl ester (37)

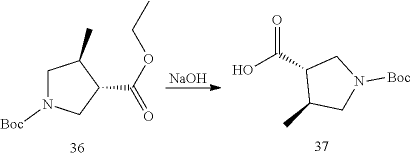

A solution of compound 36 (10.0 g, 39.1 mmol), NaOH (3.10 g, 78.2 mmol) in methanol/H₂O (50/5 mL) was stirred at room temperature for 2 hours. The reaction mixture was concentrated and extracted with EA (150 mL). The aqueous phase was acidified by 2 M HCl at 0° C. to pH ˜5 and extracted with EtOAc (150 mL×3). The combined organic layers were washed with brine, dried and concentrated to afford compound 37 (8.0 g, 90%) as an oil.

¹H NMR (400 MHz, DMSO-d₆): δ 12.43 (s, 1H), 3.55-3.51 (m, 2H), 3.47-3.27 (m, 1H), 2.85-2.78 (m, 1H), 2.63-2.57 (m, 1H), 2.34-2.28 (m, 1H), 1.55 (s, 9H), 1.03 (d, J=4.8 Hz, 3H).

### Synthesis of (S,S)-trans-3-acetyl-4-methyl-pyrrolidine-1-carboxylic acid tert-butyl ester (S,S)-(39) via (S,S)-trans-3-(methoxy-methyl-carbamoyl)-4-methyl-pyrrolidine-1-carboxylic acid tert-butyl ester (S,S)-(38)

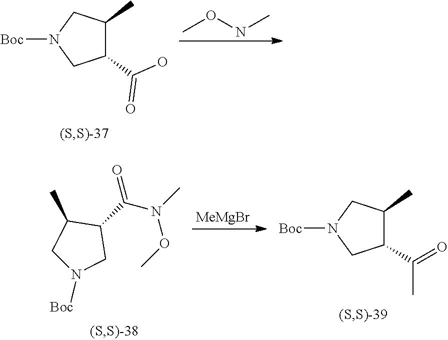

To a solution of (S,S)-37 (5.0 g, 22.0 mmol) in DCM (50 mL) was added CDI (4.25 g, 1.2 eq) over 10 mins while keeping the temperature below 5° C. throughout. The reaction mixture was stirred for 1 hr after which N,O-dimethylhydroxylamine hydrochloride (3.0 g, 1.4 eq) was added in small portions over about 10 mins keeping the temperature below 5° C. The reaction was then allowed to warm to room temperature and stirred for 12 hrs at which the starting material had been fully consumed. Water (50 mL) was added, the phases were separated and the aq phase was extracted with DCM (35 mL). The combined organic phases were washed with water (50 mL) and concentrated to about 5 mL. THE (20 mL) was added and the resulting solution was evaporated to dryness and dried in high vacuum. Dry THE (50 mL) was added, the solution was cooled to 0° C. and MeMgCl (3 M, 11.35 mL, 1.5 eq) was added dropwise under an N₂ atmosphere over 30 mins making sure to maintain the temperature below 5° C. The reaction mixture was then heated to RT and stirred for 2 hrs (at this point the Weinreb amide had been completely converted). Saturated aq. ammonium chloride (50 mL) was added dropwise below 25° C. to quench the reaction and the resulting reaction mixture was extracted with EtOAc (2×50 mL), and the combined organic extracts were washed with brine (50 mL) and evaporated to about 5 mL. THE (25 mL) was added and the resulting solution was evaporated to dryness in vacuo to give the product (S,S)-39 as an oil (4.91 g, 21.6 mmol) in about 98% ee. All spectral properties were identical to those of rac-39.

### Synthesis of racemic trans-3-(methoxy-methyl-carbamoyl)-4-methyl-pyrrolidine-1-carboxylic acid tert-butyl ester (38)

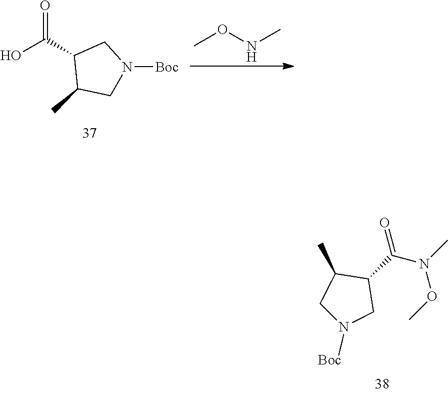

To a solution of 37 (8.0 g, 34.9 mmol) and O,N-dimethyl-hydroxylamine (4.0 g, 41.9 mmol) in DCM (50 mL) was added CDI (6.8 g, 41.9 mmol). The mixture reaction was stirred at 20° C. for 18 hours. To the mixture solution was added water (100 mL) and extracted with DCM (100 mL×3). The combined organic layers were washed with brine (30 mL), dried and concentrated in vacuo. The crude product was purified by flash chromatography (PE/EtOAc=20/1) to afford racemic trans 38 (8.0 g, 84% yield) as an oil.

¹H NMR (400 MHz, DMSO-d₆): δ 3.68 (s, 3H), 3.60-3.48 (m, 2H), 3.20-3.05 (m, 5H), 2.84-2.73 (m, 1H), 2.40-2.32 (m, 1H), 1.39 (s, 9H), 0.96 (d, J=4.8 Hz, 3H).

### Synthesis of racemic trans-3-acetyl-4-methyl-pyrrolidine-1-carboxylic acid tert-butyl ester (39)

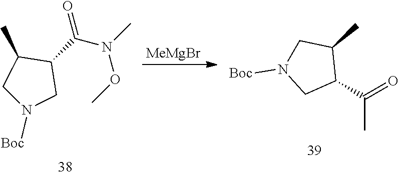 [SMILES: "MeMgBr" ; CON(C)C(=O)[C@@H]1CN(C(=O)OC(C)(C)C)C[C@H]1C ; CC(=O)[C@@H]1CN(C(=O)OC(C)(C)C)C[C@H]1C]

To a solution of 38 (8.0 g, 29.4 mmol) in THE (60 mL) was added MeMgBr (3.0 M, 13 mL, 38.2 mmol) at 0° C. The reaction mixture was stirred at room temperature for 2 hours. The mixture reaction was quenched with saturated NH₄Cl aqueous solution (200 mL) and extracted with EtOAc (300 mL×3). The combined organic layers were washed with brine, dried and concentrated in vacuo. The crude product was purified by flash chromatography (PE/EtOAc=10/1) to afford the desired racemic trans 39 (6.0 g, 94% yield) as an oil.

¹H NMR (400 MHz, DMSO-d₆): δ 3.66-3.51 (m, 1H), 3.49-3.39 (m, 1H), 3.34-3.24 (m, 1H), 2.88-2.79 (m, 2H), 2.34-2.30 (m, 1H), 2.15 (s, 3H), 1.36 (s, 9H), 1.02-1.00 (m, 3H).

### Synthesis racemic trans-3-(2-bromo-acetyl)-4-methyl-pyrrolidine-1-carboxylic acid tert-butyl ester (40)

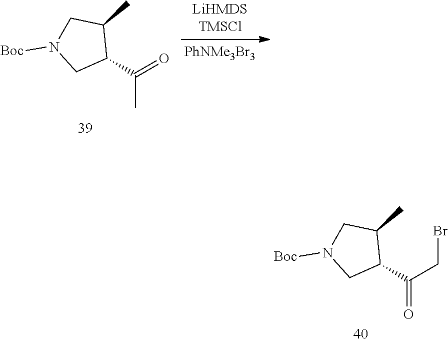 [SMILES: CC(=O)[C@@H]1CN(C(=O)OC(C)(C)C)C[C@H]1C ; C[C@@H]1CN(C(=O)OC(C)(C)C)C[C@H]1C(=O)CBr]

A solution of LiHMDS (1M in THF, 40 mL, 40 mmol) was added to the solution of 39 (6.0 g, 26.4 mmol) in THE (100 mL) under an N₂ atmosphere at −78° C. The reaction mixture was stirred at this temperature for one hour. Then TMSCl (10 mL, 26.4 mmol) was added dropwise at −78° C. and the reaction temperature was raised to 0° C. After one hour, PhMe₃NBr₃ (11.0 g, 29.1 mmol) was added at 0° C. The mixture reaction was stirred for another an hour, then stirred at room temperature overnight. The reaction was quenched with water (200 mL) and extracted with EtOAc (250 mL×3). The combined organic layers were washed with brine, dried and concentrated in vacuo. The crude product was purified by flash chromatography (PE/EtOAc=10/1) to afford the desired racemic trans 40 (4.5 g, 56% yield) as an oil.

¹H NMR (400 MHz, CDCl₃): δ 4.05 (s, 2H), 3.69-3.50 (m, 2H), 3.36-3.30 (m, 1H), 3.04-2.86 (m, 2H), 2.51-2.43 (m, 1H), 1.39 (s, 9H), 1.10-1.05 (m, 3H).

### Synthesis (S,S)-trans-3-(2-bromo-acetyl)-4-methyl-pyrrolidine-1-carboxylic acid tert-butyl ester (S,S)-(40)

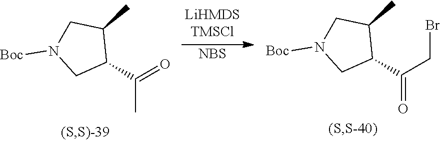

A solution of LiHMDS (1M in THF, 21.12 mL, 21.12 mmol) was added dropwise to a solution of (S,S)-39 (3.96 g, 17.4 mmol) in THE (50 mL) under an N₂ atmosphere at −78° C. The reaction mixture was stirred at this temperature for one hour. Then TMSBr (6.43 g, 42 mmol) was added dropwise at −78° C. and the reaction temperature was allowed to warm to 0° C. After one hour NBS (2.76 g, 15.5 mmol) was added in small portions at 0° C. TLC showed that all starting material had been consumed. Water (20 mL) was added dropwise keeping the temperature at RT and the resulting reaction mixture was stirred for 30 mins. The phases were separated and the aq phase was extracted with MTBE (2×15 mL). The combined organic phases were washed with brine, dried and concentrated in vacuo. The residue was redissolved in MTBE (25 mL), washed with water (3×10 mL) and brine (10 mL), and concentrated in vacuo to give the product as an oil which could be purified by flash chromatography (PE/EtOAc=10/1) to afford the desired (S,S)-40 (6.4 g, 20.9 mmol) as an oil.

### Synthesis of racemic trans-2,5-dibromo-3-[2-(1-tert-butoxycarbonyl-4-methyl-pyrrolidin-3-yl)-2-oxo-ethyl]-3H-imidazole-4-carboxylic acid methyl ester (41)

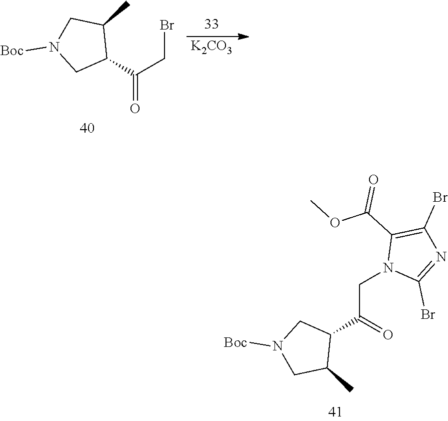

To a solution of 33 (4.1 g, 14.7 mmol) in DMF (30 mL) was added K₂CO₃ (5.8 g, 42.5 mmol). After stirring for 15 minutes, compound 40 (4.5 g, 14.7 mmol) was added to the reaction mixture. The reaction was stirred at room temperature for 5 hours. The reaction mixture was diluted with EtOAc (200 mL), washed with brine (200 mL×2). Then the organic phase was dried (Na₂SO₄), filtered and concentrated in vacuo. The residue was purified by column chromatography (PE/EtOAc=10/0-3/1) to afford racemic trans 41 (3.0 g, 40% yield) as a solid.

¹H NMR (400 MHz, DMSO-d₆): 5.41 (s, 2H), 3.78 (s, 3H), 3.68-3.66 (m, 1H), 3.48-3.45 (m, 1H), 3.34-3.31 (m, 1H), 3.20-3.25 (m, 1H), 2.92-2.87 (m, 1H), 2.50-2.46 (m, 1H), 1.36 (s, 9H), 1.07 (m, 3H).

### Synthesis of (S,S)-trans-2,5-dibromo-3-[2-(1-tert-butoxycarbonyl-4-methyl-pyrrolidin-3-yl)-2-oxo-ethyl]-3H-imidazole-4-carboxylic acid methyl ester (S,S)-(41)

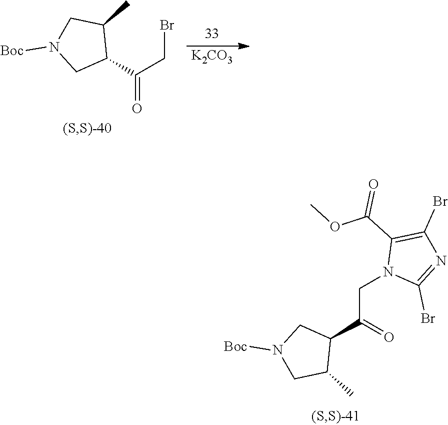

To a solution of 33 (2.78 g, 9.79 mmol) in NMP (30 mL) was added Na₂CO₃ (3.11 g, 26.2 mmol). After stirring for 15 minutes, compound (S,S)-40 (4.5 g, 14.7 mmol) was added to the reaction mixture. The reaction was stirred at room temperature for 5 hours. The reaction mixture was diluted with EtOAc (200 mL), washed with brine (200 mL×2). Then the organic phase was dried (Na₂SO₄), filtered and concentrated in vacuo. The residue was purified by column chromatography (PE/EtOAc=10/0-3/1) to give the product as a crude solid, which was recrystallized from 2-propanol/n-heptane to give (S,S)-41 (3.03 g, 40% yield) as a solid. The ee of the material at this stage was determined to be above 99%. All spectral data were identical to those of rac-41.

### Synthesis of racemic trans-3-(1,3-dibromo-8-oxo-7,8-dihydro-imidazo[1,5-a]pyrazin-6-yl)-4-methyl-pyrrolidine-1-carboxylic acid tert-butyl ester (42)

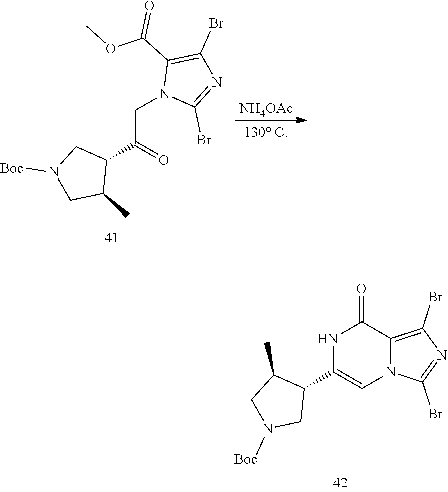 [SMILES: COC(=O)c1c(Br)nc(Br)n1CC(=O)[C@H]1CN(C(=O)OC(C)(C)C)C[C@@H]1C ; C[C@@H]1CN(C(=O)OC(C)(C)C)C[C@H]1c1cn2c(Br)nc(Br)c2c(=O)[nH]1 ; "NH4OAc"]

To a solution of 41 (3.0 g, 5.89 mmol) in MeOH (150 mL) was added NH₄OAc (9.07 g, 117.8 mmol). The reaction mixture was heated to 130° C. in a pressure vessel for 15 hours. The reaction mixture was filtered and concentrated to get the crude product. The residue was purified by column chromatography (DCM/MeOH=100/1˜10/1) to afford racemic trans 42 (2.2 g, 80% yield) as a solid.

¹H NMR (400 MHz, DMSO-d₆): 10.98 (br. s, 1H), 7.10 (s, 1H), 3.63-3.54 (m, 2H), 3.39-3.34 (m, 1H), 2.84-2.77 (m, 2H), 2.50 (m, 1H), 1.41 (s, 9H), 0.96 (m, 3H).

### Synthesis of (S,S)-trans-3-(1,3-dibromo-8-oxo-7,8-dihydro-imidazo[1,5-a]pyrazin-6-yl)-4-methyl-pyrrolidine-1-carboxylic acid tert-butyl ester (S,S)-(42)

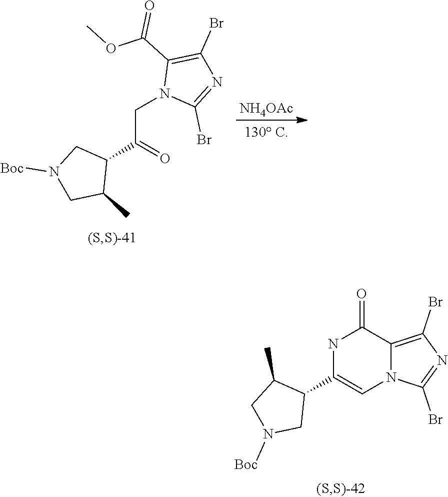 [SMILES: COC(=O)c1c(Br)nc(Br)n1CC(=O)[C@H]1CN(C(=O)OC(C)(C)C)C[C@@H]1C ; C[C@@H]1CN(C(=O)OC(C)(C)C)C[C@H]1c1cn2c(Br)nc(Br)c2c(=O)[nH]1 ; "NH4OAc"]

To a solution of (S,S)-41 (3.03 g, 5.9 mmol) in 2-propanol (20 mL) was added NH₄OAc (9.18 g, 118 mmol). The reaction mixture was heated at 105-110° C. for 12 hrs after which it was poured into water (60 mL) with stirring and left for two hrs. The reaction mixture was filtered and concentrated to get the crude product. The residue was purified by column chromatography (DCM/MeOH=100/1-10/1) and evaporated to afford (S,S)-42 (2.1 g, 4.4 mmol) as a solid. The material was determined to have 99.3% ee and similar spectral properties to those of rac-42.

### Synthesis of racemic trans-3-[1-bromo-3-(3,6-dihydro-2H-pyran-4-yl)-8-oxo-7,8-dihydro-imidazo[1,5-a]pyrazin-6-yl]-4-methyl-pyrrolidine-1-carboxylic acid tert-butyl ester (43)

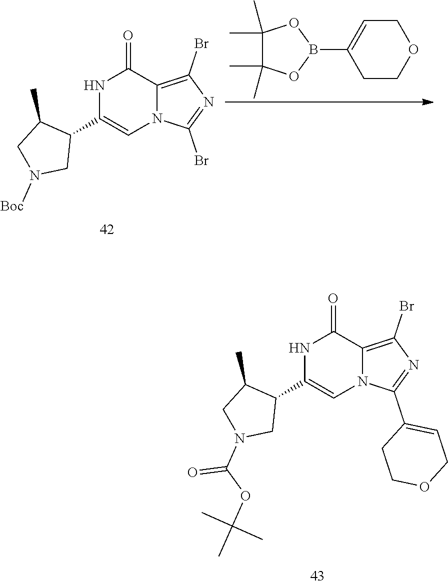

To a mixture of compound 42 (2.2 g, 4.62 mmol) and 4-(4,4,5,5-tetramethyl-[1,3,2]dioxaborolan-2-yl)-3,6-dihydro-2H-pyran (1.1 g, 5.08 mmol) in THE (200 mL) was added potassium phosphate (2.7 g, 13.86 mmol). The reaction mixture was degassed by purging with N₂ for 5 min, before Pd₂(dba)₃ (0.8 g, 0.92 mmol) and Xanthphos (1.0 g, 1.84 mmol) were added to the mixture. The resulting suspension was degassed with N₂ for 10 minutes. Then the mixture reaction was heated to 80° C. under an N2 atmosphere for 15 hours. After cooling to room temperature, the reaction mixture was diluted with EtOAc (250 mL) and the precipitate was filtered off. The filtrate was concentrated. The crude residue was purified by column chromatography on silica gel (eluting with EtOAc) to afford 43 (1.3 g, 60% yield) as a solid.

¹H NMR (400 MHz, DMSO-d₆): δ 10.80 (m, 1H), 7.34 (s, 1H), 6.42 (s, 1H), 4.30-4.29 (m, 2H), 3.92-3.80 (m, 2H), 3.63-3.33 (m, 4H), 2.87-2.71 (m, 2H), 2.50 (m, 1H), 1.41 (s, 9H), 0.95 (m, 3H).

### Synthesis of (S,S)-trans-3-[1-bromo-3-(3,6-dihydro-2H-pyran-4-yl)-8-oxo-7,8-dihydro-imidazo[1,5-a]pyrazin-6-yl]-4-methyl-pyrrolidine-1-carboxylic acid tert-butyl ester (S,S)-(43)

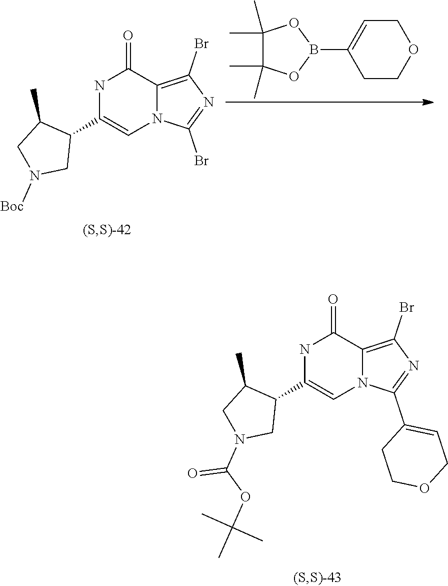

To a mixture of compound (S,S)-42 (2.11 g, 4.42 mmol) and 4-(4,4,5,5-tetramethyl-[1,3,2]dioxaborolan-2-yl)-3,6-dihydro-2H-pyran (0.975 g, 4.64 mmol) in 1,4-Dioxane (40 mL) and water (10 mL) was added potassium phosphate (2.57 g, 12.2 mmol). The reaction mixture was degassed by purging with N₂ for 5 min, before Pd₂(dba)₃ (0.8 g, 0.9 mmol) and Xanthphos (1.0 g, 1.8 mmol) were added to the mixture. The resulting suspension was degassed with N₂ for 10 minutes. Then the mixture reaction was heated to 80° C. under an N2 atmosphere for 15 hours. After cooling to room temperature, the reaction mixture was diluted with EtOAc (250 mL) and the solid was removed by filtration through Celite. The filtrate was concentrated. The crude residue was purified by column chromatography on silica gel (eluting with EtOAc) to afford 43 (1.4 g, 2.92 mmol) as a solid. The material has an ee above 99% at this stage.

### Synthesis of racemic trans-3-methyl-4-[8-oxo-3-(tetrahydro-pyran-4-yl)-7,8-dihydro-imidazo[1,5-a]pyrazin-6-yl]-pyrrolidine-1-carboxylic acid tert-butyl ester (44)

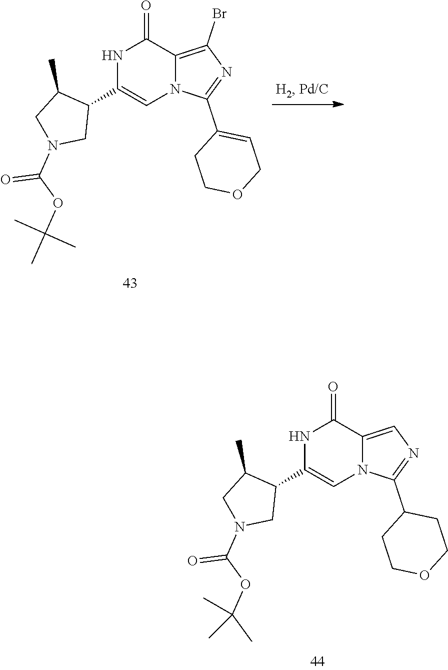 [SMILES: C[C@@H]1CN(C(=O)OC(C)(C)C)C[C@H]1c1cn2c(C3=CCOCC3)nc(Br)c2c(=O)[nH]1 ; C[C@@H]1CN(C(=O)OC(C)(C)C)C[C@H]1c1cn2c(C3CCOCC3)ncc2c(=O)[nH]1]

To a solution of 43 (1.3 g, 2.73 mmol) in DMF (100 mL) and methanol (30 mL) was added 10% Pd/C (0.8 g). The flask was charged with hydrogen (50 psi) and the mixture was stirred at 50° C. overnight. After cooling down, the reaction mixture was filtered through Celite. The filtrate was concentrated under reduced pressure. The crude product was purified by column chromatography on silica gel (eluting with DCM/CH₃OH=100/1-20/1) to afford compound 44 (0.99 g, 90% yield) as a solid.

¹H NMR (400 MHz, CDCl₃): 10.80 (br d, 1H), 7.86 (s, 1H), 6.79 (s, 1H), 4.13-4.10 (m, 2H), 3.83-3.79 (m, 3H), 3.63-3.49 (m, 2H), 3.13-3.03 (m, 2H), 2.77-2.75 (m, 2H), 2.54-2.53 (m, 1H), 2.11-2.06 (m, 2H), 1.80-1.85 (m, 2H), 1.48 (m, 9H), 1.12 (d, J=6.4 Hz, 3H).

### Synthesis of (S,S)-trans-3-methyl-4-[8-oxo-3-(tetrahydro-pyran-4-yl)-7,8-dihydro-imidazo[1,5-a]pyrazin-6-yl]-pyrrolidine-1-carboxylic acid tert-butyl ester (S,S)-(44)

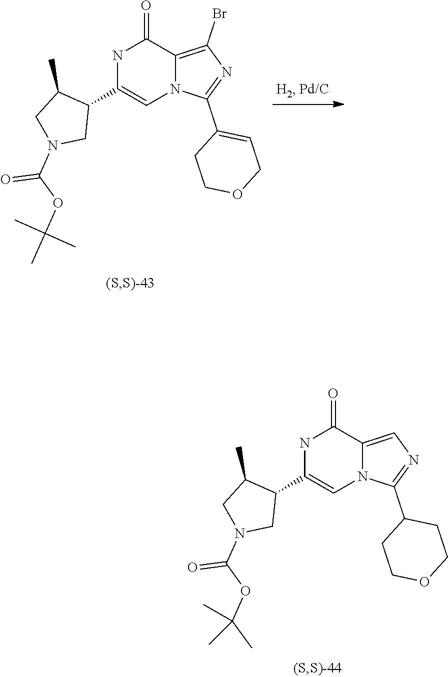 [SMILES: C[C@@H]1CN(C(=O)OC(C)(C)C)C[C@H]1c1cn2c(C3=CCOCC3)nc(Br)c2c(=O)[nH]1 ; C[C@@H]1CN(C(=O)OC(C)(C)C)C[C@H]1c1cn2c(C3CCOCC3)ncc2c(=O)[nH]1]

A solution of (S,S)-43 (1.15 g, 2.41 mmol) in methanol (50 mL) was placed in an autoclave under N2-protective atmosphere and 10% Pd/C (0.8 g) was added under a nitrogen atmosphere. The reaction mixture was hydrogenated with stirring at 45-50° C. at 10-15 Bar H₂ pressure until no more hydrogen was absorbed (24 hrs). After cooling down, the reaction mixture was filtered through Celite. The filtrate was concentrated under reduced pressure. The crude product was purified by column chromatography on silica gel (eluting with DCM/CH₃OH=100/1-20/1) to afford compound 44 (0.97 g, 2.41 mmol) as a solid. The ee was determined to be above 99%.

### Synthesis of racemic trans-6-(4-methyl-pyrrolidin-3-yl)-3-(tetrahydro-pyran-4-yl)-7H-imidazo[1,5-a]pyrazin-8-one (45)

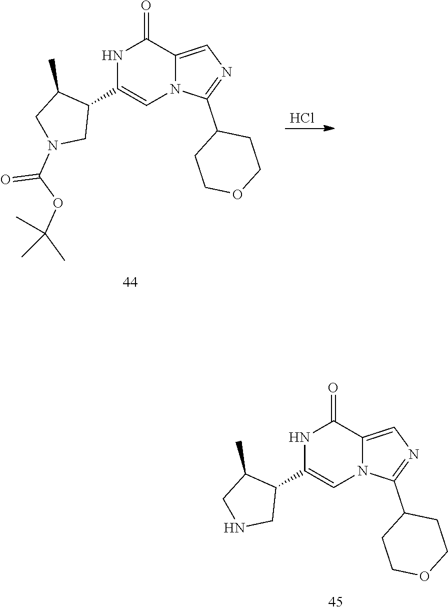 [SMILES: C[C@@H]1CN(C(=O)OC(C)(C)C)C[C@H]1c1cn2c(C3CCOCC3)ncc2c(=O)[nH]1 ; Cl ; C[C@@H]1CNC[C@H]1c1cn2c(C3CCOCC3)ncc2c(=O)[nH]1]

To a solution of compound 44 (0.99 g, 2.49 mmol) in CH₂C₂ (20 mL) was added HCl/Et₂O solution (20 mL). The resulting mixture was stirred at room temperature for 2 hours. The reaction was concentrated in vacuo to afford racemic trans 45 hydrochloride (0.75 g, 100% yield) as a solid.

¹H NMR (400 MHz, DMSO-d₆): δ 11.47 (s, 1H), 9.93 (s, 2H), 8.41 (s, 1H), 7.92 (s, 1H), 3.98-3.95 (m, 2H), 3.85-3.80 (m, 1H), 3.58-3.44 (m, 3H), 2.97-2.88 (m, 2H), 2.60-2.50 (m, 3H), 1.98-1.78 (m, 4H), 1.08 (m, 3H).

### Synthesis of (S,S)-trans-6-(4-methyl-pyrrolidin-3-yl)-3-(tetrahydro-pyran-4-yl)-7H-imidazo[1,5-a]pyrazin-8-one (S,S)-(45)

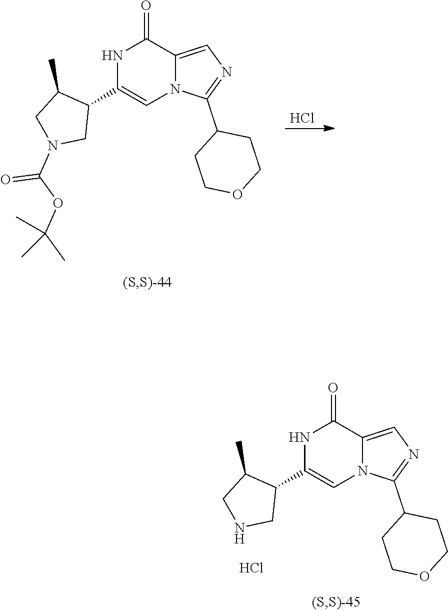 [SMILES: C[C@@H]1CN(C(=O)OC(C)(C)C)C[C@H]1c1cn2c(C3CCOCC3)ncc2c(=O)[nH]1 ; Cl ; C[C@@H]1CNC[C@H]1c1cn2c(C3CCOCC3)ncc2c(=O)[nH]1 ; Cl]

To a solution of compound (S,S)-44 (800 mg, 2.0 mmol) was added to a cold (0° C.) solution of HCl in MeOH (1.5 M, 10 mL) and the resulting reaction mixture was stirred while being allowed to reach room temperature. After stirring for 2 hrs the reaction was concentrated in vacuo to afford (S,S)-45 hydrochloride (0.60 g, 2.0 mmol) as a solid.

¹H NMR (400 MHz, DMSO-d₆): δ 11.47 (s, 1H), 9.93 (s, 2H), 8.41 (s, 1H), 7.92 (s, 1H), 3.98-3.95 (m, 2H), 3.85-3.80 (m, 1H), 3.58-3.44 (m, 3H), 2.97-2.88 (m, 2H), 2.60-2.50 (m, 3H), 1.98-1.78 (m, 4H), 1.08 (m, 3H).

### Synthesis of racemic trans-6-(4-methyl-1-pyrimidin-2-ylmethyl-pyrrolidin-3-yl)-3-(tetrahydro-pyran-4-yl)-7H-imidazo[1,5-a]pyrazin-8-one (P3)

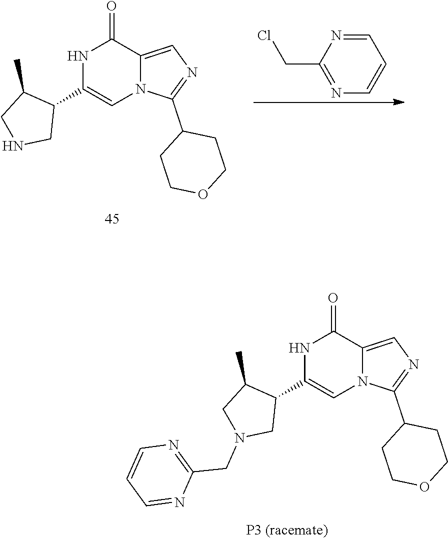 [SMILES: C[C@@H]1CNC[C@H]1c1cn2c(C3CCOCC3)ncc2c(=O)[nH]1 ; ClCc1ncccn1 ; C[C@@H]1CN(Cc2ncccn2)C[C@H]1c1cn2c(C3CCOCC3)ncc2c(=O)[nH]1]

To a solution of compound 45 (0.75 g, 2.49 mmol), 2-chloromethyl-pyrimidine (0.49 g, 2.99 mmol) in DMF (10 mL) and CH₃CN (30 mL) was added K₂CO₃ (1.7 g, 12.5 mmol). The mixture was stirred at 45° C. for 48 hours. The reaction mixture was filtered, concentrated in vacuo. The residue was purified by flash column chromatography (gradient elution from DCM to 15% MeOH in DCM) to afford racemic trans P3 (580 mg, 59% yield) as a solid.

¹H NMR (400 MHz, CD₃OD): δ 8.85 (d, J=4.8 Hz, 2H), 7.79 (s, 1H), 7.42 (t, J=4.8 Hz, 1H), 7.36 (s, 1H), 4.11-4.04 (m, 3H), 3.93 (d, J=15.2 Hz, 1H), 3.684-3.62 (m, 2H), 3.41-3.32 (m, 2H), 3.16-3.13 (m, 1H), 2.85-2.80 (m, 2H), 2.44-2.40 (m, 1H), 2.28-2.23 (m, 1H), 2.04-1.86 (m, 4H), 1.17 (d, J=6.4 Hz, 3H). MS Calcd.: 394.5; MS Found: 395.8 ([M+H]⁺).

The racemic mixture of P3 (1.4 g) was separated by Chiral HPLC (Column: Chiralpak IA, 250×4.6 mm×Sum; mobile phase Hex/EtOH/DEA=70:30:0.2) with a flow rate of 1.0 mL/min, to afford P3 enantiomer 1 (i.e., Compound P3.1, (3S,4S)-6-(4-methyl-1-pyrimidin-2-ylmethyl-pyrrolidin-3-yl)-3-(tetrahydro-pyran-4-yl)-7H-imidazo[1,5-a]pyrazin-8-one) (0.52 g, RT=9.98 min) and P3 enantiomer 2 ((3R,4R)-6-(4-methyl-1-pyrimidin-2-ylmethyl-pyrrolidin-3-yl)-3-(tetrahydro-pyran-4-yl)-7H-imidazo[1,5-a]pyrazin-8-one, opposite of P3 enantiomer 1) (0.49 g, RT=12.6 min).

### Synthesis of (S,S)-trans-6-(4-methyl-1-pyrimidin-2-ylmethyl-pyrrolidin-3-yl)-3-(tetrahydro-pyran-4-yl)-7H-imidazo[1,5-a]pyrazin-8-one (S,S)-(P3)

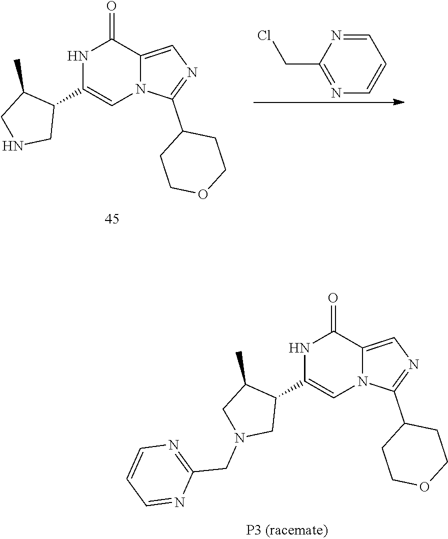 [SMILES: C[C@@H]1CNC[C@H]1c1cn2c(C3CCOCC3)ncc2c(=O)[nH]1 ; ClCc1ncccn1 ; C[C@@H]1CN(Cc2ncccn2)C[C@H]1c1cn2c(C3CCOCC3)ncc2c(=O)[nH]1]

To a solution of compound (S,S)-45 (0.60 g, 2.0 mmol) and 2-chloromethyl-pyrimidine (0.40 g, 2.40 mmol) in DCM (15 mL) was added DIPEA (3.1 g, 24 mmol) and the mixture was stirred at RT for 24 hrs (at this time all the starting material had been converted). The reaction mixture was cooled to 5° C., and deionised water (10 mL) was added. The pH of the aqueous phase was adjusted to pH 6.0 with addition of conc hydrochloric acid (about 1 mL) while keeping the temperature of the mixture <25° C. The phases were allowed to separate and the organic phase was washed with brine (3×5 mL) (these washings were discarded). The aqueous phase was extracted with dichloromethane (10 mL), and the organic phase from this extraction was washed with brine (3×5 mL). The combined organic phases were dried over sodium sulfate (3 g) for 1 hour, filtered and evaporated. The resulting residue was subjected to column chromatography (as described for rac-(P3)) to give (S,S)-P3 (580 mg, 59% yield) as a solid after evaporation. This material has ee above 99% and is identical in all ways to P3 Enantiomer 1 (described above).

### Synthesis of (Aminooxy) (Diphenyl) Phosphine Oxide (B)

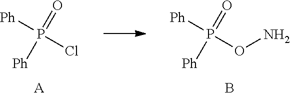

To a suspension of hydroxylamine hydrochloride (73.5 g, 1.05 mol) in dichloromethane (500 mL) was added DIPEA (136 g, 1.05 mol) over 15 minutes at −30° C. under a nitrogen atmosphere. A white precipitate formed upon the addition. After stirring for one hour at that temperature, a solution of diphenylphosphinic chloride A (50 g, 0.2 mol) in dichloromethane (100 mL) was added over 60 minutes. The mixture reaction was warmed to 0° C. over 1 hour with stirring. The reaction was quenched by adding water (200 mL) over 10 minutes. After stirring the mixture for 0.5 hour, the precipitate was collected by filtration and washed with water (100 mL×2). Then the solid was dried under reduced pressure to afford a crude product. The crude product was triturated in EtOH to afford compound B (27 g, 56% yield) as a white solid.

¹HNMR (400 MHz, CD30D): δ77.91-7.79 (m, 5H), 7.62-7.50 (m, 7H).

MS Calcd.: 233; MS Found: 234 ([M+H]⁺).

### Synthesis of 3-amino-3H-imidazole-4-carboxylic acid methyl ester (46)

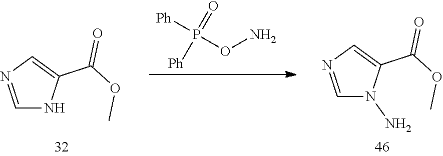

To a solution of compound 3H-Imidazole-4-carboxylic acid methyl ester 32 (30.0 g, 0.24 mol) in THE (1.0 L) was dropwise added LiHMDS (239 mL, 10M in THF, 2.4 mol) over 2 hours at −78° C. Then the reaction mixture was stirred at −78° C. for another two hours and allowed to warm to −10° C. Compound B (60.0 g, 0.26 mol) was added at this temperature. Then the mixture reaction was stirred at ambient temperature overnight. After quenching with water (250 mL), the reaction mixture was concentrated. The crude product was purified by column chromatography on silica gel (DCM/MeOH=20/1) to afford compound 46 (24 g, 73% yield) as a solid.

¹H NMR (400 MHz, DMSO-d6): δ 7.82 (s, 1H), 7.51 (s, 1H), 6.20 (s, 2H), 3.79 (s, 3H). MS Calcd.: 382; MS Found: 383 ([M+H]⁺). MS Calcd.: 141; MS Found: 142 ([M+H]⁺).

### Synthesis of 3-(2-benzyloxy-acetylamino)-3H-imidazole-4-carboxylic acid methyl ester (47)

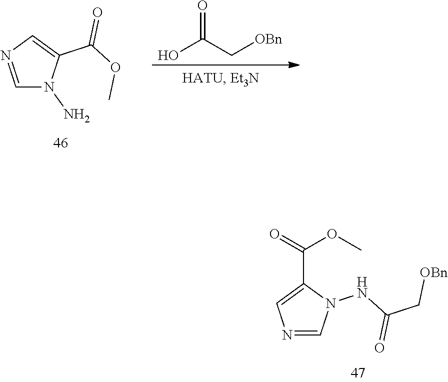

To a solution of compound 46 (4.9 g, 30 mmol), benzyloxy-acetic acid (5.8 g, 30 mmol) and DIPEA (18.6 ml, 90 mmol) in DMF (100 mL) was added HATU (15.8 g, 36 mmol) whilst cooling on an ice-water bath. The mixture was then stirred at ambient temperature overnight. After removal of the solvent, the residue was purified by chromatography on a silica gel column (eluted with PE/EtOAc==10:1 to 2:1) to afford compound 47 (6.1 g, 61% yield) as an oil.

¹H NMR (400 MHz, CDCl₃): δ 9.93 (br. s, 1H), 7.74 (s, 1H), 7.67 (s, 1H), 7.39-7.33 (m, 5H), 4.70 (s, 2H), 4.23 (s, 2H), 3.83 (s, 3H). MS Caled.: 289; MS Found: 300 ([M+H]⁺).

### Synthesis of 3-(2-benzyloxy-acetylamino)-3H-imidazole-4-carboxylic acid amide (48)

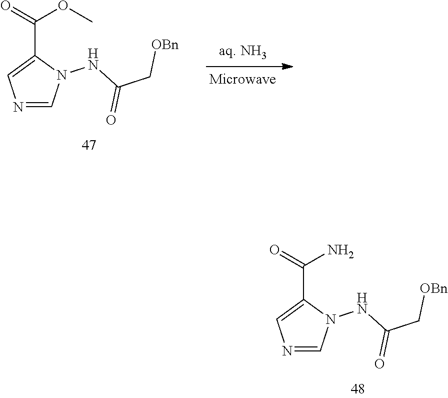 [SMILES: COC(=O)c1cncn1NC(=O)COCc1ccccc1 ; NC(=O)c1cncn1NC(=O)COCc1ccccc1]

Compound 47 (30.0 g, 100 mmol) and conc aq. ammonia (300 mL) were combined in a sealed tube and heated to 70° C. under microwave radiation for 2 hours. The resulting mixture was concentrated in vacuo to afford compound 48 (26.3 g, 96% yield) as a solid. MS Caled.: 274; MS Found: 275 ([M+H]⁺).

### Synthesis of 2-benzyloxymethyl-3H-imidazo[5,1-f][1,2,4]triazin-4-one (49)

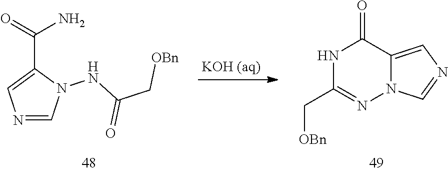 [SMILES: NC(=O)c1cncn1NC(=O)COCc1ccccc1 ; O=c1[nH]c(COCc2ccccc2)nn2cncc12]

To a solution of compound 48 (28.0 g, 100 mmol) in EtOH (240 mL) was dropwise added a solution of KOH (19.8 g, 300 mmol) in water (200 mL). The resulting solution was heated to reflux for 3 hours. After removal of the organic solvent in vacuo, the mixture was poured into ice water and the pH was adjusted to 7.0 with 1M aq IC1 solution. The suspension was filtered off and dried to afford compound 49 (11.3 g, 44.1% yield) as a solid.

¹H NMR (400 MHz, DMSO-d6): δ 12.05 (s, 1H), 8.45 (s, 1H), 7.74 (s, 1H), 7.39-7.29 (m, 5H), 4.59 (s, 2H), 4.36 (s, 2H). MS Calcd.: 256; MS Found: 257 ([M+H]⁺).

### Synthesis of 2-benzyloxymethyl-7-iodo-3H-imidazo[5,1-f][1,2,4]triazin-4-one (50)

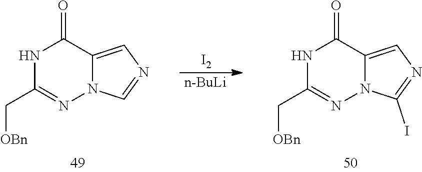

To a solution of compound 49 (10.0 g, 38.2 mmol) in THF (240 mL) was dropwise added n-BuLi (46 mL) at −78° C. and the reaction was stirred below −70° C. for one hour. Iodine (39.3 g, 153 mmol) in THF (120 mL) was added dropwise at this temperature and then the reaction temperature was allowed to warm to room temperature slowly. The reaction was quenched with saturated Na₂SO₃ aqueous solution (120 mL), and then extracted with EtOAc (150 mL×3). The combined organic phases were dried over Na₂SO₄, filtered and concentrated in vacuo to get the crude product. The residue was purified by chromatography on silica gel column (eluted with PE/EtOAc=10:1 to 2:1) to afford compound 50 (4.75 g, 32.5% yield) as a solid.

¹H NMR (400 MHz, DMSO-d6): δ 12.16 (br. s, 1H), 7.84 (s, 1H), 7.42-7.29 (m, 5H), 4.62 (s, 2H), 4.40 (s, 2H). MS Calcd.: 382; MS Found: 383 ([M+H]⁺).

### Synthesis of 2-benzyloxymethyl-7-(3,6-dihydro-2H-pyran-4-yl)-3H-imidazo[5,1-f][1,2,4]triazin-4-one (51)

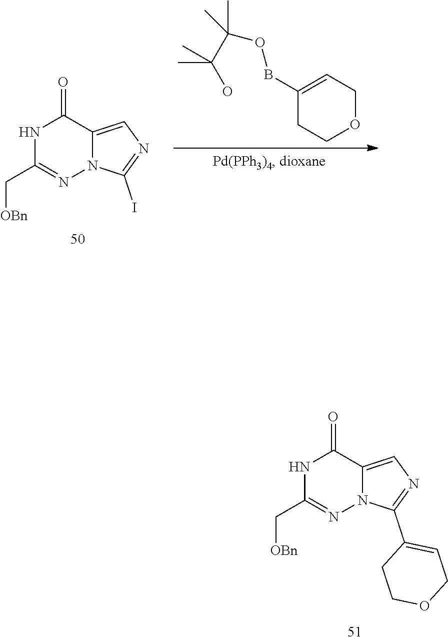

To a solution of compound 50 (4.75 g, 10.0 mmol) in dioxane (80 ml) was dropwise added a solution of Cs₂CO₃ (9.88 g, 30 mmol) in water (12 mL), followed by Pd(PPh₃)₄ (2.36 g, 2.00 mmol) and 4-(4,4,5,5-tetramethyl-[1,3,2]dioxaborolan-2-yl)-3,6-dihydro-2H-pyran (3.86 g, 18.0 mmol). The reaction mixture was degassed by purging with N₂ for 15 min. Then the mixture was heated to reflux for 16 hours. After removal of the solvent in vacuo, the residue was purified by chromatography on a silica gel column (eluted with PE/EtOAc=10:1 to 1:5) to afford compound 51 (2.1 mg, 76% yield) as a solid.

¹H NMR (400 MHz, DMSO-d6): δ 12.10 (br. s, 1H), 7.78 (s, 1H), 7.39-7.30 (m, 5H), 7.25 (s, 1H), 4.62 (s, 2H), 4.41 (s, 2H), 4.27 (d, J=2.8 Hz, 2H), 3.82 (t, J=5.2 Hz, 2H), 2.63 (m, 2H). MS Calcd.: 338; MS Found: 339 ([M+H]⁺).

### Synthesis of 2-hydroxymethyl-7-(tetrahydro-pyran-4-yl)-3H-imidazo[5,1-f][1,2,4]triazin-4-one (52)

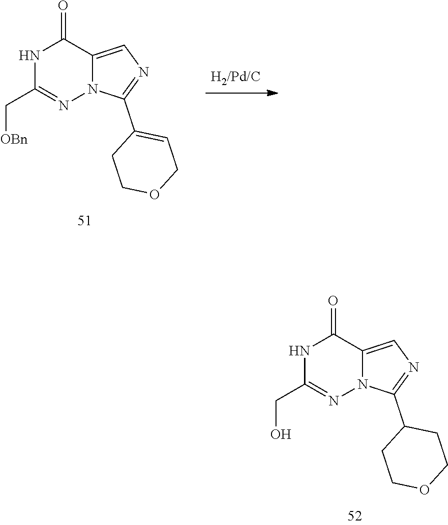 [SMILES: O=c1[nH]c(COCc2ccccc2)nn2c(C3=CCOCC3)ncc12 ; O=c1[nH]c(CO)nn2c(C3CCOCC3)ncc12]

To a solution of compound 51 (1.8 g, 5.0 mmol) in MeOH (70 mL) was added Pd(OH)₂ (20% on Carbon (wetted with ca. 50% Water), 400 mg). The reaction flask was charged with hydrogen (50 psi) and the mixture was stirred on an oil bath heated to 70° C. until LC/MS showed that the starting material had been consumed. The suspension was filtered through celite, the filter was washed with MeOH (100 mL×2) and the combined organic phases were concentrated in vacuo to afford compound 52 (1.0 g, 79% yield) as a solid.

¹H NMR (400 MHz, DMSO-d6): δ 11.65 (s, 1H), 7.68 (s, 1H), 4.30 (s, 2H), 3.96-3.92 (m, 2H), 3.51-3.17 (m, 3H), 1.88-1.81 (m, 4H). MS Calcd.: 250; MS Found: 251 ([M+H]⁺).

### Synthesis of 2-chloromethyl-7-(tetrahydropyran-4-yl)-3H-imidazo[5,1-f][1,2,4]triazin-4-one (53)

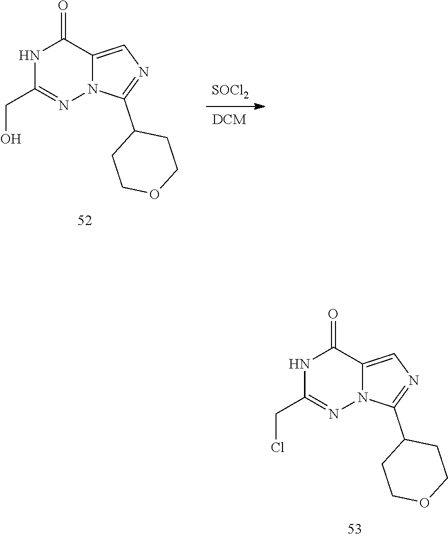

To a solution of compound 52 (1.0 g, 4 mmol) in CH₂C₂ (50 mL) was dropwise added SOCl₂ (15 mL) whilst cooling on an ice-water bath. The resulting mixture was then stirred at ambient temperature overnight. The reaction mixture was concentrated in vacuo to afford compound 53 (107 g, 100% yield) as a solid.

¹H NMR (400 MHz, DMSO-d6): δ 12.50 (br. s, 1H), 8.02 (s, 1H), 4.57 (s, 2H), 3.95 (m, 2H), 3.57-3.48 (m, 3H), 1.91-1.81 (m, 4H). MS Calcd.: 268; MS Found: 269 ([M+H]⁺).

### Synthesis of 3-(4-fluoro-benzyloxy)-azetidine-1-carboxylic acid tert-butyl ester (2)

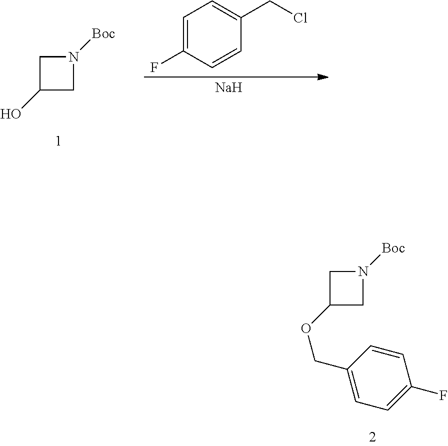

To a solution of compound 3-hydroxy-azetidine-1-carboxylic acid tert-butyl ester 1 (5.30 g, 30 mmol) in DMF (60 mL) was added NaH (1.80 g, 45 mmol) whilst cooling on an ice-water bath. The suspension was then stirred at this temperature for one hour, followed by the addition of 1-choromethyl-4-fluoro-benzene (8.94 g, 60 mmol). The resulting mixture was stirred at ambient temperature overnight. The reaction mixture was poured into water (200 mL) and extracted with EtOAc (150 mL×3). The organic combined phases were dried over Na₂SO₄ filtered and concentrated in vacuo to get the crude product. The residue was purified by chromatography on a silica gel column (eluted with PE/EtOAc=10:1 to 2:1) to afford compound 2 (7.90 g, 94% yield) as an oil.

¹H NMR (300 MHz, DMSO-d6): δ 7.41-7.37 (m, 2H), 7.21-7.14 (m, 2H), 4.40 (s, 2H), 4.33-4.29 (m, 1H), 4.02-3.97 (m, 2H), 3.68-3.66 (m, 2H), 1.37 (s, 9H). MS Calcd.: 281; MS Found: 282 ([M+H]⁺).

### Synthesis of 3-(4-fluoro-benzyloxy)-azetidine (3)

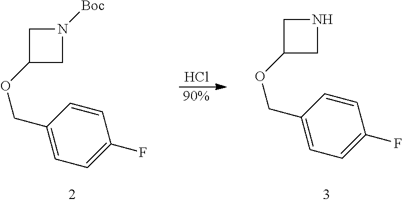

To a solution of compound 2 (2.68 g, 9.30 mmol) in dioxane (30 mL) was added HCl/dioxane (4 M, 9.25 mL) under ice-water bath. The reaction mixture was then stirred at ambient temperature overnight. The reaction solution was concentrated in vacuo to afford compound 3 hydrochloride (1.2 g, 71% yield) as a solid.

¹H NMR (300 MHz, DMSO-d6): δ 7.36 (m, 2H), 7.16 (m, 2H), 4.35 (s, 2H), 4.39 (m, 1H), 3.47 (t, J=7.5 Hz, 2H), 3.38 (t, J=7.2 Hz, 2H). MS Calcd.: 181; MS Found: 182 ([M+H]⁺).

### Synthesis of 2-[3-(4-fluoro-phenoxy)-azetidin-1-ylmethyl]-7-(tetrahydro-pyran-4-yl)-3H-imidazo[5,1-f][1,2,4]triazin-4-one (P4)

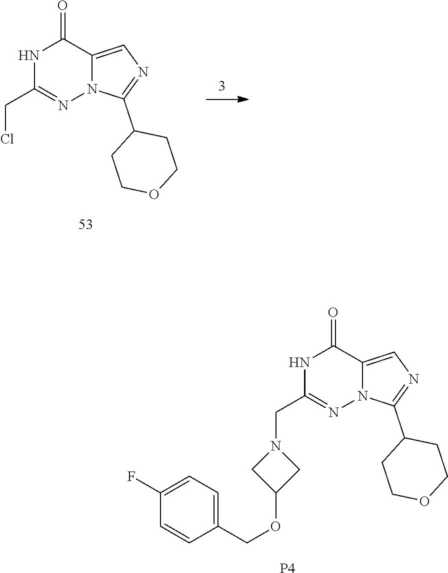

To a solution of compound 53 (1.27 mg, 4.0 mmol) and compound 3 (1.8 g, 8.3 mmol) in CH₃CN (20 mL) was added DIPEA (2.61 mL, 20 mmol). The result solution was heated to 70° C. for 2 hours. TLC indicated that the reaction was complete. The reaction was concentrated in vacuum. The residue was purified by column chromatography on silica gel (eluted with DCM/MeOH 100:1 to 30:1) to afford the desired product P4 (1.23 g, 74% yield) as a solid.

¹H NMR (400 MHz, DMSO-d6): δ 11.70 (br. s, 1H), 7.67 (s, 1H), 7.37 (m, 2H), 7.16 (m, 2H), 4.38 (s, 2H), 4.17 (m, 1H), 3.953.92 (m, 2H), 3.56 (t, J=8.0 Hz, 2H), 3.543.46 (m, 4H), 3.373.35 (m, 1H), 3.063.03 (m, 2H), 1.861.80 (m, 4H). MS Calcd.: 413; MS Found: 414 ([M+H]⁺).

### Example 2. Synthesis and Formulation of Compound P3.1

Compound P3.1 is an enantiomer of P3. Chemical Name: 6-[(3S,4S)-4-methyl-1-(pyrimidin-2-ylmethyl)pyrrolidin-3-yl]-3-tetrahydropyran-4-yl-7H-imidazo[1,5-a]pyrazin-8-one or (3S,4S)-6-(4-methyl-1-pyrimidin-2-ylmethyl-pyrrolidin-3-yl)-3-(tetrahydro-pyran-4-yl)-7H-imidazo[1,5-a]pyrazin-8-one.

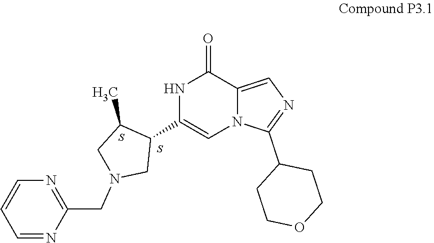

Compound P3.1 was synthesized according to the method in Example 1. The synthesis comprises Suzuki coupling, reduction in the presence of Palladium catalyst, deprotection, and alkylation to produce Compound P3.1.

A stability study has been completed on Compound P3.1. Samples of Compound P3.1 were aliquoted into double-walled polyethylene pouches, which were tied off and then heat-sealed in an aluminum pouch. Samples were stored at ambient temperature and at 40° C.-45° C. (no humidity control) with testing performed over a 3-month period.

There were no changes to appearance or purity of the material at either room temperature or accelerated conditions over the duration of the study, indicating that the drug substance is not readily affected by accelerated temperature conditions.

In another stability study, Compound P3.1 was dissolved at approximately 40 mg/mL of purified water, and evaluated for purity over a period of 8 days. Samples were stored at both refrigerated and ambient conditions, and tested at T=0, Day 2, and Day 8. No significant change to the purity of the compound or appearance of the solution was observed over the course of the study.

In yet another stability study, the study design includes sample storage at both 25° C.±2° C./60% relative humidity (RH) 5% RH, as well as 40° C.±2° C./75% RH±5% RH. Samples are stored in bags comparable to those used for packaging of Compound P3.1. The study is designed to evaluate stability of Compound P3.1 for up to 6 months at the accelerated temperature and for 36 months at the defined storage temperature of 25° C.

Compound P3.1 packaging is prepared by direct filling of the compound into opaque white gelatin capsules (Powder in Capsule, PIC). No binders, bulking agents, or other excipients are added. The capsules contain between 10 and 100 mg of Compound P3.1.

The packaging is monitored in a 6 month to 36 month stability study. The conditions include 25° C./60% RH and 40° C./75% RH (6 months only). Testing includes Appearance, Assay and Related Substances, and Dissolution and Moisture Analysis. A 5° C. arm is also be included, but not tested unless there are indications of product instability at the 25° C. arm of the study.

Alternatively, the dosage form is prepared by blending Compound P3.1 with selected excipients. The excipients that may be used are summarized below in Table 2:

TABLE 2
Proposed Excipients for Future Drug Product Manufacturing

| Excipient | Purpose |
|---|---|
| Pre-gelatinized Starch, NF | Filler, placebo |
| Microcrystalline Cellulose, NF | Filler, placebo |
| Colloidal Silicon Dioxide, NF | Glidant |
| Magnesium Stearate, NF | Lubricant |
| (non-bovine) | |

### Example 3. In Vitro Testing—PDE9 and PDE1 Inhibition Assays

### PDE9 Inhibition Assay

A PDE9 assay may for example, be performed as follows: The assay is performed in 60 uL samples containing a fixed amount of the relevant PDE enzyme (sufficient to convert 20-25% of the cyclic nucleotide substrate), a buffer (50 mM HEPES7.6; 10 mM MgCl₂; 0.02% Tween20), 0.1 mg/ml BSA, 225 pCi of ³H-labelled cyclic nucleotide substrate, tritium labeled cAMP to a final concentration of 5 nM and varying amounts of inhibitors. Reactions are initiated by addition of the cyclic nucleotide substrate, and reactions are allowed to proceed for one hr at room temperature before being terminated through mixing with 15 uL 8 mg/mL yttrium silicate SPA beads (Amersham). The beads are allowed to settle for one hr in the dark before the plates are counted in a Wallac 1450 Microbeta counter. The measured signal can be converted to activity relative to an uninhibited control (100%) and IC₅₀ values can be calculated using the Xlfit extension to EXCEL.

In the context of the present invention the assay was performed in 60 uL assay buffer (50 mM HEPES pH 7.6; 10 mM MgCl₂; 0.02% Tween20) containing enough PDE9 to convert 20-25% of 10 nM ³H-cAMP and varying amounts of inhibitors. Following a 1 hour incubation the reactions were terminated by addition of 15 uL 8 mg/mL yttrium silicate SPA beads (Amersham). The beads were allowed to settle for one hr in the dark before the plates were counted in a Wallac 1450 Microbeta counter. IC₅₀ values were calculated by nonlinear regression using XLfit (IDBS).

Results of the experiments showed that the tested compounds of the invention inhibit the PDE9 enzyme with IC₅₀ values below 100 nM.

### PDE1 Inhibition Assay

PDE1 assays were performed as follows: the assays was performed in 60 μL samples containing a fixed amount of the PDE1 enzym1 (sufficient to convert 20-25% of the cyclic nucleotide substrate), a buffer (50 mM HEPES pH 7.6; 10 mM MgCl₂; 0.02% Tween20), 0.1 mg/ml BSA, 15 nM tritium labelled cAMP and varying amounts of inhibitors. Reactions were initiated by addition of the cyclic nucleotide substrate, and reactions were allowed to proceed for 1 h at room temperature before being terminated through mixing with 20 μL (0.2 mg) yttrium silicate SPA beads (PerkinElmer). The beads were allowed to settle for 1 h in the dark before the plates were counted in a Wallac 1450 Microbeta counter.

The measured signals were converted to activity relative to an uninhibited control (100%) and IC₅₀ values were calculated using XlFit (model 205, IDBS).

### Example 4. In Vitro Pharmacology—cGMP Levels

Effect of Compound P3.1 vs. Hydroxyurea on cGMP in Cultured K562 Cells

This study assessed the effects of Compound P3.1 and HU on the production of cGMP by cultured K562 erythroleukemic cells. Hydroxyurea was included as a comparator as it is currently the only FDA approved treatment for this disease (Charache et al., N Engl J Med., 332(20):1317-22. (1995)). One of the proposed mechanisms of action of HU in SCD is that it may generate NO and modulation of intracellular levels of the NO second messenger, cGMP, may represent an effective and cell-specific approach for amplifying intracellular NO-dependent signaling.

K562 cells were cultured at 37° C. in IMDM® to which the required concentrations of test items or dimethyl sulfoxide (DMSO; negative Control) had been added. Plated cells were maintained at 37° C. for 16 hours at the end of which the amount of cGMP was detected by enzyme immunoassay.

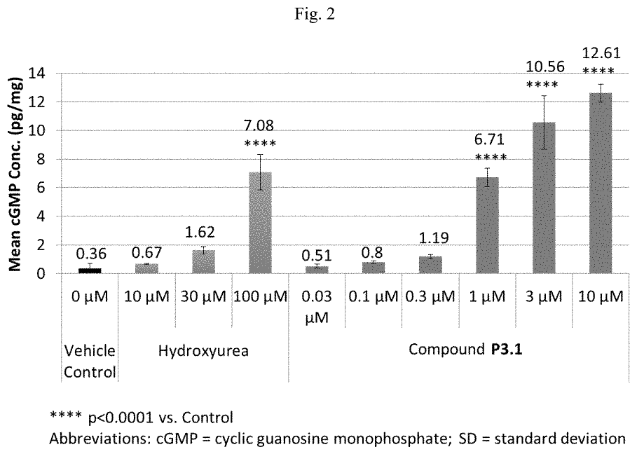

As shown in FIG. 2, treatment with either Compound P3.1 or HU produced dose-dependent and statistically significant increases in cGMP levels. The greatest increase in cGMP was elicited by Compound P3.1 at 10 μM (to 12.61 pg/mg, p>0.0001). Of note, treatment with 1 μM Compound P3.1 increased the concentration of cGMP to approximately the same value elicited by 100 μM HU.

Effect of Compound P3.1 vs. Hydroxyurea on HbF in Cultured K562 Cells

This study assessed the effects of Compound P3.1 and HU on the percentage of cultured K562 erythroleukemic cells positive for fetal hemoglobin (HbF). K562 cells were cultured at 37° C. in IMDM® to which the required concentrations of test items or DMSO (negative control) had been added. Plated cells were maintained at 37° C. for 3 days at the end of which the presence of HbF within cells was detected by flow cytometry.

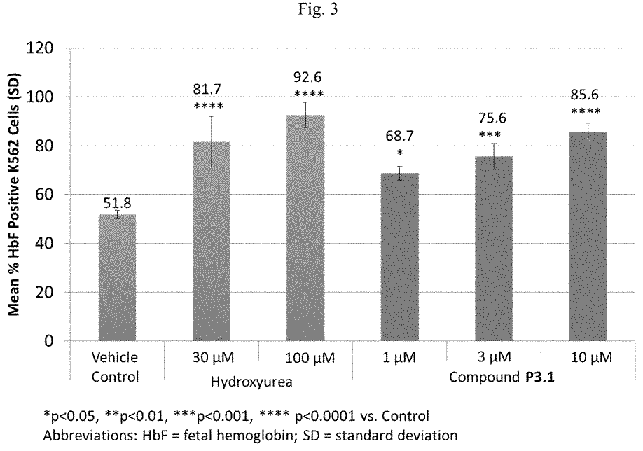

Treatment with either Compound P3.1 or HU produced statistically significant increases in HbF positive cells (FIG. 3). Compared to vehicle treated cells, Compound P3.1 elicited statistically significant increases in HbF cells to 68.7% and 75.6% at concentrations of 1 and 3 M, respectively, with a peak increase to 85.6% at 10 μM. In comparison, HU elicited concentration dependent and statistically significant increases in HbF-positive cells at concentrations of 10 μM and above (but not at 1 and 3 μM), with the highest percentage of HbF-positive cells observed (92.6%) at the 100 μM concentration, which is 10 times the concentration of Compound P3.1 required to elicit a similar response. Consistent results were observed in a similar experiment in which a broader range of concentrations was examined.

Effect of Compound P3.1 vs. Hydroxyurea on HbF Production in CD34+ Derived Red Blood Cells from SCD Subjects

This study assessed the effects of Compound P3.1 and HU on the production of HbF in red blood cells (RBCs) of 5 subjects with SCD. Blood derived CD34+ cells from 5 SCD subjects undergoing transfusions were cultured for 5 days under continuous exposure to either 10 μM Compound P3.1 or 30 μM HU and both the percentage of HbF positive cells and the amount of HbF within the cells were measured.

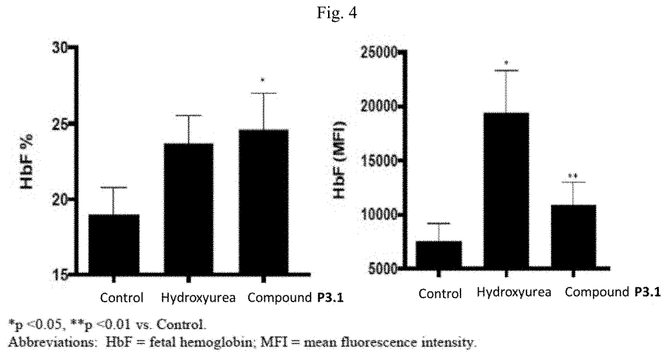

As shown in FIG. 4, Compound P3.1 significantly increased the percentage of HbF positive CD36+ cells relative to control treated cells, from a mean of 18.9% in controls to a mean of 24.6%, and the amount of HbF within these cells, from a mean MFI of 7,484 in controls to 10,840 (145%).

Hydroxyurea elicited greater than 80% cell death in cultures from 2 of the 5 subjects such that no assessments could be made for them. For the remaining 3 subjects, HU did not significantly increase the percentage of HbF positive CD36+ cells (mean of 23.9%) relative to control-treated cells, but it did significantly increase the amount of HbF expressed from a mean MFI of 7,484 in controls to 19,383 (258%).

### Example 5. In Vivo Testing—Blood Brain Barrier Penetration

Male CD mice (20-24 g) were housed pair-wise with free access to food and water for an acclimatization period of 3-7 days before initiation of experiments. Prior to dosing the animals were fasted overnight. During testing, mice were kept in individual cages. The brain-to-plasma distribution was assessed 30 minutes and 2 hours after subcutaneous administration of the test compound at a dose of 10 mg/kg (n=3 at each time point). The dose volume was 10 ml/kg using appropriate vehicle to solubilize each test compound. At the time of sampling, animals were anesthetized with isoflurane and a systemic blood sample collected by cardiac puncture into vacutainers containing sodium heparin as anti-coagulant. The blood was centrifuged at 3500 rpm for 10 minutes at 4° C. to obtain plasma. Following decapitation, brains were dissected out and transferred to pre-weighed vessels followed by tissue weights determination. Plasma and brains were stored at −80° C. until quantitative bioanalysis by LC-MS/MS. Results are expressed as ng/ml for plasma and ng/g for brain samples.

### Example 6. In Vivo Pharmacology

Effect of Hydroxyurea Vs. Compound P3.1 on HbF Positive and Sickled Red Blood Cells in Berkeley Sickle Cell Transgenic Mice

This study assessed the effects of chronic dosing (30 day) of Compound P3.1 and HU on HbF and cell sickling in a mouse model of sickle cell disease. Berkeley sickle cell transgenic mice (Hbatm1PazHbbtm1Tow Tg(HBA-HBBs)41Paz/J) were divided into groups of 7 to 8 animals and were dosed once daily, by gavage, for 30 days with either vehicle (PEG:water), 30 mg/kg/day of Compound P3.1, or 100 mg/kg/day of HU. This mouse genotype mimics the genetic, hematologic and histopathologic features that are found in humans afflicted with sickle cell anemia, including irreversibly sickled RBCs, anemia and multi-organ pathology. Blood was collected from treated animals on Day 30 for limited routine hematology as well as derivation of the percent of sickled and HbF positive RBCs and total bilirubin. At termination, the spleen was removed and weighed.

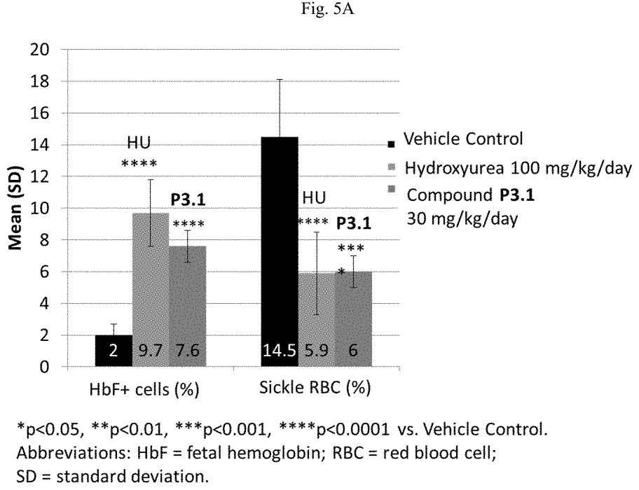

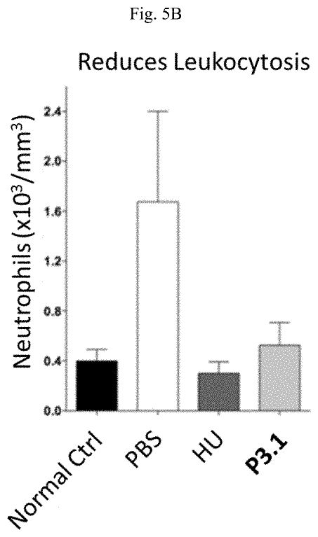

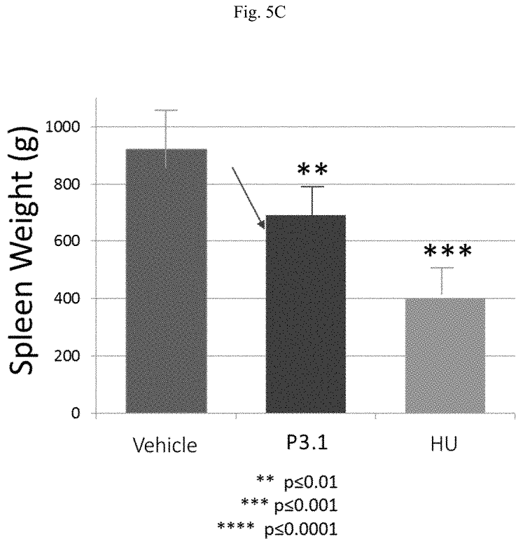

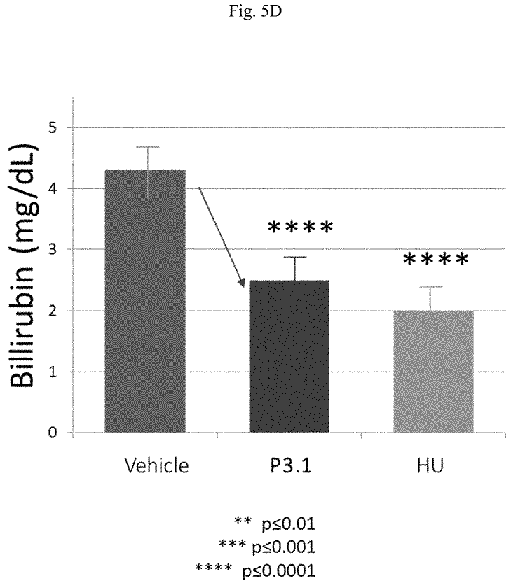

After 30 days of treatment, both Compound P3.1 and HU resulted in statistically significant decreases in the percentage of sickled RBCs and increases in the percentage of HbF positive RBCs relative to controls (FIG. 5A). In addition, both compounds led to statistically significant decreases in total bilirubin, total leucocyte count, and spleen weight. They reduced neutrophil levels and leukotytosis (FIG. 5B). These changes were not associated with any apparent alterations in RBC count, hemoglobin concentration or hematocrit. FIG. 5C shows spleen weights of the mice and demonstrates Compound P3.1 reduces splenomegaly in the mice. FIG. 5D shows bilirubin levels of the mice and demonstrates Compound P3.1 reduces reticulocytosis in the mice.

Administration of 30 mg/kg/day Compound P3.1 for 30 days was well tolerated with no related deaths or abnormal clinical signs. Administration of 100 mg/kg HU was also well tolerated except for 1 death associated with severe anemia.

Effect of Compound P3.1 vs. Hydroxyurea Vs. Compound P3.1 in Combination with Hydroxyurea on Microvascular Stasis and the Percentage of HbF Positive and Sickled Red Blood Cells in HbSS-Townes Mice

The ability of Compound P3.1 to reduce vaso-occulsion was assessed in HbSS-Townes transgenic sickle mice after transient hypoxia and re-oxygenation. This study assessed the effects of repeated (10 days) oral dosing of Compound P3.1 and HU on microvascular stasis and other hematological markers of sickle cell disease, after transient hypoxia and re-oxygenation, in Townes transgenic sickle mice, a mouse model of sickle cell disease (Hba^{tm1(HBA)Tow}Hbb^{tm2(HBG,HBB*)Tow}/Hbb^{tm3(HBG,HBB)Tow}/J) HbSS-Townes mice were divided into groups of 3 mice and were then dosed orally via drinking water with Compound P3.1 at 10 mg/kg/day, Compound P3.1 at 30 mg/kg/day, HU (100 mg/kg/day), or a combination of Compound P3.1 and HU (at doses of 30 and 100 mg/kg/day, respectively) for 10 days. A final group received water containing 0.08% methylcellulose, the vehicle used to prepare the test items, and served as controls. On Day 7 of treatment, the mice were implanted with dorsal skin-fold chambers (DSFC), and on Day 10 of treatment, 20-23 flowing subcutaneous venules in the DSFC window were selected and mapped. After venule selection and mapping, mice were placed in a chamber and exposed to a hypoxic atmosphere (7% O₂/93% N₂) for 1 hour, after which they were returned to room air. All of the selected venules were re-examined after 1 and 4 hours of re-oxygenation in room air and the number of static (no flow) venules was counted and expressed as percent stasis. On completion of these measurements, blood was collected for clinical pathology with a focus on hematological measures associated with sickle cell disease.

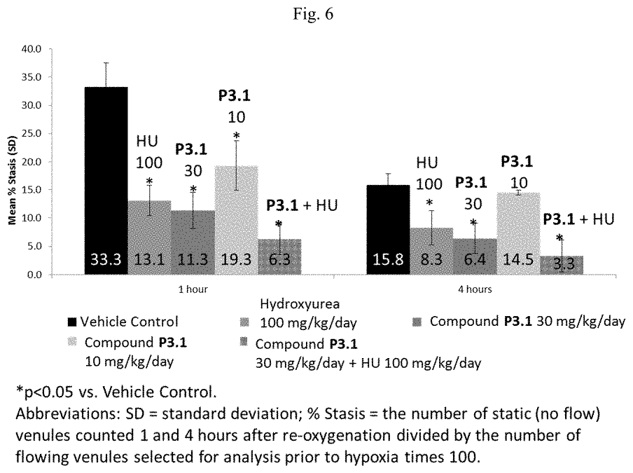

Compared with controls, 30 mg/kg/day Compound P3.1 and 100 mg/kg HU produced statistically significant reductions in stasis at both 1- and 4-hour time points post hypoxia. At 10 mg/kg/day, Compound P3.1 reduced stasis statistically significantly at the 1-hour time point but not at 4 hours. The most effective reduction in microvascular stasis was shown by the combination of Compound P3.1 and HU, where a statistically significant, 5-fold reduction in stasis relative to controls was seen at both time points (FIG. 6).

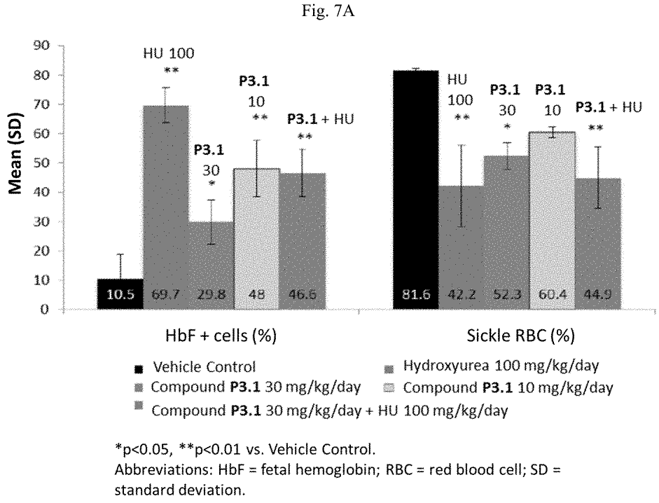

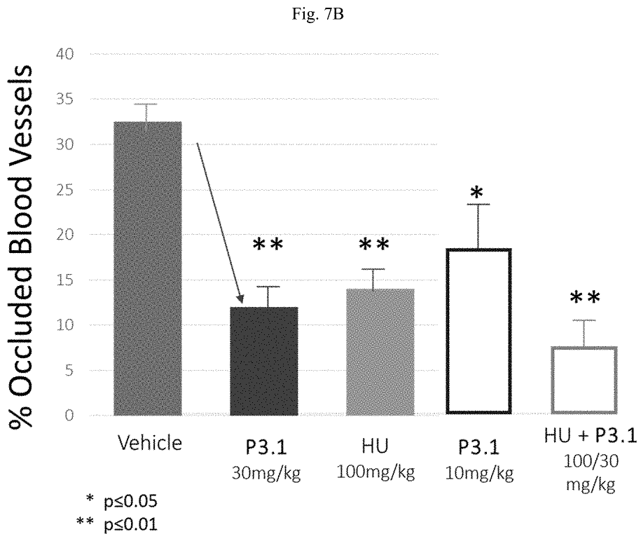

Compound P3.1 given at 30 mg/kg/day produced broadly similar hematological changes to those elicited by the higher, 100 mg/kg/day, dose of HU, most notably the ability to reduce the proportion of sickled RBCs, increase the number of HbF positive red cells and to reduce total WBC numbers. The combination of Compound P3.1 and HU produced similar changes in sickled red cells and HbF cells to those elicited when given alone (FIG. 7A) but led to slightly greater reductions in other hematological measures (total white cell count, hematocrit, heme, and hemoglobin) compared to controls, than when given singly.

As shown in FIG. 7B, vessel occlusion induced by transient hypoxia and re-oxygenation was effectively reduced by 30 mg/kg/day of Compound P3.1 as well as by 100 mg/kg/day of HU, but the greatest reduction in vascular occlusion was elicited by the combination of Compound P3.1 and HU.

Effect of Compound P3.1 vs. AF27873 (Pfizer PDE9 Inhibitor) on Behavior and Biodistribution in C57Bl/6J Mice

This study examined the potential effect of Compound P3.1 and PF-04447943 (also referred to as AF27873), an alternative PDE9 inhibitor originally developed for Alzheimer's disease and currently being developed for SCD by Pfizer, on locomotor activity and memory after 5 days oral administration to C57Bl/6J mice.

The exposure of both compounds was also evaluated in the plasma, brain, and eye. A total of 75 male C57Bl/6J mice, aged 7-8 weeks, were divided into 5 groups each of 15 males, and were dosed by gavage, once daily for 5 days, with either vehicle, Compound P3.1 at 10 or 30 mg/kg/day or AF27873 at 10 or 30 mg/kg/day. During the treatment period all animals were assessed for contextual fear conditioning and a subset of 7 animals per group was evaluated for locomotor activity. On Day 5, plasma, brain, and eye tissue were collected 30 minutes after dosing from 3 animals per treated group to measure concentrations of test item.

Compound P3.1 had no effect on either locomotor activity or memory in this study regardless of dose level administered (10 or 30 mg/kg/day). In contrast, significantly (p<0.05) more conditioned freezing was observed in mice following treatment with 10 mg/kg/day AF27873 compared to vehicle controls; this effect was not observed in mice treated with 30 mg/kg/day AF27873.

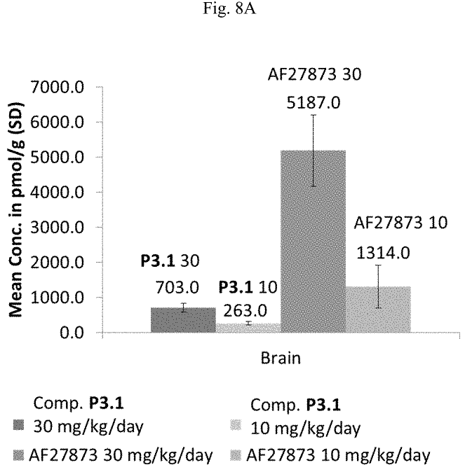

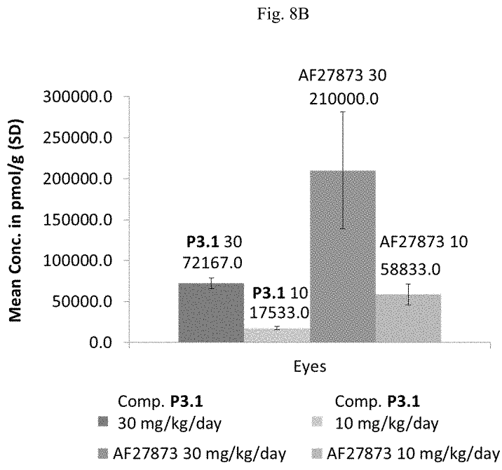

With respect to distribution, Compound P3.1 and AF27873 plasma concentrations were similar to each other, and increased with dose (3837 and 3217 nM, respectively, at 10 mg/kg/day and 9913 and 13100 nM, respectively, at 30 mg/kg/day). In contrast, as shown in FIG. 8A and FIG. 8B, at both the 10- and 30-mg/kg/day dose levels, tissue levels of Compound P3.1 were consistently much lower than those of AF27873 in brain (6- or 7-fold lower) and eye (3-fold lower).

With respect to distribution, Compound P3.1 and AF27873 plasma concentrations were similar to each other, and increased with dose (3837 and 3217 nM, respectively, at 10 mg/kg/day and 9913 and 13100 nM, respectively, at 30 mg/kg/day). In contrast, as shown in FIG. 8A and FIG. 8B, at both the 10- and 30-mg/kg/day dose levels, tissue levels of Compound P3.1 were consistently much lower than those of AF27873 in brain (6- or 7-fold lower) and eye (3-fold lower).

Thus, repeated administration of Compound P3.1, which was associated with very low brain concentrations relative to circulating plasma concentrations (plasma to brain ratio of approximately 14), had no effect on locomotor activity or memory, whereas treatment with AF27873, which resulted in much higher eye and brain concentrations (compared to Compound P3.1) was associated with significantly increased conditioned freezing responses in wild type animals.

In summary, in vitro and in vivo data support the potential efficacy of Compound P3.1 for the treatment of SCD. In vitro, treatment with Compound P3.1 at concentrations of 1, 3, or 10 μM produced dose-dependent and statistically significant increases in cGMP levels at 16 hours and HbF positive cell numbers at 72 hours in the erythroid cell line, K562. Compared to HU, Compound P3.1 was highly potent, with 1 M Compound P3.1 increasing cGMP levels to approximately the same degree as that observed following 100 μM HU, and 3 μM Compound P3.1 increasing HbF-positive cell numbers to approximately the same degree as that observed following 30 or 100 μM HU. Importantly, 10 μM Compound P3.1 also significantly increased HbF levels and the percentage of F cells in CD36+ mature RBCs cultured ex vivo from blood-derived CD34+ cells from 5 SCD subjects. In contrast, treatment with 30 μM HU only increased HbF levels and the percentage of F cells in 3 of 5 parallel CD34+ cell cultures. Further, 2 of the 5 HU-treated CD34+ cell cultures demonstrated <80% viability and were not able to be analyzed.

Repeated or chronic administration of Compound P3.1 also significantly reduced disease-associated pathologies in 2 mouse models of sickle cell disease, the Berkeley and Townes models. In the Berkeley sickle cell transgenic mouse model, which mimics the genetic, hematologic and histopathologic features found in humans afflicted with sickle cell anemia, once daily oral administration of 30 mg/kg Compound P3.1 for 30 days produced statistically significant decreases in the percentage of sickled RBCs and increases in the percentage of HbF positive RBCs relative to the negative control group, both of which were comparable in magnitude to those produced by repeated administration of 100 mg/kg/day HU. Like HU, Compound P3.1 also significantly decreased total bilirubin levels, as well as leucocyte count and spleen weight relative to controls, with no apparent effect on RBC count, hemoglobin concentration, or hematocrit. Oral administration of 30 mg/kg of Compound P3.1 daily for 30 days to Berkeley sickle mice and 10 days to Townes sickle mice was well tolerated with no treatment related deaths or abnormal clinical signs.

Similarly, in the HbSS-Townes sickle cell mouse model, oral administration of 30 mg/kg/day of Compound P3.1 via drinking water for 10 days produced broadly similar hematological changes to those elicited by 100 mg/kg/day HU, most notably the ability to reduce the proportion of sickled RBCs, increase the number of HbF-positive red cells, and reduce total WBC numbers relative to negative controls. Critically, treatment with either Compound P3.1 or HU significantly reduced the degree of microvascular stasis observed following hypoxia and re-oxygenation in these mice. Of note, the greatest reduction in microvascular stasis was observed in mice treated with a combination of 30 mg/kg/day Compound P3.1 and 100 mg/kg/day HU, where a 5-fold reduction in stasis relative to controls was seen.

As previously noted, Compound P3.1 does not efficiently cross the blood brain barrier, reducing the potential for modulation of CNS biology observed with other PDE9 inhibitors. Consistent with this, in C57Bl/6J mice, treatment with 10 or 30 mg/kg/day Compound P3.1 for 5 days had no effect on locomotor activity or classical fear conditioning (an animal model of learning and memory). In contrast, 5 days of treatment with 10 mg/kg/day PF-04447943 (also referred to as AF27873), a PDE9 inhibitor originally developed for the treatment of Alzheimer's disease (Huston et al., Neuropharmacology, 61(4):665-76 (2011); Schwam et al., Curr Alzheimer Res., 11(5):413-21 (2014)) and now being developed for SCD by Pfizer, significantly increased conditioned fear compared to vehicle controls. Moreover, while plasma concentrations of Compound P3.1 and PF-04447943 (AF27873) were similar to each other, tissue levels of Compound P3.1 were consistently much lower than those of AF27873 in both brain (6- to 7-fold lower) and eye (3-fold lower).

### Example 7. Safety Pharmacology

The safety pharmacology assessment of Compound P3.1 included an in vitro hERG assay, neurofunctional and respiratory studies in rats, and a cardiovascular study in beagle dogs.

Superfusion of Compound P3.1 at concentrations up through 10-M did not have an inhibitory effect on hERG-mediated potassium currents.

Single oral doses of 250, 500, and 1000 mg/kg Compound P3.1 in Han Wistar Rats had no effect on clinical observations, home cage observations, handheld observations, or body temperatures. Non-adverse findings considered related to Compound P3.1 included a transient decrease in sensory response (approach response) at the 250-mg/kg dose, and decreases in body weight/weight gain, motor activity (number of rears), and sensory response (tail pinch response) at doses of 500 and 1000 mg/kg. The only adverse finding assessed as related to Compound P3.1 was an increased incidence of no visible-approach response at 0.5 and 24 hours post dosing in animals given >500 mg/kg Compound P3.1.

Single oral administration of Compound P3.1 at doses up to 500 mg/kg in rat had no effect on respiratory functions evaluated; at the highest dose tested of 1000 mg/kg, transient increases in respiratory rate and tidal volume were observed, and these were assessed as related to Compound P3.1. One male rat was found dead at approximately 4.8 hours after receiving 1000 mg/kg Compound P3.1; no abnormal signs were noted. The death was considered test article related. Postmortem and histological examination did not reveal any likely cause of death, and plasma exposure at these levels was in excess of 500,000 ng-h/mL (AUC₀₋₂₄), approximately 48-fold higher than the anticipated efficacious dose, assuming an efficacious dose of 30 mg/kg/day in the mouse.

In a cardiovascular study conducted in 4 dogs, treatments were performed by oral gavage according to a crossover design after allowing a minimum washout period of 48 hours. In conscious dogs, Compound P3.1 at doses of 10 and 25 mg/kg had no effect on arterial blood pressure, heart rate, body temperature, cardiac conduction times, ventricular repolarization duration, QT variability, or ST segment. At the top dose of 75 mg/kg, Compound P3.1 induced tachycardia as well as a slight, progressive and delayed decrease in blood pressure and shortened conduction times.

### Example 8. Pharmacokinetics and Drug Metabolism in Animals

The PK evaluation of Compound P3.1 included Absorption, Distribution, Metabolism, and Excretion (ADME) studies, as well as assessment for CYP enzyme inhibition.

In mice and rats, Compound P3.1 was readily orally absorbed with a time of maximum concentration (Tmax) of 30 minutes to 1 hour and showed high oral bioavailability, with a Flast of 63.4% and 44.6% in rat and mouse, respectively. Compound P3.1 was rapidly cleared with an elimination half-life of ≤3 hours.

In a 14-day repeat-dose toxicology study in rat, Compound P3.1 exposure increased proportionally with dose in males on Days 1 and 14 with the less than proportional increase seen in females on Day 1, becoming dose proportional by Day 14. There was some evidence of increased exposure in females, and no evidence of accumulation over the study.

In a 14-day repeat-dose toxicology study in dog, mean exposures increased with dose in a broadly proportionate manner on Days 1 and 14; the only exception to this was on Day 1, where there was no significant difference between the males given 35 or 75 mg/kg/day. The potential for accumulation could not be evaluated in this study as there was a large variability between individuals.

A comparison of plasma to brain Compound P3.1 concentrations after IV dosing in the rat showed low brain penetration, with plasma concentrations >20 times higher than those in the brain at all time points assessed (out to 4 hours postdose, at which point Compound P3.1 was no longer detectable in the brain).

Based on a comparison to drugs with well-characterized protein binding, Compound P3.1 showed very low plasma protein binding in the 5 species tested, with mean plasma fraction bound (%) values of 23.3% in mouse, 25.2% in rat, 22.9% in dog, 18.6% in monkey, and 31.4% in humans.

Compound P3.1 had no potential for direct inhibition of 7 key CYP enzyme isoforms in human liver microsomes up through the highest concentration tested of 100 μM and no potential for induction of CYP1A2 or CYP2B6 in human hepatocytes.

In a study to evaluate the metabolic stability (intrinsic clearance) of Compound P3.1 in mouse, rat, dog, monkey, and human liver microsomes, Compound P3.1 was highly stable across the species tested, with minimal intrinsic clearance in liver microsomes in either the presence or absence of NADPH.

In a study to evaluate involvement of MDR1 efflux transporter in transport of Compound P3.1 via bi-directional permeability determination across the MDCK-MDR1 cell monolayer, Compound P3.1 was found to be a strong MDR1 (human P-gp) efflux transporter substrate, with an efflux ratio of 180.

In a rat study to assess the toxicokinetics (TK) of Compound P3.1 (250 mg/kg/day) and HU (65 mg/kg/day) when orally administered alone or in combination once daily for 7 days, Compound P3.1 and hydroxyurea were well tolerated when administered alone or in combination, with no deaths or clinical signs during the study and no differences between the groups in mean body weight gain and food intake. Maximal concentration (Cmax) and systemic exposure (area under the concentration-time curve from time 0 to 24 hours postdose [AUC₀₋₂₄]) of HU were approximately 65% lower when given in combination with Compound P3.1 than when given in isolation, but were similar for Compound P3.1 when given in isolation or in combination with HU. In spite of this finding, in the Townes mouse model there was no evidence of reduced activity of HU when combined with Compound P3.1 in reducing the percentage of vessels occluded post hypoxia or in the percentage of red blood cells sickling.

### Pharmacokinetics of Single Oral and IV Compound P3.1 in CD1 Mice and Sprague Dawley Rats

The PK and bioavailability of Compound P3.1 were evaluated in CD1 mice and Sprague Dawley rats following single oral doses at 10 mg/kg or IV doses at 3 mg/kg. Blood samples were taken at 2 minutes (IV only), then 8, 15, 30 minutes, and 1, 2, 4, 8, and 24 hours after dosing and analyzed for key PK parameters. In the rats, brain samples were taken at 24 hours and analyzed for Compound P3.1. To evaluate the penetration of Compound P3.1 across the blood-brain barrier (BBB), 10 additional rats received IV Compound P3.1 3 mg/kg and plasma and brain concentrations of Compound P3.1 were determined from 2 animals at 15 minutes, 30 minutes and 1, 2, and 4 hours after dosing.

Mean PK parameters after IV and oral dosing of Compound P3.1 in mice and rats are shown in Table 3. Compound P3.1 was readily orally absorbed with a Tmax of 30 minutes to 1 hour and showed high oral bioavailability, with an Flast of 63.4% and 44.6% in rat and mouse, respectively. After a 10-mg/kg oral dose, mice were continuously exposed to Compound P3.1 out to 4 hours, and rats were continuously exposed out to 8 hours; by 24 hours plasma concentrations were below the lower limit of quantification (LLOQ) in both species. Similar results were observed in both species following IV administration of Compound P3.1, with plasma concentration below the LLOQ by 24 hours. This clearance reflected the relatively short half-life by both routes. In summary, Compound P3.1 was rapidly cleared with an elimination half-life of ≤3 hours.

TABLE 3
PK Parameters Following Single Dose IV and Oral Administration of Compound P3.1

| | | | | | | | | |
|---|---|---|---|---|---|---|---|---|
| | Cl_obs | | | | | | | |
| Dose & | (mL/min/ | t_{1/2} | tₘₐₓ | C₀ | AUCₗₐₛₜ | MRT | Vss obs | Fₗₐₛₜ |
| Route | kg) | (h) | (h) | (ng/mL) | h*ng/mL | (h) | (L/kg) | (%) |

CD1 Mice

| | | | | | | | | |
|---|---|---|---|---|---|---|---|---|
| 3 mg/kg | 30.7 | 0.63 | n/a | 4430 | 1626 | 0.44 | 0.89 | n/a |
| IV | | | | | | | | |
| 10 mg/kg | n/a | 0.81 | 0.5 | 2747* | 3436 | n/a | 63.4** | 63.4 |
| oral | | | | | | | | |

Sprague Dawley Rats

| | | | | | | | | |
|---|---|---|---|---|---|---|---|---|
| 3 mg/kg | 16.2 | 2.81 | n/a | 4819 | 3112 | 1.44 | 1.65 | n/a |
| IV | | | | | | | | |
| 10 mg/kg | n/a | 3.01 | 1 | 1107* | 4624 | n/a | 44.6 | 44.6 |
| oral | | | | | | | | |

Abbreviations: AUC = area under the concentration time curve from time 0 to the last timepoint; Cl_obs = observed clearance; t_{1/2} = half-life; C₀ = initial or back-extrapolated drug concentration following IV injection; Fₗₐₛₜ = fraction of the dose systemically available; IV = intravenous; MRT = mean residence time; Vss = volume of distribution at steady state.

The comparison of plasma to brain Compound P3.1 concentrations after IV dosing in rat was consistent with low brain penetration, with plasma concentrations being at least 20 times higher than those in the brain (Table 4).

TABLE 4
Compound P3.1 Brain and Plasma Concentrations
Following Single IV Dose of 3 mg/kg

| | Hours | Concentration | | Plasma/ | Mean |
|---|---|---|---|---|---|
| Animal | Post | Compound P3.1 | | Brain | Plasma/Brain |
| # | Dose | Brain | Plasma | Ratio | Ratio |
| 1 | 0.25 | 111 | 2400 | 21.6 | 20.2 |
| 2 | | 112 | 2110 | 18.8 | |
| 3 | 0.5 | 82.5 | 1790 | 21.7 | 25.1 |
| 4 | | 50.7 | 1450 | 28.6 | |
| 5 | 1 | 28.6 | 647 | 22.6 | 23.1 |
| 6 | | 44.1 | 1040 | 23.6 | |
| 7 | 2 | 8.88 | 245 | 27.6 | 33.1 |
| 8 | | 5.34 | 206 | 38.6 | |
| 9 | 4 | N/A | 59.8 | N/A | N/A |
| 10 | | N/A | 107 | N/A | |

Abbreviations
N/A = not available

### Compound P3.1 and Hydroxyurea Drug: Drug Interaction Study in Rat

The TK of high doses of Compound P3.1 (250 mg/kg/day) and HU (65 mg/kg/day) when orally administered alone or in combination once daily for 7 days were evaluated in male rats of the Crl:WI(Han) strain. Animals were observed daily from the start of the dosing and body weights and food intake were recorded at regular intervals. Blood samples were collected from a subset of animals in each group at 6 time points on Day 7 for TK evaluation.

There were no deaths and no clinical signs during the study and mean body weight gain and food intake were similar between groups given Compound P3.1 and HU in isolation or in combination. As show in Table 5, the Cmax and AUC₀₋₂₄ of HU were 63 to 65% lower when given in combination with Compound P3.1 than when given in isolation, while the maximal concentration and systemic exposure of Compound P3.1 were similar when administered either in isolation or combination with HU.

TABLE 5
Maximal Concentrations and Systemic Exposure of
Compound P3.1 and Hydroxyurea

| | | Treatment | | | |
|---|---|---|---|---|---|
| Day | | | | Hydroxyurea and Compound P3.1 | |
| of | Toxicokinetic | Hydroxyurea | Compound P3.1 | Hydroxyurea 65 | Compound P3.1 |
| dosing | parameter | 65 mg/kg/day | 250 mg/kg/day | mg/kg/day | 250 mg/kg/day |
| Day 7 | Cₘₐₓ (ng/mL) | 27 | 16500 | 9.53 | 18800 |
| | AUC₀₋₂₄ | 51.3 | 226000 | 18.6 | 198000 |
| | (ng · h/mL) | | | | |

Abbreviations:
AUC₀₋₂₄ = area under the concentration time curve from time 0 to 24 hours; Cₘₐₓ = maximum concentration.

### Example 9. Toxicology

### Fourteen-Day Repeat-Dose Study in Rats

In a 14-day repeat-dose toxicity study in rats, Compound P3.1 was orally administered (gavage) at doses of 0 (vehicle), 50, 200, and 400 mg/kg/day. At the highest dose of 400 mg/kg/day clinical signs included piloerection, abnormal gait (females only), decreased activity, partially closed eyes, prostration, and slow breathing were observed in both sexes as well as reductions in body weight, weight gain, and food intake, and premature deaths. Postmortem and histological examination did not reveal any likely cause of death and plasma exposure at these levels was in excess of 354,000 ng·h/mL (AUC0-24) approximately >10-fold higher than the anticipated efficacious dose, assuming an efficacious dose of 30 mg/kg/day in the mouse.

A dose level of 200 mg/kg/day in the female rat resulted in intermittent clinical signs and transient, adverse effects on body weight and food intake that resolved before the end of the dosing period; however, microscopic findings were observed in the heart (chronic myocarditis) of a single female. This dose level was well tolerated in the male rat, resulting in non-adverse clinical pathology and microscopic changes (slight hypertrophy in the zona glomerulosa of the adrenals) only. On the basis of these data the no-observed-adverse-effect level (NOAEL) was considered to be 50 mg/kg/day in the female rat and 200 mg/kg/day in the male rat.

As indicated in Table 6, exposure (AUC₀₋₂₄) increased proportionally with dose in males on Days 1 and 14 with the less than proportional increase seen in females on Day 1 becoming dose proportional by Day 14. However, maximal concentrations for both sexes increased subproportionally with dose. There was some evidence of increased exposure in females. There was no clear evidence of accumulation over the study.

TABLE 6
Compound P3.1 Exposures on Days 1 and 14 in Rat

| | 50 mg | | 200 mg | | 400 mg | |
|---|---|---|---|---|---|---|
| Pharmacokinetic | Mean ± SD | | Mean ± SD | | Mean ± SD | |
| Parameters | Day1 | Day 14 | Day 1 | Day 14 | Day 1 | Day 14 |

Males

| | | | | | | |
|---|---|---|---|---|---|---|
| Cₘₐₓ (ng/mL) | 4880 | 7280 | 10900 | 17600 | 38900 | 34900 |
| AUC₀₋₂₄ | 52000 | 67300 | 197000 | 270000 | 354000 | 561000 |
| (ng*hr/mL) | | | | | | |

Females

| | | | | | | |
|---|---|---|---|---|---|---|
| Cₘₐₓ (ng/mL) | 7300 | 8920 | 12600 | 24100 | 22100 | 38900 |
| AUC₀₋₂₄ | 88000 | 97900 | 208000 | 366000 | 371000 | 516000 |
| (ng*hr/mL) | | | | | | |

Abbreviations: AUC₀₋₂₄ = area under the concentration time curve from time 0 to 24 hours; Cₘₐₓ = maximum concentration.

### Fourteen-Day Repeat-Dose Study in Beagle Dogs

In a GLP 14-day repeat-dose toxicity study in dogs, Compound P3.1 was orally administered at doses of 0, 10, 35, or 75 mg/kg/day. Compound P3.1 was associated with emesis, liquid/loose feces, reduced food intake, and losses in body weight in some individuals given 35 or 75 mg/kg/day, with statistically significant weight loss compared to controls in males dosed with 75 mg/kg/day. Increased heart rates were also noted for individuals from all dose groups, although these were not significantly above controls. No deaths were observed at any dose. The no-observed-adverse-effect level (NOAEL) was considered to be 35 mg/kg/day in males and females.

As indicated in Table 7, mean exposures (Cmax and AUC₀₋₂₄) increased with dose in a broadly proportionate manner on Days 1 and 14; the only exception to this was on Day 1, where there was no significant difference between the males given 35 or 75 mg/kg/day.

TABLE 7
Compound P3.1 Exposures on Days 1 and 14 in Dog

| | 10 mg | | 35 mg | | 75 mg | |
|---|---|---|---|---|---|---|
| Pharmacokinetic | Mean ± SD | | Mean ± SD | | Mean ± SD | |
| Parameters | Day 1 | Day 14 | Day 1 | Day 14 | Day 1 | Day 14 |

Males

| | | | | | | |
|---|---|---|---|---|---|---|
| Cₘₐₓ (ng/mL) | 2730 | 3120 | 11200 | 13100 | 11000 | 18300 |
| AUC₀₋₂₄ | 29700 | 24100 | 114000 | 98600 | 137000 | 193000 |
| (ng*hr/mL) | | | | | | |

Females

| | | | | | | |
|---|---|---|---|---|---|---|
| Cₘₐₓ (ng/mL) | 1970 | 3780 | 5180 | 10000 | 13200 | 22100 |
| AUC₀₋₂₄ | 24800 | 25400 | 66700 | 87900 | 152000 | 224000 |
| (ng*hr/mL) | | | | | | |

Abbreviations: AUC₀₋₂₄ = area under the concentration time curve from time 0 to 24 hours; Cₘₐₓ = maximum concentration.

### Rat Fertility Study

In a rat female fertility study, animal groups were given 0, 25, 100, or 200 mg/kg/d oral gavage of Compound P3.1. No treatment-related clinical signs were observed. There was no adverse effect of Compound P3.1 administration on early embryonic development and no effect on pre- or post-implantation loss. There were no macroscopic necropsy findings that indicated an effect of Compound P3.1 administration.

### Genotoxicity

The genotoxicity evaluation of Compound P3.1 consisted of a bacterial reverse mutation assay, a chromosome aberration study, and a in vivo rat micronucleus study. Compound P3.1 was negative in all 3 assays.

In summary, the studies support the safety of Compound P3.1. In nonclinical studies:

Compound P3.1 was generally rapidly absorbed and eliminated in mouse and rat, with an acceptable bioavailability, and a half-life of approximately 3 hours.

Compound P3.1 showed very low plasma protein binding across species, including humans. In a comparison of Compound P3.1 concentrations in plasma vs. brain after IV dosing in the rat, Compound P3.1 demonstrated low brain penetration, with plasma concentrations ≥20 times higher than those in the brain at all time points assessed.

Compound P3.1 was highly stable across species, including humans, with minimal intrinsic clearance in liver microsomes. Moreover, Compound P3.1 showed no inhibitory activity against 7 key CYP enzyme isoforms in human liver microsomes and no induction of CYP1A2 or CYP2B6 in human hepatocytes. However, Compound P3.1 did show potential for induction of CYP3A4.

Compound P3.1 had no significant effects in neurofunctional and respiratory studies in rats at doses up through 250 mg/kg, or in a cardiovascular study in dogs at doses up through 25 mg/kg. Compound P3.1 was also negative in 3 GLP genotoxicity studies, including a bacterial reverse mutation assay, a chromosome aberration assay, and an in vivo rat micronucleus study, and had no inhibitory effect on human ether-à-go-go related gene (hERG)-mediated potassium currents at concentrations up through 10-5M.

In 14-day repeat-dose toxicity studies, the no-observed-adverse-effect-level (NOAEL) in rat was considered to be 200 and 50 mg/kg for males and females, respectively; the NOAEL in the dog was 35 mg/kg in both males and females.

### Example 10. A Phase 1a Single and Multiple Ascending Dose Study of Compound P3.1 in Healthy Adult Volunteers

This study is a Phase 1a, first in human (FIH), randomized, double-blind, placebo-controlled, 2 part study to evaluate the safety, tolerability, and PK effects of orally administered single (Part A) and multiple (Part B) ascending doses of Compound P3.1 in healthy adult subjects. Approximately 5 cohorts of 6 subjects each are planned for Part A, and 3 cohorts of 9 subjects each are planned for Part B. Subjects are randomized 2:1 to Compound P3.1 or placebo. Cohorts (dose levels) are tested sequentially, and initiation of dosing in Part B does not occur until after at least 24 hours of safety and PK data have been evaluated in 3 single-dose cohorts.

For both single- and multiple-dose administration of study drug, the following is assessed: safety and tolerability and the plasma PK profile of Compound P3.1. In addition, the effect of food on the single-dose PK profile of Compound P3.1 is evaluated in Part A.

In Part A, single doses of Compound P3.1 or placebo are evaluated at 0.3 mg/kg per day (mg/kg/d) (Cohort 1), 1 mg/kg/d (Cohort 2), 3 mg/kg/d (Cohort 3), 10 mg/kg/d (Cohort 4), and 30 mg/kg/d (Cohort 5). A sixth cohort may be enrolled to test an intermediate dose level. Subjects are admitted to the clinical study unit on the day prior to dosing and receive a single oral dose of study drug on Day 1 following an overnight fast; subjects remain confined to the study unit through completion of the last assessment on Day 2 and for at least 24 hours after dose administration.

Safety follow-up is evaluated on Day 5. Subjects enrolled in the 3-mg/kg dose cohort return to the clinic at least 7 days after study drug administration in the fasted state and receive a single dose of study drug (according to their original randomization) approximately 1 hour following a standard high-fat breakfast.

In Part B, multiple doses of Compound P3.1 or placebo are evaluated at 1 mg/kg (Cohort 1), 3 mg/kg (Cohort 2), and 10 mg/kg (Cohort 3). Subjects are admitted to the clinical study unit on the day prior to dosing and receive study drug orally once daily on Days 1 through 7 approximately 1 hour following a meal; subjects remain confined to the study unit through completion of the last assessment on Day 8 and for at least 24 hours after dose administration. Safety follow-up is evaluated on Day 12.

### Example 11. A Phase 1b, Randomized, Double-Blind, Placebo-Controlled Study of Compound P3.1 in Adult Subjects with Sickle Cell Disease

This study is a Phase 1b, randomized, double-blind, placebo-controlled study to evaluate the safety, tolerability, PK, PD, and clinical outcomes of Compound P3.1 in adult subjects with a confirmed diagnosis of SCD. A total of 36 subjects are enrolled with the goal of having 32 subjects complete the study. Eligible subjects are randomized 3:1 to receive oral doses Compound P3.1 or placebo QD for up to 24 weeks at 10 mg/kg (or, if lower, at the maximum tolerated dose (MTD) as determined in previous study. Subjects remain at the clinical site for 24 hours following the first dose of study drug; subjects return to the site on an outpatient basis for the remaining study visits. No subject is dosed beyond 12 weeks unless it is determined that it is safe and appropriate to continue dosing based on both the available nonclinical (6-month data from rat and dog toxicity studies and a rat fertility study) and clinical (all available safety data once the first subject has received 8 weeks of study drug) data.

Study measures include: the safety and tolerability, plasma PK profile, PD effects, and clinical outcome effects of Compound P3.1 in adult subjects with SCD.

Pharmacodynamic (PD) effects are assessed by changes from baseline in total Hb, HbF, cGMP, reticulocyte counts, indices of red cell hemolysis, and neutrophil counts. Effects on clinical outcomes are assessed by changes from baseline in pain; the physical, social, and emotional impact of SCD; the use of pain medications; and the occurrence of SCD-related events requiring medical or health care professional attention and/or hospitalization, including VOCs and the number and frequency of transfusions.

### Example 12. A Phase 2a, Randomized, Double-Blind, Placebo-Controlled Study of Compound P3.1 in Children and Adolescent Subjects with Sickle Cell Disease

This study is a Phase 2a randomized, double-blind, placebo-controlled study to evaluate the safety, tolerability, PK, PD, and clinical outcomes of Compound P3.1 in children and adolescent subjects (≥8 and ≤18 years of age) with a confirmed diagnosis of SCD. A total of 60 subjects are enrolled with the goal of having 54 subjects complete the study. Eligible subjects are randomized 2:1 to receive Compound P3.1 or placebo for 24 weeks in 1 of 2 sequentially enrolled dosing cohorts. Subjects in Cohort 1 receive Compound P3.1 or placebo once daily at 3 mg/kg (or, if lower, at one-third the MTD as determined in the Phase 1a study); subjects in Cohort 2 receive Compound P3.1 or placebo once daily at 10 mg/kg (or, if lower, at the MTD in previous study). Subjects remain at the clinical site for 24 hours following the first dose of study drug and return to the site on an outpatient basis for the remaining study visits. Dosing in this study is not initiated until data from a juvenile rat toxicity study are available to support dosing in children and adolescents. Dosing in Cohort 2 is not initiated until the first 9 subjects in Cohort 1 have completed at least 12 weeks of treatment and all available safety data from all subjects have been evaluated by the SRC.

Study measures include: the safety and tolerability, plasma PK profile, PD effects, and clinical outcome effects of Compound P3.1 in children and adolescents with SCD. PD effects are assessed by changes from baseline in total Hb, HbF, cGMP, reticulocyte counts, indices of red cell hemolysis, and neutrophil counts. Effects on clinical outcomes are assessed by changes from baseline in pain; the physical, social, and emotional impact of SCD; the use of pain medications; and the occurrence of SCD-related events requiring medical or health care professional attention and/or hospitalization, including VOCs and the number and frequency of transfusions.

### Example 13. TNFa Activated Human Cells: Compound P3.1 Reduces Neutrophil Adhesion in Micro-Channel Assay

### Objective

This in vitro study has the aim of analyzing the effects of Compound P3.1 on the properties of circulating polymorphonuclear neutrophils (PMN) and of human endothelial cells.

In this study, the effects of Compound P3.1 on PMN adhesion to TNF-α activated human endothelial cell monolayers under flow conditions are investigated. It has been validated in vitro dynamic assay mimics neutrophil recruitment to endothelial cell monolayers under inflammatory conditions (TNF-α activation). In this assay, both control and sickle PMNs adhere to endothelial cells but to different degrees, reflecting in vivo conditions. The human dermal microvascular endothelial cell line HMEC-1 is used in this approach. The potential inhibitory effect of Compound P3.1 is tested in parallel and compared with the effect of HU and other PDE9 inhibitors.

In a first step, the effects of 1 uM Compound P3.1 and 10 uM HU were tested by incubating blood samples from healthy volunteers (n=3-6) (i.e., donors or donor cells) with these molecules.

### Methods

Adhesion under flow conditions (fresh blood), sickle cell anemia (SCA) PMNs are highly adhesive to endothelial cells. This increased adhesion is believed to initiate or to contribute to VOC.

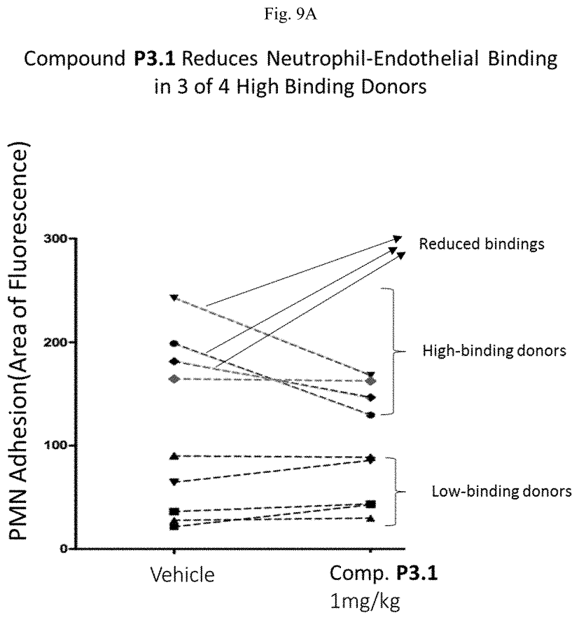

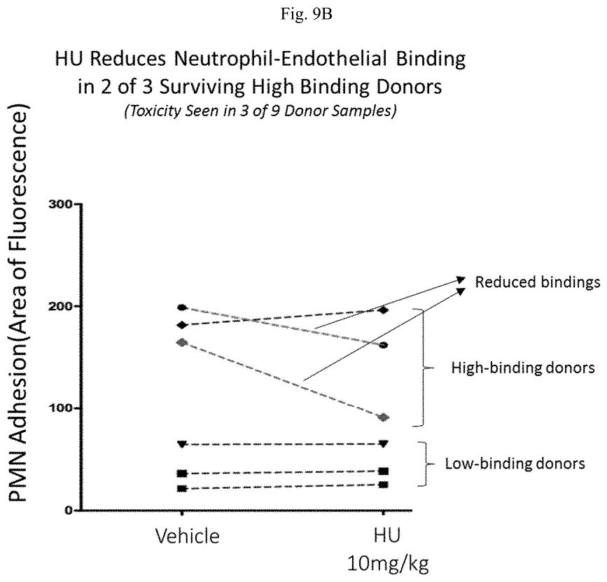

Adhesion of PMNs from healthy volunteers was assessed under flow conditions, mimicking blood flow, in micro channels (Venaflux, Cellix, Ireland) coated with endothelial cell monolayers cultivated under inflammatory conditions. The adhesion assay was performed with whole fresh blood, previously incubated or not with Compound P3.1, to be closer to the physiological condition of circulation in a person, and to study the interaction between the different blood cells with PMNs. Results are shown in FIG. 9A and FIG. 9B.

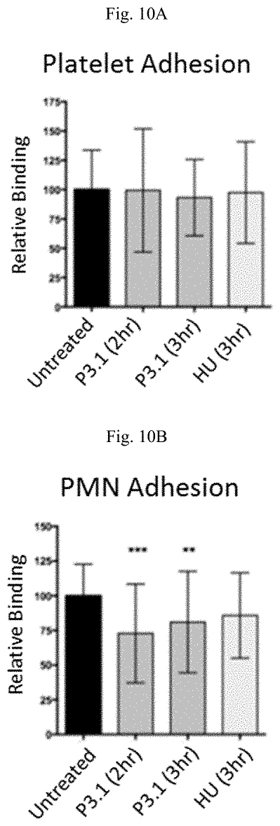

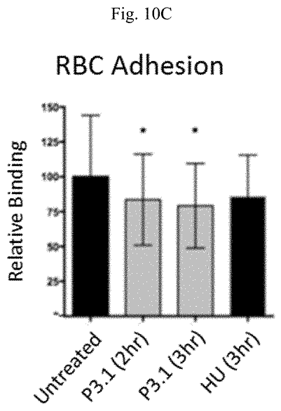

In another assay, using the same method, the reduction of PMN and RBC bindings to TNF-α activated endothelium in a micro channel well was confirmed. Platelet, PMN, and RBC in blood samples from 5 healthy normal volunteers (donors) were labelled with fluorescent dyes. The blood samples were incubated with Compound P3.1 for 2 hours or 3 hours, or HU for 3 hours, prior to running them on micro channels that were previously coated with endothelial cells activated with TNF-α. The % of bound cells was quantified at 30 min. Results are shown in FIG. 10A, FIG. 10B, and FIG. 10C.

### CONCLUSIONS

As shown in FIG. 9A and FIG. 9B, untreated donor cells (Vehicles in FIG. 9A and FIG. 9B) either demonstrate high levels of binding to TNF-α activated endothelial cell coated microchannels (>150 fluorescent units) (High-binding donors in FIG. 9A and FIG. 9B) or low levels of binding to activated endothelial cells (<100 fluorescent units) (Low-binding donors in FIG. 9A and FIG. 9B). Compound P3.1 treatment has no effect on the low-binding donors. Compound P3.1 treated neutrophils reduced the adhesion of 3 of 4 high-binding donors and had no effect on one of the high-binding donors. HU treatment reduced the binding of 2 of 3 of the high binding donors and had no effect on one of the high-binding donors. Of note, the HU treatment demonstrated toxicity, and 3 of 9 of the donor samples did not survive HU treatment.

PMNs bind to the endothelial cells first. Then red blood cells (RBC) bind to PMNs and then platelets to RBCs. As shown in FIGS. 10B and 10C, Compound P3.1 reduced PMN and RBC adhesions to endothelial cells. Interestingly, treatments with Compound P3.1 or HU did not impact platelet binding (FIG. 10A), suggesting that the treatments had no effect on P-selectin.

1.-31. (canceled)

32. A pharmaceutical composition comprising
a PDE9 inhibitor having the structure of Formula (I): 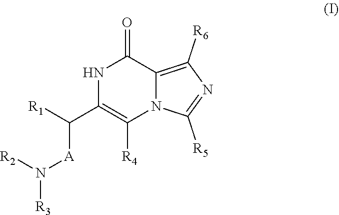
wherein R2 is cyclized with either R1 or R3,
wherein R1, R2 and R3 are
R1, when cyclized with R2, is 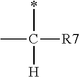
wherein R7 is selected from the group consisting of H, —CH₃, —C₂H₅, and —C₃H₇,
wherein * denotes the cyclization point, and
R1, when not cyclized, is selected from the group consisting of
H and 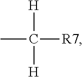
wherein R7 is selected from the group consisting of H, —CH₃, —C₂H₅, and —C₃H₇,
R2 is a compound selected from the group consisting of 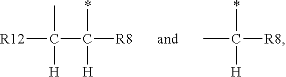
wherein R8 and R12 independently are selected from the group consisting of H, —CH₃, —C₂H₅, and —C₃H₇
wherein * denotes the cyclization point, and
R3, when cyclized with R2, is 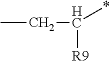
wherein * denotes the cyclization point, and
wherein R9 is selected from the group consisting of H, C₁-C₆ alkyl, substituted C₁-C₆ alkyl, branched C₃-C₆ alkyl, C₃-C₆ cycloalkyl, substituted C₃-C₆ cycloalkyl, C₆-C₁₀ aryl, substituted C₆-C₁₀ aryl, C₃-C₉ heteroaryl, substituted C₃-C₉ heteroaryl, C₁-C₆ alkoxy, substituted C₁-C₆ alkoxy, branched C₃-C₆ alkoxy, C₃-C₆ cycloalkoxy, substituted C₃-C₆ cycloalkoxy, C₆-C₁₀ aryloxy, substituted C₆-C₁₀ aryloxy, C₃-C₉ heteroaryloxy, substituted C₃-C₉ heteroaryloxy; and
R3, when not cyclized, is 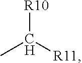
wherein
R10 is selected from the group consisting of H, —CH₃, and —C₂H₅; and
R11 is selected from the group consisting of C₆-C₁₀ aryl, substituted C₆-C₁₀ aryl, C₃-C₉ heteroaryl, substituted C₃-C₉ heteroaryl;
R4 is selected from the group consisting of hydrogen, —CH₃, —C₂H₅, —C₃H₇, —CF₃, —CN, F and Cl;
R5 is selected from the group consisting of C₆-C₁₀ aryl, substituted C₆-C₁₀ aryl, C₃-C₉ heteroaryl, substituted C₃-C₉ heteroaryl, C₃-C₆ heterocyclyl, substituted C₃-C₆ heterocyclyl, C₃-C₆ cycloalkyl, and substituted C₃-C₆ cycloalkyl;
R6 is selected from the group consisting of hydrogen, F, Cl, CN, —CH₃, —C₂H₅, —C₃H₇, and —CF₃;
A is absent or —CH₂; and
hydroxyurea (HU).

33. The pharmaceutical composition of claim 32, wherein the PDE9 inhibitor of Formula (I) is selected from the group consisting of: 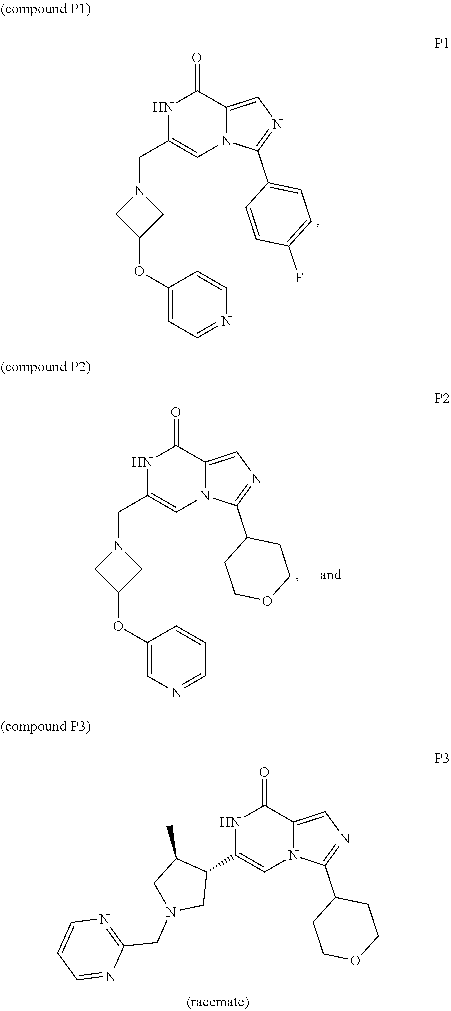 [SMILES: O=c1[nH]c(CN2CC(Oc3ccncc3)C2)cn2c(-c3ccc(F)cc3)ncc12 ; O=c1[nH]c(CN2CC(Oc3cccnc3)C2)cn2c(C3CCOCC3)ncc12 ; C[C@@H]1CN(Cc2ncccn2)C[C@H]1c1cn2c(C3CCOCC3)ncc2c(=O)[nH]1]
in racemic form and in enantiomerically enriched or pure form.

34. The pharmaceutical composition of claim 32, wherein the ratio between the PDE9 inhibitor and HU is between 1:500 to 500:1, between 1:100 to 100:1, between 1:50 to 50:1, between 1:20 to 20:1, between 1:5 to 5:1, or 1:1.

35. The pharmaceutical composition of claim 32, wherein the PDE9 inhibitor of Formula (I) is administered to a patient at between about 0.3 mg/kg to about 500 mg/kg.

36. The pharmaceutical composition of claim 32, wherein the PDE9 inhibitor of Formula (I) is administered to a patient at about 0.3 mg/kg, about 1 mg/kg, about 3 mg/kg, about 10 mg/kg, about 30 mg/kg, about 50 mg/kg, about 100 mg/kg, about 150 mg/kg, about 200 mg/kg, or about 250 mg/kg.

37. A method of treating sickle cell disease (SCD) in a patient in need thereof, the method comprising administering to the patient the pharmaceutical composition comprising a PDE9 inhibitor having the structure of Formula (I): 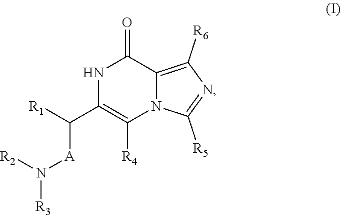
wherein R2 is cyclized with either R1 or R3,
wherein R1, R2 and R3 are
R1, when cyclized with R2, is 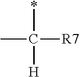
wherein R7 is selected from the group consisting of H, —CH₃, —C₂H₅, and —C₃H₇,
wherein * denotes the cyclization point, and
R1, when not cyclized, is selected from the group consisting of
H and 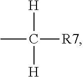
wherein R7 is selected from the group consisting of H, —CH₃, —C₂H₅, and —C₃H₇,
R2 is a compound selected from the group consisting of 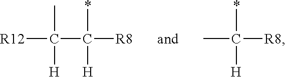
wherein R8 and R12 independently are selected from the group consisting of H, —CH₃, —C₂H₅, and —C₃H₇
wherein * denotes the cyclization point, and
R3, when cyclized with R2, is 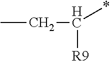
wherein * denotes the cyclization point, and
wherein R9 is selected from the group consisting of H, C₁-C₆ alkyl, substituted C₁-C₆ alkyl, branched C₃-C₆ alkyl, C₃-C₆ cycloalkyl, substituted C₃-C₆ cycloalkyl, C₆-C₁₀ aryl, substituted C₆-C₁₀ aryl, C₃-C₉ heteroaryl, substituted C₃-C₉ heteroaryl, C₁-C₆ alkoxy, substituted C₁-C₆ alkoxy, branched C₃-C₆ alkoxy, C₃-C₆ cycloalkoxy, substituted C₃-C₆ cycloalkoxy, C₆-C₁₀ aryloxy, substituted C₆-C₁₀ aryloxy, C₃-C₉ heteroaryloxy, substituted C₃-C₉ heteroaryloxy; and
R3, when not cyclized, is 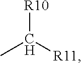
wherein
R10 is selected from the group consisting of H, —CH₃, and —C₂H₅; and
R11 is selected from the group consisting of C₆-C₁₀ aryl, substituted C₆-C₁₀ aryl, C₃-C₉ heteroaryl, substituted C₃-C₉ heteroaryl;
R4 is selected from the group consisting of hydrogen, —CH₃, —C₂H₅, —C₃H₇, —CF₃, —CN, F and Cl;
R5 is selected from the group consisting of C₆-C₁₀ aryl, substituted C₆-C₁₀ aryl, C₃-C₉ heteroaryl, substituted C₃-C₉ heteroaryl, C₃-C₆ heterocyclyl, substituted C₃-C₆ heterocyclyl, C₃-C₆ cycloalkyl, and substituted C₃-C₆ cycloalkyl;
R6 is selected from the group consisting of hydrogen, F, Cl, CN, —CH₃, —C₂H₅, —C₃H₇, and —CF₃;
A is absent or —CH₂; and
hydroxyurea (HU).

38. The method of claim 37, wherein the pharmaceutical composition is administered orally.

39. The method of claim 37, wherein the pharmaceutical composition is administered daily.

40. The method of claim 37, wherein the pharmaceutical composition is administered concurrently or sequentially.

41. The method of claim 37, wherein the pharmaceutical composition is administered for between 1 to 7 days.

42. The method of claim 37, wherein the pharmaceutical composition is administered for at least 7 days.

43. A pharmaceutical composition comprising a PDE9 inhibitor, hydroxyurea (HU), and one or more pharmaceutically acceptable carriers, diluents or excipients, wherein the PDE9 inhibitor is 6-[(3S,4S)-4-methyl-1-(pyrimidin-2-ylmethyl)pyrrolidin-3-yl]-3-tetrahydropyran-4-yl-7H-imidazo[1,5-a]pyrazin-8-one (compound P3.1).

44. The pharmaceutical composition of claim 43, wherein the ratio between the PDE9 inhibitor and HU is between 1:500 to 500:1, between 1:100 to 100:1, between 1:50 to 50:1, between 1:20 to 20:1, between 1:5 to 5:1, or 1:1.

45. The pharmaceutical composition of claim 43, wherein the PDE9 inhibitor of Formula (I) is administered to a patient at between about 0.3 mg/kg to about 500 mg/kg.

46. The pharmaceutical composition of claim 43, wherein the PDE9 inhibitor of Formula (I) is administered to a patient at about 0.3 mg/kg, about 1 mg/kg, about 3 mg/kg, about 10 mg/kg, about 30 mg/kg, about 50 mg/kg, about 100 mg/kg, about 150 mg/kg, about 200 mg/kg, or about 250 mg/kg.
